(19) United States
(12) Patent Application Publication
(10) Pub. No.: US 2017/0145433 A1
(43) Pub. Date: May 25, 2017

(54) LOWERING SATURATED FATTY ACID CONTENT OF PLANT SEEDS

(71) Applicant: Dow AgroSciences LLC, Indianapolis, IN (US)

(72) Inventors: Ann Owens MERLO, Carmel, IN (US); Daniel J. Gachotte, Indianapolis, IN (US); Mark A. Thompson, Zionsville, IN (US); Terence A. Walsh, Indianapolis, IN (US)

(21) Appl. No.: 15/380,891

(22) Filed: Dec. 15, 2016

Related U.S. Application Data
(63) Continuation-in-part of application No. 13/168,742, filed on Jun. 24, 2011.
Application No. 13/168,742 is a continuation-in-part of application No. 11/576,750, filed on Mar. 14, 2008.
Application No. 11/576,750 is a continuation of application No. 60/617,532, filed on Oct. 8, 2004.
(60) Provisional application No. 61/358,314, filed on Jun. 24, 2010.

(51) Int. Cl. C12N 15/82; C12N 9/02

(52) U.S. Cl. CPC C12N 15/8247; C12N 9/0083; C12Y 114/99

(57) ABSTRACT

Compositions and methods include genetically encoding and expressing a novel delta-9 desaturase in plant cells. In some embodiments, methods of expressing nucleic acids in a plant cell to take advantage of the delta-9 desaturase enzyme's activity, such that the percent composition of saturated fatty acids in plant seeds is decreased and there is a concomitant increase in ω-7 fatty acids. In other embodiments, amino acid sequences have delta-9 desaturase activity. Methods can involve expression of delta-9 desaturase in plant cells, plant materials, and whole plants for the purpose of increasing the amount of unusual fatty acids in whole plants, plant seeds, and plant materials, for example, seeds.

### CROSS-REFERENCE TO RELATED APPLICATIONS

This application is a continuation-in-part of co-pending application U.S. Ser. No. 13/168,742 filed Jun. 24, 2011, which is a continuation-in-part of co-pending application U.S. Ser. No. 11/576,750 filed Mar. 14, 2008, which is a national phase entry of PCT International Patent Application No. PCT/US05/36052, filed Oct. 7, 2005, designating the United States of America, and published, in English, as PCT International Publication No. WO 2006/042049 A2 on Apr. 20, 2006. PCT International Patent Application No. PCT/US05/36052 is a continuation of U.S. Provisional Patent Application No. 60/617,532, filed Oct. 8, 2004. U.S. application Ser. No. 13/168,742 filed Jun. 24, 2011 also claims priority to U.S. Provisional Patent Application No. 61/358,314, filed Jun. 24, 2010. The contents of the entirety of each of the foregoing are hereby incorporated herein by this reference.

### FIELD OF THE INVENTION

Some embodiments generally relate to certain delta-9 desaturase enzymes, nucleic acids encoding these enzymes, and methods of expressing the same in a plant cell. Some embodiments relate to utilizing the activity of certain delta-9 desaturase enzymes to decrease the percent composition of saturated fatty acids in plant materials (e.g., seed) and increasing the percent composition of ω-7 fatty acids. Also disclosed herein are plants and plant materials produced by methods in particular embodiments.

### BACKGROUND

Vegetable-derived oils have gradually replaced animal-derived oils and fats as the major source of dietary fat intake. However, saturated fat intake in most industrialized nations has remained at about 15% to 20% of total caloric consumption. In efforts to promote healthier lifestyles, the United States Department of Agriculture (USDA) has recently recommended that saturated fats make up less than 10% of daily caloric intake. To facilitate consumer awareness, current labeling guidelines issued by the USDA now require total saturated fatty acid levels be less than 1.0 g per 14 g serving to receive the “low-sat” label and less than 0.5 g per 14 g serving to receive the “no-sat” label. This means that the saturated fatty acid content of plant oils needs to be less than 7% and 3.5% to receive the “low-sat” or “no-sat” label, respectively. Since issuance of these guidelines, there has been a surge in consumer demand for “low-sat” and “no-sat” oils. To date, this demand has been met principally with canola oil, and to a much lesser degree with sunflower and safflower oils.

While unsaturated fats (monounsaturated and polyunsaturated) are beneficial (especially when consumed in moderation), saturated and trans fats are not. Saturated fat and trans fat raise undesirable LDL cholesterol levels in the blood. Dietary cholesterol also raises LDL cholesterol and may contribute to heart disease even without raising LDL. Therefore, it is advisable to choose foods low in saturated fat, trans fat, and cholesterol as part of a healthful diet.

The characteristics of oils, whether of plant or animal origin, are determined predominately by the number of carbon and hydrogen atoms in the oil molecule, as well as the number and position of double bonds comprised in the fatty acid chain. Most oils derived from plants are composed of varying amounts of palmitic (16:0), stearic (18:0), oleic (18:1), linoleic (18:2) and linolenic (18:3) fatty acids. Conventionally, palmitic and stearic acids are designated as “saturated,” because their carbon chains are saturated with hydrogen atoms, and hence have no double bonds; they contain the maximal number of hydrogen atoms possible. However, oleic, linoleic, and linolenic acids are 18-carbon fatty acid chains having one, two, and three double bonds, respectively, therein. Oleic acid is typically considered a monounsaturated fatty acid, whereas linoleic and linolenic are considered to be polyunsaturated fatty acids. The U.S.D.A. definition of “no sat” oil products as those having less than 3.5% fatty acid content is calculated as the combined saturated fatty acid content by weight (as compared to the total amount of fatty acids).

Canola oil has the lowest level of saturated fatty acids of all vegetable oils. “Canola” refers to rapeseed (Brassica) which has an erucic acid (C22:1) content of at most 2% by weight, based on the total fatty acid content of a seed (preferably at most 0.5% by weight, and most preferably essentially 0% by weight), and which produces, after crushing, an air-dried meal containing less than 30 μmol/g of defatted (oil-free) meal. These types of rapeseed are distinguished by their edibility in comparison to more traditional varieties of the species.

It is postulated that, in oilseeds, fatty acid synthesis occurs primarily in the plastid. The major product of fatty acid synthesis is palmitate (16:0), which appears to be efficiently elongated to stearate (18:0). While still in the plastid, the saturated fatty acids may then be desaturated by an enzyme known as acyl-ACP delta-9 desaturase, to introduce one or more carbon-carbon double bonds. Specifically, stearate may be rapidly desaturated by a plastidial delta-9 desaturase enzyme to yield oleate (18:1). In fact, palmitate may also be desaturated to palmitoleate (16:1) by the plastidial delta-9 desaturase, but this fatty acid appears in only trace quantities (0-0.2%) in most vegetable oils. Thus, the major products of fatty acid synthesis in the plastid are palmitate, stearate, and oleate. In most oils, oleate is the major fatty acid synthesized, as the saturated fatty acids are present in much lower proportions.

Newly-synthesized fatty acids are exported from the plastid to the cytoplasm. Subsequent desaturation of plant fatty acids in the cytoplasm appears to be limited to oleate, which may be desaturated to linoleate (18:2) and linolenate (18:3) by microsomal desaturases acting on oleoyl or lineoleoyl substrates esterified to phosphatidyl choline (PC). In addition, depending on the plant, oleate may be further modified by elongation (to 20:1, 22:1, and/or 24:1), or by the addition of functional groups. These fatty acids, along with the saturated fatty acids, palmitate and stearate, are then assembled into triglycerides in endoreticular membranes.

The plant acyl-ACP delta-9 desaturase enzyme is soluble. It is located in the plastid stroma, and uses newly-synthesized fatty acids esterified to ACP, predominantly stearyl-ACP, as substrates. This is in contrast to the other delta-9 desaturase enzymes, which are located in the endoplasmic reticular membrane (ER, or microsomal), use fatty acids esterified to Co-A as substrates, and desaturate both the saturated fatty acids, palmitate and stearate. U.S. Pat. Nos. 5,723,595 and 6,706,950 relate to a plant desaturase.

The yeast delta-9 desaturase gene has been isolated from Saccharomyces cerevisiae, cloned, and sequenced. Stukey et al. (1989) J. Biol. Chem. 264:16537-44; Stukey et al. (1990) J. Biol. Chem. 265:20144-9. This yeast gene has been introduced into tobacco leaf tissue (Polashcok et al. (1991) FASEB J. 5:A1157; Polashok et al. (1992) Plant Physiol. 100:894-901), and was apparently expressed in this tissue. Further, this yeast gene was expressed in tomato. See Wang et al. (1996) J. Agric. Food Chem. 44:3399-402; and Wang et al. (2001) Phytochemistry 58:227-32. While some increases in certain unsaturated fatty acids, and some decreases in certain saturated fatty acids, were reported for both tobacco and tomato using this yeast delta-9 desaturase gene, tobacco and tomato are clearly not oil crops. This yeast gene was also introduced into Brassica napus. U.S. Pat. No. 5,777,201.

A different fungal acyl-CoA delta-9 desaturase from Aspergillus nidulans has been introduced into canola, thereby achieving reduced saturated fatty acid levels in seed oil. U.S. Patent Application Publication US 2008/0260933 A1. The A. nidulans acyl-CoA delta-9 desaturase provided greater depletion of stearate (61-90%) than the more abundant palmitate fatty acids (36-49%) in the seed oil.

### BRIEF SUMMARY OF THE INVENTION

Disclosed herein are novel fungal delta-9 desaturase enzymes; nucleic acids comprising at least one nucleotide sequence encoding such a desaturase; and plants, plant materials (e.g., seed), plant parts, and plant commodity products comprising either of the foregoing. Aspects of some embodiments are exemplified by fungal delta-9 desaturase enzymes isolated from Magnaporthe grisea, Leptosphaeria nodorum, and Helicoverpa zea. Some examples include native and synthetic delta-9 desaturases that have a substrate preference for palmitic acid or stearic acid.

Some embodiments comprise an isolated nucleic acid molecule encoding a delta-9 desaturase enzyme comprising an amino acid sequence being at least 80% identical to a sequence selected from the group consisting of SEQ ID NO:12, SEQ ID NO:13, SEQ ID NO:14, SEQ ID NO:50, SEQ ID NO:51, SEQ ID NO:52, SEQ ID NO:72, and SEQ ID NO:73. In particular examples, the nucleic acid molecule comprises a sequence being at least 60% identical to a sequence selected from the group consisting of SEQ ID NO:3, SEQ ID NO:4, SEQ ID NO:5, SEQ ID NO:8, SEQ ID NO:9, SEQ ID NO:10, SEQ ID NO:11, SEQ ID NO:15, SEQ ID NO:16, SEQ ID NO:17, SEQ ID NO:44, SEQ ID NO:45, SEQ ID NO:48, and SEQ ID NO:49. These and further embodiments may include an isolated delta-9 desaturase polypeptide comprising an amino acid sequence being at least 80% identical to a sequence selected from the group consisting of SEQ ID NO:12, SEQ ID NO:13, SEQ ID NO:14, SEQ ID NO:50, SEQ ID NO:51, SEQ ID NO:52, SEQ ID NO:72, and SEQ ID NO:73.

Also disclosed are methods of expressing at least one of the aforementioned nucleic acids and/or polypeptides in a plant cell. Particular embodiments take advantage of a delta-9 desaturase enzyme's activity, such that the percent composition of saturated fatty acids may be decreased in a plant, plant material (e.g., seed), and/or plant part comprising the plant cell, and/or a plant commodity product produced from any of the foregoing. In certain embodiments, ω-7 fatty acids may concomitantly be increased in the plant, plant material, plant part, and/or plant commodity product.

Some embodiments include a method for decreasing the amount of saturated fatty acids in a plant, plant material, plant part, and/or plant commodity product, the method comprising transforming a plant cell with a nucleic acid molecule encoding a delta-9 desaturase polypeptide of the invention, such that the amount of saturated fatty acids in the cell is decreased. Some embodiments include a method for creating a genetically engineered plant that comprises decreased amounts of saturated fatty acids in the plant compared to a wild-type plant of the same species. Such a method may comprise transforming a plant material (or plant cell) with a nucleic acid molecule encoding a delta-9 desaturase polypeptide of the invention, and culturing the transformed plant material (or plant cell) to obtain a plant. In particular examples, a plant cell and/or plant material from an Arabidopsis sp. may be transformed with a nucleic acid molecule encoding a delta-9 desaturase polypeptide of the invention.

The foregoing and other features will become more apparent from the following detailed description of several embodiments, which proceeds with reference to the accompanying figures.

### BRIEF DESCRIPTION OF THE FIGURES

FIG. 1 includes a schematic phylogenetic analysis of various fungal desaturase protein sequences. The complete protein sequences of the depicted desaturases were aligned using ClustalX and displayed using MEGA.

FIGS. 2(a-d) include an alignment of fungal delta-9 desaturase gene sequences. Capital font represents conserved nucleotides in this alignment. Shaded font represents identical nucleotides in this alignment.

FIGS. 3(a-b) include an alignment of fungal delta-9 desaturase polypeptides.

FIGS. 4(a-b)-18 include plasmid maps of exemplary plasmids comprising fungal delta-9 desaturase polypeptide-encoding nucleotide sequences that may be useful in some embodiments. FIGS. 4a and 4b specifically include plasmid maps of exemplary plasmids comprising LnD9DS-2-encoding (FIG. 4a; pDAB110110) and HzD9DS-encoding (FIG. 4b; pDAB110112) nucleotide sequences that further comprise the PvPhas 5′ UTR and PvPhas 3′ UTR.

FIG. 19 includes data showing the total saturated fatty acid content (% FAMEs) of exemplary T₂ Arabidopsis seed from plants transformed with certain exemplary fungal delta-9 desaturase gene sequences.

FIG. 20 includes data showing the palmitic acid (C16:0) content (% FAMEs) of exemplary T₂ Arabidopsis seed from plants transformed with certain exemplary fungal delta-9 desaturase gene sequences.

FIG. 21 includes data showing the stearic acid (C18:0) content (% FAMEs) of exemplary T₂ Arabidopsis seed from plants transformed with certain exemplary fungal delta-9 desaturase gene sequences.

FIG. 22 includes data showing the palmitoleic acid (C16:1) content (% FAMEs) of exemplary T₂ Arabidopsis seed from plants transformed with certain exemplary fungal delta-9 desaturase gene sequences.

FIG. 23 includes a graphical representation of the accumulation of HzD9DS and LnD9DS-2 mRNA transcripts (relative to AnD9DS transcripts) in developing seeds from canola plants transformed with pDAB7319 (AnD9DS v3 and LnD9DS-2 v2) or pDAB7324 (AnD9DS v3 and HzD9DS v2). The qRT-PCR ΔΔCt of each gene was determined relative to the actin transcript level, and the amount of transcript for HzD9DS and LnD9DS-2 then normalized to the level of AnD9DS transcript in each sample.

### SEQUENCE LISTING

The nucleic acid sequences listed in the accompanying sequence listing are shown using standard letter abbreviations for nucleotide bases, as defined in 37 C.F.R. §1.822. Only one strand of each nucleic acid sequence is shown, but the complementary strand is understood to be included by any reference to the displayed strand. In the accompanying sequence listing:

SEQ ID NO:1 shows a forward primer used to PCR amplify a fragment of a Magnaporthe grisea acyl-CoA delta-9 desaturase gene (referred to in some places as MgD9DS).

SEQ ID NO:2 shows a reverse primer used to PCR amplify a fragment of a M. grisea acyl-CoA delta-9 desaturase gene (referred to in some places as MgD9DS).

SEQ ID NO:3 shows an exemplary fragment of a M. grisea acyl-CoA delta-9 desaturase gene (referred to in some places as MgD9DS) that was amplified by PCR.

SEQ ID NO:4 shows an exemplary intronless MgD9DS clone.

SEQ ID NO:5 shows an exemplary nucleic acid sequence encoding a first Leptosphaeria nodorum acyl-CoA delta-9 desaturase, referred to in some places as LnD9DS-1.

SEQ ID NOs:6 and 7 show primer sequences that may be useful in some embodiments.

SEQ ID NO:8 shows an exemplary nucleic acid sequence encoding a second exemplary L. nodorum acyl-CoA delta-9 desaturase, referred to in some places as LnD9DS-2.

SEQ ID NO:9 shows a coding region from an exemplary native delta-9 desaturase gene from M. grisea (labeled as MgD9DS v1).

SEQ ID NO:10 shows a coding region from an exemplary native delta-9 desaturase gene from Helicoverpa zea (labeled as HzD9DS v1).

SEQ ID NO:11 shows a coding region from an exemplary native delta-9 desaturase (LnD9DS-2 v1) gene from L. nodorum.

SEQ ID NO:12 shows the amino acid sequence of an exemplary native delta-9 desaturase from M. grisea (MgD9DS).

SEQ ID NO:13 shows the amino acid sequence of an exemplary native delta-9 desaturase from H. zea (HzD9DS).

SEQ ID NO:14 shows the amino acid sequence of an exemplary native delta-9 desaturase from L. nodorum (LnD9DS-2).

SEQ ID NO:15 shows the sequence of an exemplary canola-optimized delta-9 desaturase gene from M. grisea (MgD9DS v2).

SEQ ID NO:16 shows the sequence of an exemplary canola-optimized delta-9 desaturase gene from H. zea (HzD9DS v2).

SEQ ID NO:17 shows the sequence of an exemplary canola-optimized delta-9 desaturase gene from L. nodorum (LnD9DS-2 v2).

SEQ ID NOs:18-39 show the sequence of primers and probes that may be useful in some embodiments.

SEQ ID NOs:40-43 show exemplary alternative Kozak sequences that may be used to increase expression in some embodiments.

SEQ ID NO:44 shows the sequence of a further exemplary canola-optimized delta-9 desaturase gene from L. nodorum (LnD9DS-2 v3).

SEQ ID NO:45 shows the sequence of a further exemplary canola-optimized delta-9 desaturase gene from H. zea (HzD9DS v3).

SEQ ID NO:46 shows the amino acid sequence of a Myc tag.

SEQ ID NO:47 shows the amino acid sequence of a HA tag.

SEQ ID NO:48 shows an exemplary nucleic acid sequence encoding an Aspergillus nidulans delta-9 desaturase, referred to in some places as AnD9DS v2.

SEQ ID NO:49 shows a second exemplary nucleic acid sequence encoding an A. nidulans delta-9 desaturase, referred to in some places as AnD9DS v3.

SEQ ID NO:50 shows the amino acid sequence encoded by nucleic acids as exemplified by SEQ ID NOs:48-49 (AnD9DS).

SEQ ID NO:51 shows the amino acid sequence of another exemplary AnD9DS desaturase.

SEQ ID NO:52 shows the amino acid sequence of an exemplary native delta-9 desaturase (ScOLE1) from Saccharomyces cerevisiae.

SEQ ID NOs:53-66 show plasmids that may be useful in some embodiments.

SEQ ID NOs:67-71 include several nucleic acid regulatory control elements that may be useful in some embodiments.

SEQ ID NO:72 shows the N-terminal 68 residues (1-68) of an exemplary AnD9DS desaturase.

SEQ ID NO:73 shows the C-terminal 175 residues (281-455) of an exemplary AnD9DS desaturase.

SEQ ID NO:74 shows a map of plasmid pDAB110110.

SEQ ID NO:75 shows a map of plasmid pDAB110112.

SEQ ID NO:76 shows an exemplary nucleic acid sequence encoding an exemplary M. grisea acyl-CoA delta-9 desaturase, referred to in some places as MgD9DS.

SEQ ID NO:77 shows an amino acid sequence comprised within the exemplary native delta-9 desaturase from L. nodorum of SEQ ID NO:14.

SEQ ID NO:78 shows an amino acid sequence comprised within the exemplary native delta-9 desaturase from H. zea of SEQ ID NO:13.

### DETAILED DESCRIPTION

### I. Overview of Several Embodiments

We previously introduced a fungal acyl-CoA delta-9 desaturase from Aspergillus nidulans into canola, thereby achieving reduced saturated fatty acid levels in seed oil. U.S. Patent Application Publication US 2008/0260933 A1. The A. nidulans delta-9 desaturase provided greater depletion of stearate (61-90%) than the more abundant palmitate fatty acids (36-49%) in the seed oil. Therefore, co-introduction of a delta-9 desaturase that acts preferentially on palmitate saturates will achieve further reductions in total saturates by complementing the stearate-preferring activity of the A. nidulans delta-9 desaturase. In some embodiments of the present invention, fungal delta-9 desaturase polypeptides having a range of substrate specificities are disclosed. Particular embodiments include a palmitate-preferring delta-9 desaturase (e.g., a native fungal enzyme as disclosed herein, or a functional equivalent thereof; and a synthetic polypeptide designed to have a preference for a palmitic acid substrate).

Disclosed herein are nucleic acid molecules encoding a delta-9 desaturase polypeptide comprising a nucleotide sequence being at least 60% identical to a sequence selected from the group consisting of SEQ ID NO:3, SEQ ID NO:4, SEQ ID NO:5, SEQ ID NO:8, SEQ ID NO:9, SEQ ID NO:10, SEQ ID NO:11, SEQ ID NO:15, SEQ ID NO:16, SEQ ID NO:17, SEQ ID NO:44, SEQ ID NO:45, SEQ ID NO:48, and SEQ ID NO:49. In some embodiments, the nucleic acid molecule may further comprises a gene regulatory element operably linked to the delta-9 desaturase polypeptide-encoding sequence. In particular embodiments, a gene regulatory element may be a phaseolin promoter, a phaseolin 5′ untranslated region, a phaseolin 3′ untranslated region, an Agrobacterium tumefaciens ORF1 3′ untranslated region, a Cassava vein Mosaic Virus promoter, a Nicotiana tabacum RB7 Matrix Attachment Region, a T-strand border sequence, a LfKCS3 promoter, and FAE 1 promoter.

Also disclosed are delta-9 desaturase polypeptides comprising an amino acid sequence being at least 80% identical to a sequence selected from the group consisting of SEQ ID NO:12, SEQ ID NO:13, SEQ ID NO:14, SEQ ID NO:50, SEQ ID NO:52, SEQ ID NO:72, and SEQ ID NO:73, as well as nucleic acid molecules encoding such delta-9 desaturase polypeptides.

In some embodiments, nucleic acid molecules and delta-9 desaturase polypeptides may be expressed in a plant material, cell, tissue, or whole plant, to decrease the amount of saturated fatty acids in the plant material, cells, tissues, or whole plants, relative to the amount observed in a wild-type plant of the same species. Alternative embodiments of the invention include methods for decreasing the amount of saturated fatty acids in the plant material, cell, tissue, or whole plant. Such methods may comprise transforming a plant material, cell, tissue, or whole plant with at least one of the aforementioned nucleic acid molecules, such that the amount of saturated fatty acids in the plant material, cell, tissue, or whole plant is decreased. Particular embodiments include methods for preferentially decreasing palmitic and/or stearic fatty acids in a plant material, cell, tissue, or whole plant.

Methods disclosed herein may be performed, for example, on plants, or plant materials derived from plants (e.g., plants of the genus Arabidopsis, or canola). A particular embodiment is drawn to methods for creating or regenerating a genetically engineered plant comprising decreased amounts of saturated fatty acids in the plant compared to a wild-type plant of the same species, the method comprising transforming a plant cell or material with at least one of the aforementioned nucleic acid molecules; and culturing the transformed plant material to obtain a plant. Plants, plant materials, plant cells, and seeds obtained by any of the aforementioned methods are al so disclosed.

### II. Abbreviations

x:yΔ^{z} fatty acid containing x carbons and y double bonds in position z counting from the carboxyl end
ACP acyl carrier protein
CoA coenzyme A
FA fatty acids
FAM fluorescein
FAS fatty acid synthase
FAME fatty acid methyl ester
KASII β-ketoacyl-ACP synthase II
MUFA monounsaturated fatty acid
WT wild type

### III. Terms

Fatty acid: As used herein, the term “fatty acid” refers to long chain aliphatic acids (alkanoic acids) of varying chain lengths, for example, from about C12 to C22, although both longer and shorter chain-length acids are known. The structure of a fatty acid is represented by the notation, x:yΔ^{z}, where “x” is the total number of carbon (C) atoms in the particular fatty acid, and “y” is the number of double bonds in the carbon chain in the position “z,” as counted from the carboxyl end of the acid.

Metabolic pathway: The term, “metabolic pathway,” refers to a series of chemical reactions occurring within a cell, catalyzed by enzymes, to achieve either the formation of a metabolic product, or the initiation of another metabolic pathway. A metabolic pathway may involve several or many steps, and may compete with a different metabolic pathway for specific reaction substrates. Similarly, the product of one metabolic pathway may be a substrate for yet another metabolic pathway.

Metabolic engineering: For the purposes of the present invention, “metabolic engineering” refers to the rational design of strategies to alter one or more metabolic pathways in a cell, such that the step-by-step modification of an initial substance into a product having the exact chemical structure desired is achieved within the overall scheme of the total metabolic pathways operative in the cell.

Desaturase: As used herein, the term “desaturase” refers to a polypeptide that can desaturate (i.e., introduce a double bond) in one or more fatty acids to produce a fatty acid or precursor of interest. A plant-soluble fatty acid desaturase enzyme may introduce a double bond regiospecifically into a saturated acyl-ACP substrate. Acyl-CoA desaturases introduce a double bond regiospecifically into a saturated fatty acyl-CoA substrate. The reaction involves activation of molecular oxygen by a two-electron reduced diiron center coordinated by a four-helix bundle that forms the core of the desaturase architecture. Of particular interest in some embodiments are acyl-CoA delta-9 desaturases.

The delta-9-18:0¹-ACP desaturase is required by all plants for the maintenance of membrane fluidity. While this enzyme primarily desaturates stearoyl-ACP, it is also active to a minor extent with palmitoyl-ACP.

Variant desaturase: As used herein, the term “variant desaturase” encompasses those desaturases that exhibit specific activity profiles consistent with a role in producing unusual fatty acids. A variant desaturase may be isolated from an organism, engineered via a directed evolution program, or engineered as a synthetic desaturase incorporating conserved amino acids from one or more characterized desaturase.

Progeny plant: For the purposes of the present invention, “progeny plant,” refers to any plant, or plant material obtained therefrom, that may be obtained by plant breeding methods. Plant breeding methods are well-known in the art, and include natural breeding, artificial breeding, selective breeding involving DNA molecular marker analysis, transgenics, and commercial breeding.

Plant material: As used herein, the term “plant material” refers to any cell or tissue obtained from a plant.

Nucleic acid molecule: A polymeric form of nucleotides, which can include both sense and anti-sense strands of RNA, cDNA, genomic DNA, and synthetic forms and mixed polymers of the above. A nucleotide refers to a ribonucleotide, deoxynucleotide, or a modified form of either type of nucleotide. A “nucleic acid molecule” as used herein is synonymous with “nucleic acid” and “polynucleotide.” The term includes single- and double-stranded forms of DNA. A nucleic acid molecule can include either or both naturally occurring and modified nucleotides linked together by naturally occurring and/or non-naturally occurring nucleotide linkages.

Nucleic acid molecules can be modified chemically or biochemically, or can contain non-natural or derivatized nucleotide bases, as will be readily appreciated by those of ordinary skill in the art. Such modification include, for example, labels, methylation, substitution of one or more of the naturally occurring nucleotides with an analog, internucleotide modifications, such as uncharged linkages (for example, methyl phosphonates, phosphotriesters, phosphoramidates, carbamates, etc.), charged linkages (for example, phosphorothioates, phosphorodithioates, etc.), pendent moieties (for example, peptides), intercalators (for example, acridine, psoralen, etc.), chelators, alkylators, and modified linkages (for example, alpha anomeric nucleic acids, etc.). The term “nucleic acid molecule” also includes any topological conformation, including single-stranded, double-stranded, partially duplexed, triplexed, hairpinned, circular and padlocked conformations.

Operably linked: A first nucleic acid sequence is operably linked with a second nucleic acid sequence when the first nucleic acid sequence is in a functional relationship with the second nucleic acid sequence. For instance, a promoter is operably linked to a coding sequence if the promoter affects the transcription or expression of the coding sequence. When recombinantly produced, operably linked nucleic acid sequences are generally contiguous and, where necessary to join two protein-coding regions, in the same reading frame. However, nucleic acids need not be contiguous to be operably linked.

Regulatory element: As used herein, the term “regulatory element” refers to a nucleic acid molecule having gene regulatory activity; i.e., one that has the ability to affect the transcription or translation of an operably-linked transcribable nucleic acid molecule. Regulatory elements such as promoters, leaders, introns, and transcription termination regions are non-coding nucleic acid molecules having gene regulatory activity which play an integral part in the overall expression of genes in living cells. Isolated regulatory elements that function in plants are therefore useful for modifying plant phenotypes through the techniques of molecular engineering. By “regulatory element,” it is intended a series of nucleotides that determines if, when, and at what level a particular gene is expressed. The regulatory DNA sequences specifically interact with regulatory proteins or other proteins.

As used herein, the term “gene regulatory activity” refers to a nucleic acid molecule capable of affecting transcription or translation of an operably linked nucleic acid molecule. An isolated nucleic acid molecule having gene regulatory activity may provide temporal or spatial expression or modulate levels and rates of expression of the operably linked nucleic acid molecule. An isolated nucleic acid molecule having gene regulatory activity may comprise a promoter, intron, leader, or 3′ transcriptional termination region.

Promoters: As used herein, the term “promoter” refers to a nucleic acid molecule that is involved in recognition and binding of RNA polymerase II or other proteins such as transcription factors (trans-acting protein factors that regulate transcription) to initiate transcription of an operably linked gene. Promoters may themselves contain sub-elements such as cis-elements or enhancer domains that effect the transcription of operably linked genes. A “plant promoter” is a native or non-native promoter that is functional in plant cells. A plant promoter can be used as a 5′ regulatory element for modulating expression of an operably linked gene or genes. Plant promoters may be defined by their temporal, spatial, or developmental expression pattern. The nucleic acid molecules described herein may comprise nucleic acid sequences comprising promoters.

Sequence identity: The term “sequence identity” or “identity,” as used herein in the context of two nucleic acid or polypeptide sequences, may refer to the residues in the two sequences that are the same when aligned for maximum correspondence over a specified comparison window.

When percentage of sequence identity is used in reference to proteins, it is recognized that residue positions which are not identical often differ by conservative amino acid substitutions, where amino acid residues are substituted for other amino acid residues with similar chemical properties (e.g., charge, hydrophobicity, or steric effects), and therefore do not change the functional properties of the molecule.

Therefore, when sequences differ by conservative substitutions, the percent sequence identity may be adjusted upwards to correct for the conservative nature of the substitution at the site of the non-identical residue. Sequences that differ by such conservative substitutions are said to have “sequence similarity” or “similarity.” Techniques for making this adjustment are well known to those of ordinary skill in the art. Typically, such techniques involve scoring a conservative substitution as a partial, rather than a full, mismatch, thereby increasing the percentage sequence identity. For example, where an identical amino acid is given a score between 0 and 1, and a non-conservative substitution is given a score of 0, a conservative substitution is given a score between 0 and 1. The scoring of conservative substitutions may be calculated, for example, as implemented in the program PC/GENE (Intelligenetics, Mountain View, Calif.).

As used herein, the term “percentage of sequence identity” may refer to the value determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity.

Analogous position in an amino acid sequence: Nucleic acid and amino acid sequences may be aligned by the methods described in the following paragraphs. When aligned, a position in one sequence is in “an analogous position” with a position in the aligned sequence if the positions are identical within the consensus sequence.

Methods for aligning sequences for comparison are well-known in the art. Various programs and alignment algorithms are described in: Smith and Waterman, Adv. Appl. math. 2:482, 1981; Needleman and Wunsch, J. Mol. Biol. 48:443, 1970; Pearson and Lipman, Proc. Natl. Acad. Sci. USA 85:2444, 1988; Higgins and Sharp, Gene 73:237-44, 1988; Higgins and Sharp, CABIOS 5:151-3, 1989; Corpet et al., Nucleic Acids Research 16:10881-10890, 1988; Huang, et al., Computer Applications in the Biosciences 8:155-65, 1992; Pearson et al., Methods in Molecular Biology 24:307-31, 1994; Tatiana et al., FEMS Microbiol. Lett., 174:247-50, 1990. Altschul et al., J. Mol. Biol. 215:403-10, 1990 (detailed consideration of sequence-alignment methods and homology calculations).

The National Center for Biotechnology Information (NCBI) Basic Local Alignment Search Tool (BLAST) is available on the Internet (at blast.ncbi.nlm.nih.gov/Blast.cgi), for use in connection with sequence-analysis programs, for example, blastp and blastn. A description of how to determine sequence identity using this program is available on the Internet through NCBI at blast.ncbi.nlm.nih.gov/Blast.cgi?CMD=Web&PAGE_TYPE=BlastDocs.

For comparisons of amino acid sequences, the “Blast 2 sequences” function of the BLAST program (bl2seq) is employed using the default parameters. Specific parameters may be adjusted within the discretion of one of skill in the art, to for example, provide a penalty for a mismatch or reward for a match.

Transformed: As used herein, the term “transformed” refers to a cell, tissue, organ, or organism into which has been introduced a foreign nucleic acid molecule, such as a construct. The introduced nucleic acid molecule may be integrated into the genomic DNA of the recipient cell, tissue, organ, or organism such that the introduced polynucleotide molecule is inherited by subsequent progeny. A “transgenic” or “transformed” cell or organism also includes progeny of the cell or organism and progeny produced from a breeding program employing such a transgenic plant as a parent in, for example, a cross and exhibiting an altered phenotype resulting from the presence of a foreign nucleic acid molecule.

### IV. Metabolic Engineering Approaches to Decreasing Saturated Fatty Acids in a Host Cell, Tissue, or Organism

A. Overview

An embodiment of the invention includes introducing delta-9 desaturases with specific acyl-CoA preferences (for example, for palmitic or stearic acid) in plant seeds. The specific acyl-CoA preference of the delta-9 desaturase enables targeting of certain specific saturated fatty acid pools (e.g., palmitate for conversion to monounsaturated products). Acyl-CoA delta-9 desaturases were selected for lowering the saturated fatty acid content in plants as they are not normally produced in plant systems to any appreciable extent.

B. Polypeptides

Polypeptides according to some embodiments of the present invention comprise an amino acid sequence showing increasing percentage identities when aligned with a sequence selected from the group consisting of SEQ ID NO:12, SEQ ID NO:13, SEQ ID NO:14, SEQ ID NO:50, SEQ ID NO:52, SEQ ID NO:72, and SEQ ID NO:73. Specific amino acid sequences within these and other embodiments may comprise sequences having, for example, at least about 70%, about 75%, about 80%, 81%, 82%, 83%, 84%, 85%, 86%, 87%, 88%, 89%, 90%, 91%, 92%, 93%, 94%, 95% 96%, 97%, 98%, 99%, or 100% identity with the aforementioned sequences. In many embodiments, the amino acid sequence having the aforementioned sequence identity when aligned with the aforementioned sequences encode a peptide with enzymatic delta-9-18:0-ACP desaturase activity, or part of a such a peptide.

C. Nucleic Acids

Some embodiments include nucleic acid molecules encoding a polypeptide described above. For example, nucleic acid sequences in some embodiments show increasing percentage identities when aligned with a sequence selected from the group consisting of SEQ ID NO:3, SEQ ID NO:4, SEQ ID NO:5, SEQ ID NO:8, SEQ ID NO:9, SEQ ID NO:10, SEQ ID NO:11, SEQ ID NO:15, SEQ ID NO:16, SEQ ID NO:17, SEQ ID NO:44, SEQ ID NO:45, SEQ ID NO:48, and SEQ ID NO:49. Specific nucleic acid sequences within these and other embodiments may comprise sequences having, for example, at least about 60%, about 65%, about 70%, about 75%, about 80%, 81%, 82%, 83%, 84%, 85%, 86%, 87%, 88%, 89%, 90%, 91%, 92%, 93%, 94%, 95% 96%, 97%, 98%, 99%, or 100% identity with a sequence selected from the group consisting of SEQ ID NO:3, SEQ ID NO:4, SEQ ID NO:5, SEQ ID NO:8, SEQ ID NO:9, SEQ ID NO:10, SEQ ID NO:11, SEQ ID NO:15, SEQ ID NO:16, SEQ ID NO:17, SEQ ID NO:44, SEQ ID NO:45, SEQ ID NO:48, and SEQ ID NO:49. It is understood by those of ordinary skill in the art that nucleic acid molecules may be modified without substantially changing the amino acid sequence of an encoded polypeptide, for example, by introducing permissible nucleotide substitutions according to codon degeneracy.

In some embodiments, nucleic acid molecules of the present invention comprise a gene regulatory element (e.g., a promoter). Promoters may be selected on the basis of the cell type into which the vector construct will be inserted. Promoters which function in bacteria, yeast, and plants are well-known in the art. The promoters may also be selected on the basis of their regulatory features. Examples of such features include enhancement of transcriptional activity, inducibility, tissue-specificity, and developmental stage-specificity. In plants, promoters that are inducible, of viral or synthetic origin, constitutively active, temporally regulated, and spatially regulated have been described. See, e.g., Poszkowski et al. (1989) EMBO J. 3:2719; Odell et al. (1985) Nature 313:810; and Chau et al. (1989) Science 244:174-81).

Useful inducible promoters include, for example, promoters induced by salicylic acid or polyacrylic acids induced by application of safeners (substituted benzenesulfonamide herbicides), heat-shock promoters, a nitrate-inducible promoter derived from the spinach nitrate reductase transcribable nucleic acid molecule sequence, hormone-inducible promoters, and light-inducible promoters associated with the small subunit of RuBP carboxylase and LHCP families.

Examples of useful tissue-specific, developmentally-regulated promoters include the β-conglycinin 7Sα promoter and seed-specific promoters. Plant functional promoters useful for preferential expression in seed plastid include those from proteins involved in fatty acid biosynthesis in oilseeds and from plant storage proteins. Examples of such promoters include the 5′ regulatory regions from such transcribable nucleic acid molecule sequences as phaseolin, napin, zein, soybean trypsin inhibitor, ACP, stearoyl-ACP desaturase, and oleosin. Another exemplary tissue-specific promoter is the lectin promoter, which is specific for seed tissue.

Other useful promoters include the nopaline synthase, mannopine synthase, and octopine synthase promoters, which are carried on tumor-inducing plasmids of Agrobacterium tumefaciens; the cauliflower mosaic virus (CaMV) 19S and 35S promoters; the enhanced CaMV 35S promoter; the Figwort Mosaic Virus 35S promoter; the light-inducible promoter from the small subunit of ribulose-1,5-bisphosphate carboxylase (ssRUBISCO); the EIF-4A promoter from tobacco (Mandel et al. (1995) Plant Mol. Biol. 29:995-1004); corn sucrose synthetase; corn alcohol dehydrogenase I; corn light harvesting compolex; corn heat shock protein; the chitinase promoter from Arabidopsis; the LTP (Lipid Transfer Protein) promoters; petunia chalcone isomerase; bean glycine rich protein 1; potato patatin; the ubiquitin promoter; and the actin promoter. Useful promoters are preferably seed-selective, tissue selective, or inducible. Seed-specific regulation is discussed in, for example, EP 0 255 378.

To obtain higher expression of a heterologous gene(s), it may be preferred to reengineer the gene(s) so that it is more efficiently expressed in the expression host cell (e.g., a plant cell, for example, canola, rice, tobacco, maize, cotton, and soybean). Therefore, an optional additional step in the design of a gene encoding a delta-9 desaturase for plant expression (i.e., in addition to the provision of one or more gene regulatory elements) is reengineering of a heterologous gene protein coding region for optimal expression. Particular embodiments include redesigned genes that have been optimized to increase the expression level (i.e. produce more protein) in a transgenic canola plant cell or Arabidopsis plant cell than in a canola plant cell or Arabidopsis plant cell transformed with the naturally-occurring heterologous gene sequence.

Due to the plasticity afforded by the redundancy/degeneracy of the genetic code (i.e., some amino acids are specified by more than one codon), evolution of the genomes in different organisms or classes of organisms has resulted in differential usage of synonymous codons. This “codon bias” is reflected in the mean base composition of protein coding regions. For example, organisms having genomes with relatively low G+C contents utilize more codons having A or T in the third position of synonymous codons, whereas those having higher G+C contents utilize more codons having G or C in the third position. Further, it is thought that the presence of “minor” codons within an mRNA may reduce the absolute translation rate of that mRNA, especially when the relative abundance of the charged tRNA corresponding to the minor codon is low. An extension of this reasoning is that the diminution of translation rate by individual minor codons would be at least additive for multiple minor codons. Therefore, mRNAs having high relative contents of minor codons in a particular expression host would have correspondingly low translation rates. This rate may be reflected by correspondingly low levels of the encoded protein.

In engineering optimized genes encoding a delta-9 desaturase for expression in canola or Arabidopsis (or other plants, such as rice, tobacco, maize, cotton or soybean), it is helpful if the codon bias of the prospective host plant(s) has been determined. Multiple publicly-available DNA sequence databases exist wherein one may find information about the codon distribution of plant genomes or the protein coding regions of various plant genes.

The codon bias is the statistical distribution of codons that the expression host (e.g., a plant such as canola or Arabidopsis) uses for coding the amino acids of its proteins. The codon bias can be calculated as the frequency at which a single codon is used relative to the codons for all amino acids. Alternatively, the codon bias may be calculated as the frequency at which a single codon is used to encode a particular amino acid, relative to all the other codons for that amino acid (synonymous codons).

In designing optimized coding regions for plant expression of delta-9 desaturase genes, the primary (“first choice”) codons preferred by the plant should be determined, as well as the second, third, fourth etc. choices of preferred codons when multiple choices exist. A new DNA sequence can then be designed which encodes the amino sequence of the delta-9 desaturase gene, wherein the new DNA sequence differs from the native DNA sequence (encoding the desaturase) by the substitution of expression host-preferred (first preferred, second preferred, third preferred, or fourth preferred, etc.) codons to specify the amino acid at each position within the amino acid sequence. The new sequence is then analyzed for restriction enzyme sites that might have been created by the modifications. The identified putative restriction sites are further modified by replacing these codons with a next-preferred codon to remove the restriction site. Other sites in the sequence which may affect transcription or translation of heterologous sequence are exon:intron junctions (5′ or 3′), poly-A addition signals, and/or RNA polymerase termination signals. The sequence may be further analyzed and modified to reduce the frequency of TA or CG doublets. In addition to these doublets, sequence blocks that have more than about six G or C nucleotides that are the same may also adversely affect transcription or translation of the sequence. Therefore, these blocks are advantageously modified by replacing the codons of first or second choice, etc. with the next-preferred codon of choice.

The method described above enables one skilled in the art to modify gene(s) that are foreign to a particular plant so that the genes are optimally expressed in plants. The method is further illustrated in PCT application WO 97/13402. Thus, optimized synthetic genes that are functionally equivalent to desaturases/genes of some embodiments may be used to transform hosts, including plants. Additional guidance regarding the production of synthetic genes can be found in, for example, U.S. Pat. No. 5,380,831.

Once a plant-optimized DNA sequence has been designed on paper or in silico, actual DNA molecules can be synthesized in the laboratory to correspond in sequence precisely to the designed sequence. Such synthetic DNA molecules may be cloned and otherwise manipulated exactly as if they were derived from natural or native sources.

D. Methods for genetic transformation of plant material

Some embodiments are directed to a method of producing a transformed cell that comprises one or more nucleic acid molecule(s) comprising a nucleic acid sequence at least 60% identical to a sequence selected from the group consisting of SEQ ID NO:3, SEQ ID NO:4, SEQ ID NO:5, SEQ ID NO:8, SEQ ID NO:9, SEQ ID NO:10, SEQ ID NO:11, SEQ ID NO:15, SEQ ID NO:16, SEQ ID NO:17, SEQ ID NO:44, SEQ ID NO:45, SEQ ID NO:48, and SEQ ID NO:49. Such nucleic acid molecules may also comprise, for example, non-coding regulatory elements, such as promoters. Other sequences may also be introduced into the cell along with the non-coding regulatory elements and transcribable nucleic acid molecule sequences. These other sequences may include 3′ transcriptional terminators, 3′ poly-adenylation signals, other untranslated sequences, transit or targeting sequences, selectable markers, enhancers, and operators.

A method of transformation generally comprises the steps of selecting a suitable host cell, transforming the host cell with a recombinant vector, and obtaining the transformed host cell. Technology for introduction of DNA into cells is well-known to those of skill in the art. These methods can generally be classified into five categories: (1) chemical methods (Graham and Van der Eb (1973) Virology 54(2):536-9; Zatloukal et al. (1992) Ann. N.Y. Acad. Sci. 660:136-53); (2) physical methods such as microinjection (Capechi (1980) Cell 22(2):479-88), electroporation (Wong and Neumann (1982) Biochim. Biophys. Res. Commun. 107(2):584-7; Fromm et al. (1985) Proc. Natl. Acad. Sci. USA 82(17):5824-8; U.S. Pat. No. 5,384,253), and particle acceleration (Johnston and Tang (1994) Methods Cell Biol. 43(A):353-65; Fynan et al. (1993) Proc. Natl. Acad. Sci. USA 90(24):11478-82; (3) viral vectors (Clapp (1993) Clin. Perinatol. 20(1):155-68; Lu et al. (1993) J. Exp. Med. 178(6):2089-96; Eglitis and Anderson (1988) Biotechniques 6(7):608-14); (4) receptor-mediated mechanisms (Curiel et al. (1992) Hum. Gen. Ther. 3(2):147-54; Wagner et al. (1992) Proc. Natl. Acad. Sci. USA 89(13):6099-103); and (5) bacterial-mediated mechanisms, such as with Agrobacterium. Alternatively, nucleic acids may be directly introduced into pollen by directly injecting a plant's reproductive organs. Zhou et al. (1983) Methods in Enzymology 101:433; Hess (1987) Intern. Rev. Cytol. 107:367; Luo et al. (1988) Plant Mol. Biol. Reporter 6:165; Pena et al. (1987) Nature 325:274. Other transformation methods include, for example, protoplast transformation as illustrated in U.S. Pat. No. 5,508,184. Nucleic acid molecules may also be injected into immature embryos. Neuhaus et al. (1987) Theor. Appl. Genet. 75:30.

The most commonly used methods for transformation of plant cells are: the Agrobacterium-mediated DNA transfer process (Fraley et al. (1983) Proc. Natl. Acad. Sci. USA 80:4803) (as illustrated in U.S. Pat. No. 5,824,877; U.S. Pat. No. 5,591,616; U.S. Pat. No. 5,981,840; and U.S. Pat. No. 6,384,301) and the biolistics or microprojectile bombardment-mediated process (i.e., the gene gun) (such as described in U.S. Pat. No. 5,550,318; U.S. Pat. No. 5,538,880; U.S. Pat. No. 6,160,208; U.S. Pat. No. 6,399,861; and U.S. Pat. No. 6,403,865). Typically, nuclear transformation is desired, but where it is desirable to specifically transform plastids, such as chloroplasts or amyloplasts, plant plastids may be transformed utilizing a microprojectile-mediated delivery of the desired nucleic acid molecule in certain plant species, such as for example, Arabidopsis, tobacco, potato, and Brassica species.

Agrobacterium-mediated transformation is achieved through the use of a genetically engineered soil bacterium belonging to the genus Agrobacterium. Several Agrobacterium species mediate the transfer of a specific DNA known as “T-DNA,” which can be genetically engineered to carry any desired piece of DNA into many plant species. The major events marking the process of T-DNA mediated pathogensis are: induction of virulence genes, and processing and transfer of T-DNA. This process is the subject of many reviews. See, e.g., Ream (1989) Ann. Rev. Phytopathol. 27:583-618; Howard and Citovsky (1990) Bioassays 12:103-8; Kado (1991) Crit. Rev. Plant Sci. 10:1-32; Zambryski (1992) Annual Rev. Plant Physiol. Plant Mol. Biol. 43:465-90; Gelvin (1993) in Transgenic Plants, Kung and Wu eds., Academic Press, San Diego, Calif., pp. 49-87; Binns and Howitz (1994) In Bacterical Pathogenesis of Plants and Animals, Dang, ed., Berlin: Springer Verlag., pp. 119-38; Hooykaas and Beijersbergen (1994) Ann. Rev. Phytopathol. 32:157-79; Lessl and Lanka (1994) Cell 77:321-4; and Zupan and Zambryski (1995) Annual Rev. Phytopathol. 27:583-618.

To select or score for transformed plant cells regardless of transformation methodology, the DNA introduced into the cell may contain a gene that functions in a regenerable plant tissue to produce a compound that confers upon the plant tissue resistance to an otherwise toxic compound. Genes of interest for use as a selectable, screenable, or scorable marker include, but are not limited to, β-glucuronidase (GUS), green fluorescent protein (GFP), luciferase, and antibiotic or herbicide tolerance genes. Examples of antibiotic resistance genes include genes conferring resistance to the penicillins, kanamycin (and neomycin, G418, bleomycin); methotrexate (and trimethoprim); chloramphenicol; and tetracycline. For example, glyphosate resistance may be conferred by a herbicide resistance gene. Della-Cioppa et al. (1987) Bio/Technology 5:579-84. Other selection devices can also be implemented, including for example and without limitation, tolerance to phosphinothricin, bialaphos, and positive selection mechanisms (Joersbro et al. (1998) Mol. Breed. 4:111-7), and are considered within the scope of embodiments of the present invention.

The transformed cells, identified by selection or screening and cultured in an appropriate medium that supports regeneration, may then be allowed to mature into plants.

The presently disclosed methods may be used with any transformable plant cell or tissue. Transformable cells and tissues, as used herein, includes but is not limited to those cells or tissues that are capable of further propagation to give rise to a plant. Those of skill in the art recognize that a number of plant cells or tissues are transformable in which after insertion of exogenous DNA and appropriate culture conditions the plant cells or tissues can form into a differentiated plant. Tissue suitable for these purposes can include but is not limited to immature embryos, scutellar tissue, suspension cell cultures, immature inflorescence, shoot meristem, nodal explants, callus tissue, hypocotyl tissue, cotyledons, roots, and leaves.

The regeneration, development, and cultivation of plants from transformed plant protoplast or explants are known in the art. Weissbach and Weissbach (1988) Methods for Plant Molecular Biology, (Eds.) Academic Press, Inc., San Diego, Calif.; Horsch et al. (1985) Science 227:1229-31. This regeneration and growth process typically includes the steps of selecting transformed cells and culturing those cells through the usual stages of embryonic development through the rooted plantlet stage. Transgenic embryos and seeds are similarly regenerated. In this method, transformants are generally cultured in the presence of a selective media which selects for the successfully transformed cells and induces the regeneration of plant shoots. Fraley et al. (1993) Proc. Natl. Acad. Sci. USA 80:4803. These shoots are typically obtained within two to four months. The resulting transgenic rooted shoots are thereafter planted in an appropriate plant growth medium such as soil. Cells that survive the exposure to a selective agent, or cells that have been scored positive in a screening assay, may be cultured in media that supports regeneration of plants. The shoots may then be transferred to an appropriate root-inducing medium containing the selective agent and an antibiotic to prevent bacterial growth. Many of the shoots will develop roots. These are then transplanted to soil or other media to allow the continued development of roots. The method, as outlined above, will generally vary depending on the particular plant strain employed, and particulars of the methodology are therefore within the discretion of one of skill in the art.

The regenerated transgenic plants may be self-pollinated to provide homozygous transgenic plants. Alternatively, pollen obtained from the regenerated transgenic plants may be crossed with non-transgenic plants, preferably inbred lines of agronomically important species. Conversely, pollen from non-transgenic plants may be used to pollinate the regenerated transgenic plants.

The transgenic plant may pass along the transformed nucleic acid sequence to its progeny. The transgenic plant is preferably homozygous for the transformed nucleic acid sequence and transmits that sequence to all of its offspring upon, and as a result of, sexual reproduction. Progeny may be grown from seeds produced by the transgenic plant. These additional plants may then be self-pollinated to generate a true breeding line of plants.

The progeny from these plants may be evaluated, among other things, for gene expression. The gene expression may be detected by several common methods such as western blotting, northern blotting, immunoprecipitation, and ELISA (Enzyme-Linked ImmunoSorbent Assay). The transformed plants may also be analyzed for the presence of the introduced DNA and the expression level and/or fatty acid profile conferred by the nucleic acid molecules and amino acid molecules of the present invention. Those of skill in the art are aware of the numerous methods available for the analysis of transformed plants. For example, methods for plant analysis include, but are not limited to, Southern blots or northern blots, PCR-based approaches, biochemical assays, phenotypic screening methods, field evaluations, and immunodiagnostic assays.

Methods for specifically transforming dicots are well-known to those skilled in the art. Transformation and plant regeneration using these methods have been described for a number of crops including, but not limited to, members of the genus Arabidopsis, cotton (Gossypium hirsutum), soybean (Glycine max), peanut (Arachis hypogaea), and members of the genus Brassica. Methods for transforming dicots, primarily by use of Agrobacterium tumefaciens, and obtaining transgenic plants have been published for cotton (U.S. Pat. No. 5,004,863; U.S. Pat. No. 5,159,135; U.S. Pat. No. 5,518,908); soybean (U.S. Pat. No. 5,569,834; U.S. Pat. No. 5,416,011; McCabe et al. (1988) Biotechnology 6:923; Christou et al. (1988) Plant Physiol. 87:671-4); Brassica (U.S. Pat. No. 5,463,174); peanut (Cheng et al. (1996) Plant Cell Rep. 15:653-7; McKently et al. (1995) Plant Cell Rep. 14:699-703); papaya; and pea (Grant et al. (1995) Plant Cell Rep. 15:254-8).

Methods for transforming monocots are also well-known in the art. Transformation and plant regeneration using these methods have been described for a number of crops including, but not limited to, barley (Hordeum vulgarae); maize (Zea mays); oats (Avena sativa); orchard grass (Dactylis glomerata); rice (Oryza sativa, including indica and japonica varieties); sorghum (Sorghum bicolor); sugar cane (Saccharum sp); tall fescue (Festuca arundinacea); turfgrass species (e.g., Agrostis stolonifera, Poa pratensis, Stenotaphrum secundatum); wheat (Triticum aestivum); and alfalfa (Medicago sativa). It is apparent to those of skill in the art that a number of transformation methodologies can be used and modified for production of stable transgenic plants for any number of target crops of interest.

Any plant may be chosen for use in the presently disclosed methods. Preferred plants for modification according to the present invention include, for example and without limitation, oilseed plants, Arabidopsis thaliana, borage (Borago spp.), canola (Brassica spp.), castor (Ricinus communis), cocoa bean (Theobroma cacao), corn (Zea mays), cotton (Gossypium spp), Crambe spp., Cuphea spp., flax (Linum spp.), Lesquerella and Limnanthes spp., Linola, nasturtium (Tropaeolum spp.), Oenothera spp., olive (Olea spp.), palm (Elaeis spp.), peanut (Arachis spp.), rapeseed, safflower (Carthamus spp.), soybean (Glycine and Soja spp.), sunflower (Helianthus spp.), tobacco (Nicotiana spp.), Vernonia spp., wheat (Triticum spp.), barley (Hordeum spp.), rice (Oryza spp.), oat (Avena spp.) sorghum (Sorghum spp.), and rye (Secale spp.) or other members of the Gramineae.

It is apparent to those of skill in the art that a number of transformation methodologies can be used and modified for production of stable transgenic plants from any number of target crops of interest.

E. Transgenic Seeds

In some embodiments, a transgenic seed may comprise a delta-9 desaturase polypeptide comprising an amino acid sequence being at least 80% identical to a sequence selected from the group consisting of SEQ ID NO:12, SEQ ID NO:13, SEQ ID NO:14, SEQ ID NO:50, SEQ ID NO:51, SEQ ID NO:52, SEQ ID NO:72, and SEQ ID NO:73. In these and other embodiments, the transgenic seed may comprise a nucleic acid sequence being at least 60% identical to a sequence selected from the group consisting of SEQ ID NO:3, SEQ ID NO:4, SEQ ID NO:5, SEQ ID NO:8, SEQ ID NO:9, SEQ ID NO:10, SEQ ID NO:11, SEQ ID NO:15, SEQ ID NO:16, SEQ ID NO:17, SEQ ID NO:44, SEQ ID NO:45, SEQ ID NO:48, and SEQ ID NO:49. In certain embodiments, a transgenic seed may exhibit decreased levels of saturated fatty acids (for example, palmitic fatty acids and/or stearic fatty acids). The seeds may be harvested from a fertile transgenic plant, and may be used to grow progeny generations of transformed plants, including hybrid plant lines comprising at least one nucleic acid sequence as set forth above, and optionally at least one additional gene or nucleic acid construct of interest.

Each document, patent, and reference cited herein is herein incorporated by its entirety.

The following examples are provided to illustrate certain particular features and/or embodiments. These examples should not be construed to limit the invention to the particular features or embodiments described.

### EXAMPLES

### Example I: Cloning of Acyl-CoA Delta-9 Desaturases and Functional Characterization in Ole1-Deficient Yeast

Cloning of Magnaporthe grisea Acyl-CoA Delta-9 Desaturases

The Magnaporthe grisea acyl-CoA delta-9 desaturase gene (MgD9DS) was isolated from genomic DNA using primers based on a published NCBI/Broad Institute sequence originally annotated as a “hypothetical protein,” and having 55.4% identity at the nucleotide level to the S. cerevisiae acyl-CoA delta-9 desaturase (i.e., OLE1). Forward and reverse primers, each 41 base pairs in length, were designed. The forward primer, MgΔ9F (SEQ ID NO:1), included an EcoRI site at the 5′ end. The reverse primer, Mg9ΔR (SEQ ID NO:2), contained stop codons in each of three reading frames and a terminal XhoI site.

The MgD9DS gene was PCR amplified using the Takara EZ Taq™ PCR kit (Takara Bio Inc., Otsu, Shiga, Japan) following the manufacturer's protocol. The amplification conditions were 94° C. for 1 minute, followed by 30 cycles of 94° C. for 30 sec, 60° C. for 60 seconds, and an extension at 72° C. for 90 seconds. A final extension step was performed at 72° C. for 10 minutes. The expected 1,425 base pair PCR product was excised from an agarose gel and purified using Montage spin columns per manufacturer's recommendations (Millipore, Billerica, Mass.). The purified fragment was cloned into the pCR® 2.1 TOPO® cloning vector (Invitrogen, Carlsbad, Calif.). The TOPO reaction was transformed into chemically competent Top 10 E. coli cells per supplier conditions. Bacterial colonies containing the putative clone were isolated. Mini-plasmid preps were preformed with a Macherey-Nagel Nucleospin DNA isolation kit (Machery-Nagel, Neumann-Neander-Strasse, Düren, Germany), and DNA was digested with EcoRI and XhoI restriction enzymes. Positive clones containing the expected 1,425 bp MgD9DS gene fragment were identified. The nucleotide sequence was obtained via sequencing reactions. The sequence of the PCR amplified fragment is listed as SEQ ID NO:3.

Sequence analysis revealed a small (90 bp) intron located in the 5′ end of the MgD9DS gene. The intron was removed using Splice Overlap Extension PCR. The resulting PCR amplicon was gel purified, cloned into the pCR® 2.1 TOPO® cloning vector, and transformed into Top 10 E. coli cells. Several clones were identified via analysis of restriction enzyme digests of purified DNA from single transformant colonies. These clones were sequenced to confirm the presence of an intronless MgD9DS clone. The resulting sequence is listed as SEQ ID NO:4.

The MgD9DS genes, with and without the intron, were each subcloned as an EcoRI/XhoI fragment into a yeast expression vector. This yeast expression vector contains an Aspergillus nidulans delta-9 desaturase (AnD9DS) gene flanked by the S. cerevisiae delta-9 desaturase promoter and delta-9 desaturase 3′UTR/terminator. The Aspergillus nidulans delta-9 desaturase gene was excised on an EcoRI/XhoI fragment, which was replaced with either the MgD9DS gene-containing fragment or the intronless MgD9DS gene-containing fragment. Two clones containing the MgD9DS gene (one with an intron, and one without an intron) were advanced for S. cerevisiae transformation.

A delta-9 desaturase deficient S. cerevisiae strain (OFY093), which is maintained on Yeast Peptone Dextrose (YPD) media with Tween® 80, was transformed using the Alkali-Cation Yeast Transformation Kit (Qbiogene Montreal, Canada). Complemented strains were identified by growth on media that did not contain Tween® 80 (monounsaturated fatty acid supplement) or uracil (Dropout Base with Agar with SC-URA). Complemented strains were single colony purified on selective media three times. Complemented strains were further verified by PCR amplification of the delta-9 desaturase gene, and sequencing of the PCR product. In addition, strains containing the MgD9DS clone were reverted to fatty acid and uracil dependence by passing the strain at least three times on YPD+Tween 80® media, then patching strains to DOBA SC-URA minus Tween® 80 media.

Expression of the intron-containing MgD9DS coding sequence was unsuccessful, indicating that the intron was not spliced by the yeast machinery. The substrate specificity of the yeast strain containing the intronless MgD9DS coding sequence was further characterized by FAME analysis.

Cloning of Leptosphaeria nodorum Acyl-CoA Delta-9 Desaturases

Two Leptosphaeria nodorum EST sequences (1,246 and 429 base pairs, respectively) were identified from a collection of L. nodorum ESTs by using a BlastN search as sharing high levels of sequence identity (54.0% and 54.2% respectively) with the S. cerevisiae acyl-CoA delta-9 desaturase (OLE1). When aligned, these sequences were 64.6% identical to one another, suggesting the presence of two distinct Leptosphaeria nodorum acyl-CoA delta-9 desaturases. An LnD9DS-1 gene (SEQ ID NO:5) was isolated by screening a L. nodorum cDNA library with the 1,246 bp gene probe. The sequence of this gene was obtained, and the coding sequence was isolated. The entire sequence of an LnD9DS-2 gene was isolated by first BLAST searching the published Broad Institute Leptosphaeria nodorum genome sequence with the 429 bp EST sequence. This search identified Supercontig Ln 1.4 as containing a gene with 100% homology to the 429 bp fragment, which gene was annotated as encoding a “hypothetical protein.” Next, the LnD9DS-2 gene was cloned from a Leptosphaeria nodorum cDNA library using PCR primers based on the Ln1.4 supercontig sequence. The primer sequences used were Lnd9FAD2F (SEQ ID NO:6) and Lnd9FAD2R (SEQ ID NO:7). The forward primer was designed with a 5′ BamHI site, and the reverse primer contained stop codons in three reading frames and a terminal NcoI site.

An aliquot of the Leptosphaeria nodorum cDNA library was diluted 1/10 to provide 400 ng of template DNA for the PCR reaction. PCR amplification was performed using a Takara EZ Taq™ PCR kit following the recommended amplification conditions of 94° C. for 1 minute, followed by 30 cycles of 94° C. for 30 seconds, 60° C. for 60 seconds, and extension at 72° C. for 90 seconds. A final extension step was performed at 72° C. for 10 minutes. The expected 1,370 base pair product was excised from an agarose gel, and purified using Montage spin columns per the manufacturer's recommendations. The purified fragment was cloned into the pCR® 2.1 TOPO® cloning vector. The ligation reaction was transformed into chemically competent Top 10 E. coli cells according to the manufacturer's recommended protocol. Colonies containing a putative clone were isolated. Mini plasmid preps were preformed with Macherey-Nagel Nucleospin columns, and DNA was digested with BamHI and NcoI restriction enzymes. Putative LnD9DS-2 clones were identified and sequenced.

Upon sequencing, a clone of LnD9DS-2 (SEQ ID NO:8) was confirmed by comparison with the “hypothetical protein” sequence. A conservative change in the sequence of LnD9DS-2 was identified. The codon TGC (cysteine) was changed to AGC (serine) by substitution of an adenine for a thymidine at base position 271, which codon is translated to amino acid 89 of the published sequence. This is a conservative change, and the cysteine is not found to be a highly conserved amino acid among multiple filamentous fungi, so no correction was attempted.

The LnD9DS-1 and LnD9DS-2 genes of SEQ ID NOs:5 and 8, respectively, were cloned into a yeast expression vector. Clones containing either of the LnD9DS-1 and LnD9DS-2 coding sequences were confirmed by restriction enzyme analysis and DNA sequencing.

A delta-9 desaturase deficient S. cerevisiae strain (OFY093), which is maintained on YPD media with Tween® 80, was transformed using the Alkali-Cation Yeast Transformation Kit from Qbiogene. Complemented strains were identified by growth on media that did not contain Tween® 80 (monounsaturated fatty acid supplement) or uracil (DOBA sc-ura). The complemented strains were single colony purified on selective media three times. Complemented strains were further verified by PCR amplification of the delta-9 desaturase gene and sequencing of the PCR product. In addition, strains containing a LnD9DS-2 clone were reverted to fatty acid and uracil dependence by passing each strain at least three times on YPD+Tween® 80 media, then patching strains to DOBA SC-URA minus Tween® 80 media. The substrate specificities of the yeast strains containing either the LnD9DS-1 or LnD9DS-2 coding sequence were further characterized by FAME analysis.

Cloning and Transformation of Delta-9 Desaturase Deficient S. cerevisiae with HzD9DS Gene

A plant-optimized synthetic gene encoding the Helicoverpa zea acyl-CoA delta-9 desaturase (HzD9DS) (identified as HzPGDS2 in Rosenfield et al. (2001) Insect Biochem. Mol. Biol. 31(10):949-64) was excised from DASPICO89 (described below) on a BamHI/XhoI fragment and gel purified using Montage spin columns. This fragment was ligated into corresponding restriction enzyme sites of a yeast expression vector described previously and transformed into E. coli strain DH5α using standard molecular biology techniques and supplier protocols (Invitrogen, Carlsbad, Calif.).

Following restriction analysis and DNA sequencing, a clone containing the HzD9DS gene was selected for transformation into the delta-9 desaturase deficient S. cerevisiae strain, OFY093. The OFY093 strain, which is maintained on YPD media with Tween® 80, was transformed using the Alkali-Cation Yeast Transformation Kit from Qbiogene. Complemented strains were identified by growth on media that did not contain Tween® 80 (fatty acid supplement) and uracil (DOBA SC-URA). Putative complemented strains were single colony purified on selective media three times. Complemented strains were further verified by: i) extraction of plasmid DNA, using the Qbiogene Yeast plasmid purification kit, followed by PCR amplification using HzD9DS gene-specific primers; ii) sequencing of the HzD9DS gene-specific PCR product; and iii) reversion of the strain to fatty acid and URA-3 dependence by passing the strain at least three times on YPD+Tween® 80 media, then patching strains to DOBA SC-URA minus Tween® 80 media. The substrate specificity of one verified complemented HzD9DS yeast strain was further characterized by FAME analysis.

Analysis of LnD9DS-1, LnD9DS-2, MgD9DS, and HzD9DS Expressed in OLE1-Deficient Yeast Strain

As set forth, supra, three exemplary acyl-CoA delta-9 desaturase (D9DS) genes were cloned from the plant pathogenic fungi, Magnaporthe grisea (MgD9DS) and Leptosphaeria nodorum (LnD9DS-1 and LnD9DS-2). These genes and their encoded proteins have not been previously characterized. Acyl-CoA delta-9 desaturases catalyze the formation of a cis double bond between carbon atoms 9 and 10 of saturated 14-, 16-, and 18-carbon fatty acyl thioesters of Coenzyme A, resulting in production of myristoleic (14:1), palmitoleic (16:1), or oleic acid (18:1), respectively. Effects related to organism-specific biology are eliminated by expressing the different fungal acyl-CoA delta-9 desaturase genes in the same biological context. Expression of the fungal acyl-CoA delta-9 desaturase genes was therefore driven using the endogenous ole1 gene promoter within a palmitoyl-stearoyl CoA desaturase (OLE1)-deficient OFY093 yeast strain. Thus, observed differences in fatty acid substrate specificity in this system are attributable to the specific fungal delta-9 desaturase expressed in the complemented S. cerevisiae strain.

The substrate specificities of the MgD9DS, LnD9DS-1 and LnD9DS-2 CoA desaturases expressed in the complemented OYF093 strains were characterized and compared to OFY093 complemented with the AnD9DS (sdeA) described in WO/1999/050430. A yeast expression construct containing the AnD9DS gene, expression of which is driven by the ole1 gene promoter, was transformed into the S. cerevisiae OFY093 strain and expressed using the protocol described above.

The complemented S. cerevisiae strains were grown in minimal media with no fatty acid supplementation at 30° C. for 24 hours. Quantitative FAME analysis was performed on washed and lyophilized cell pellets. The results of this analysis are shown in Table 1. LnD9DS-2 promotes formation of C14:1 and C16:1, whereas LnD9DS-1 and MgD9DS have a preference for C18:0, as indicated by the ratio of C16:1/18:1 fatty acids in the yeast fatty acid compositional analyses.

TABLE 1
Comparison of fatty acid composition of ole1-deficient yeast expressing four different
fungal desaturases

| | | | | | | | C16:1/ | C18:1/ | C16:1/ |
|---|---|---|---|---|---|---|---|---|---|
| Desaturase | C14:0 | C14:1 | C16:0 | C16:1 | C18:0 | C18:1 | 16:0 | 18:0 | 18:1 |
| LnD9DS-1 | 1.5 | 0.0 | 36.5 | 8.7 | 1.8 | 51.5 | 0.2 | 28.2 | 0.17 |
| LnD9DS-2 | 1.0 | 0.1 | 26.6 | 38.1 | 6.3 | 27.9 | 1.4 | 4.4 | 1.37 |
| AnD9DS | 0.5 | 0.0 | 26.3 | 7.8 | 2.0 | 63.4 | 0.3 | 31.7 | 0.12 |
| MgD9DS | 0.5 | 0.0 | 22.7 | 9.1 | 1.8 | 65.9 | 0.4 | 37.0 | 0.14 |
| wild type yeast | 0.6 | 0.0 | 9.6 | 38.6 | 6.9 | 44.3 | | | |
| ole1-null + | 2.6 | 0.4 | 38.0 | 10.9 | 7.8 | 40.4 | | | |
| Tween ® 80 | | | | | | | | | |
| Empty vector + | 2.2 | 0.3 | 40.3 | 8.7 | 8.7 | 39.8 | | | |
| Tween ® 80 | | | | | | | | | |

The novel desaturases were further compared to the native S. cerevisiae stearoyl-CoA delta-9 desaturase (ole1) transferred into the same recombinant expression environment. A yeast expression vector containing the nucleotide sequence from S. cerevisiae described in WO/2000/011012 was constructed. The yeast expression construct containing the native S. cerevisiae stearoyl-CoA delta-9 desaturase was transformed into the S. cerevisiae OFY093 strain and expressed using the protocol described above. Another non-fungal acyl-CoA delta-9 desaturase from the insect species, Helicoverpa zea (HzD9DS), was also evaluated in these experiments.

Complemented S. cerevisiae strains containing one of the MgD9DS, LnD9DS-2 and HzD9DS genes were grown in Drop Out Broth SC-URA. A control strain, pDAB467EV-1 (pDAB467B/N transformed into OFY093 by previously described Yeast Transformation methodology), was grown in DOB SC-URA+Tween® 80, and the parent delta-9 desaturase-deficient S. cerevisiae strain, OFY093, was grown in DOB scAA+Tween® 80. Cultures were inoculated with a loop of cells from a fresh streak plate of the same media containing 1.5% agar. Strains were grown at 30° C. for 24 hours. Cultures were spun at 6,000 rpm for 10 minutes. Pellets were washed in water, spun again at 6,000 rpm for 10 minutes, and then frozen at −20° C. until FAME analysis was performed. Three sets of expression cultures were analyzed.

Freeze-dried yeast pellets were saponified in methanol containing 10% (w/v) NaOH. Nonsaponifiable lipid contaminants (sterols) were removed with hexane. The methanol fraction was acidified by addition of H₂SO₄, and the protonated fatty acids were extracted with hexane. The isolated hexane fraction was dried down, and fatty acids were methylated with 0.5 N MeOHCl at 80° C. for 30 minutes. The resulting FAMEs were extracted with hexane containing undecanoate methyl ester as an internal standard. The FAME extracts were analyzed with a HP6890 Gas Chromatograph-Flame Ionization Detector (Santa Clara, Calif.) equipped with a capillary column BPX 70 (15 m×0.25 mm×0.25 μm) from SGE (Austin, Tex.). FAMEs were separated in a temperature gradient using helium as the carrier gas. Each FAME species was identified by retention time, and quantified by the injection of a FAME rapeseed oil reference mix from Matreya, LLC (Pleasant Gap, Pa.), as the calibration standard.

Table 2 shows the fatty acid composition (as % FAMEs) of ole1-deficient OFY093 yeast cells expressing various exemplary acyl Co-A delta-9 desaturases. All strains grew well and were fully-complemented by the introduced desaturases without any requirement for exogenous MUFAs (monounsaturated fatty acids).

TABLE 2
Fatty acid composition (as % Total FAMEs) of ole1-deficient yeast strain OFY093
expressing acyl Co-A delta-9 desaturases. (Standard Deviation is in parentheses).

| Desaturase | n | C14:0 | C14:1 | C16:0 | C16:1 | C18:0 | C18:1 |
|---|---|---|---|---|---|---|---|
| LnD9DS-2 | 7 | 1.4 (0.7) | 1.4 (1.0) | 26.6 (4.5) | 38.8 (2.8) | 6.0 (1.3) | 25.4 (4.4) |
| HzD9DS | 6 | 2.6 (1.3) | 0.9 (0.5) | 34.7 (6.8) | 37.5 (4.2) | 6.0 (1.1) | 18.4 (4.1) |
| ole1 | 6 | 1.1 (0.4) | 0.6 (0.4) | 14.4 (2.6) | 49.2 (1.6) | 5.6 (1.1) | 24.0 (1.1) |
| AnD9DS | 8 | 0.5 (0.3) | 0.2 (0.2) | 23.5 (2.2) | 9.3 (3.0) | 2.1 (0.5) | 64.6 (3.2) |
| MgD9DS | 2 | 0.9 (0.0) | 0.1 (0.0) | 21.2 (0.2) | 12.1 (0.1) | 1.6 (0.1) | 64.2 (0.3) |

These data show that the fatty acid composition of the complemented yeast strains varies according to the introduced gene. LnD9DS-2 produces relatively high amounts of C16:1, as does HzD9DS and ole1, whereas AnD9DS and MgD9DS produce relatively high amounts of C18:1.

The differential level of conversion based upon chain length can be further shown by calculating the proportion of MUFA relative to the total fatty acids for each fatty acid chain length; C14, C16, or C18. These data show the relatively high conversion to C16:1 for LnD9DS-2 and HzD9DS, and to C18:1 for AnD9DS and MgD9DS. Table 3. The bottom four rows represent control samples complemented with added tergitol, unsaturated fatty acids, or Tween®. Samples with different letters are significantly different, as determined via the Tukey-Kramer Test performed in the JMP statistical software suite (SAS Institute Inc., Cary, N.C.).

TABLE 3
Proportion of MUFA of total fatty acids for
each chain length (Cxx:1/(Cxx:0 + Cxx:1).

| Desaturase | C14 | C16* | C18 |
|---|---|---|---|
| LnD9DS-2 | 0.49 | 0.60 (b) | 0.81 (b) |
| HzD9DS | 0.30 | 0.52 (b) | 0.75 (c) |
| ole1 | 0.34 | 0.79 (a) | 0.81 (b) |
| AnD9DS | 0.25 | 0.28 (c) | 0.97 (a) |
| MgD9DS | 0.07 | 0.36 (c) | 0.98 (a) |
| None + tergitol | 0.06 | 0 | 0 |
| None + tergitol + ricinoleic | 0.07 | 0 | 0.01 |
| None + tergitol + linoleic | 0 | 0 | 0.04 |
| None + tween | 0.65 | 0.23 | 0.87 |

*C16 MUFA includes cis-vaccenic acid (C18:1 Δ11), as it is derived from elongation of palmitoleic acid (C16:1 Δ9).

Phylogeny of Fungal Acyl-CoA Desaturases

Phylogenetic analysis of multiple fungal acyl-CoA delta-9 desaturase amino acid sequences suggests that LnD9DS-2 is distinct from the 18:0-preferring delta-9 desaturases. Thus, we hypothesized that characterization of other fungal delta-9 desaturases closely associated with either the 18:0-preferring delta-9 desaturases, or with LnD9DS-2, may identify desaturases with a range of 18:0 or 16:0 activities. Our hypothesis predicts that a fungal delta-9 desaturase that is more closely associated with LnD9DS-2 will have increased 16:0 activity.

A search of the public DNA sequence databases (Broad Institute, NCBI, etc.) did not identify any gene sequences specifically annotated as delta-9 desaturases in Magnaporthe grisea or Leptosphaeria nodorum. Pfam analysis of the Broad Institute sequences that were identified within this disclosure indicates that these proteins contain cytochrome B5 and desaturase motifs that are also found in other fungal acyl-CoA delta-9 desaturases. However, the proteins had not been previously identified as acyl-CoA delta-9 desaturases. We have demonstrated this function of these proteins by complementation in yeast, reversal studies, and DNA sequence analysis.

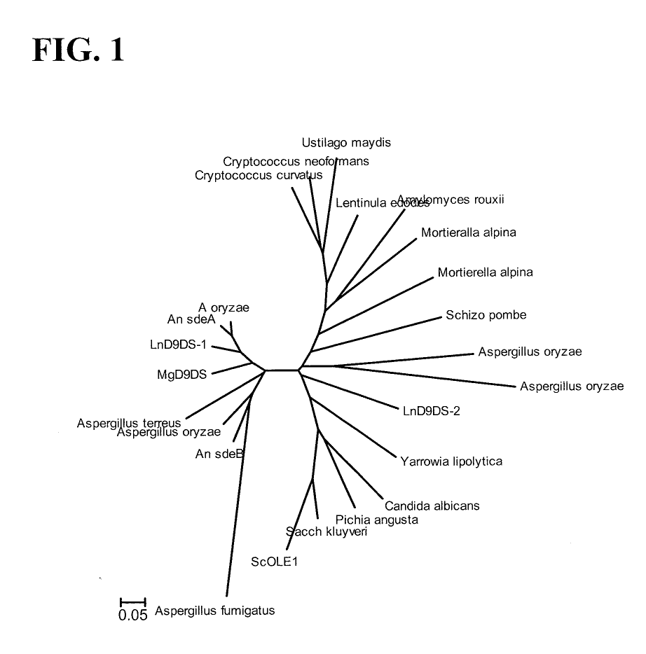

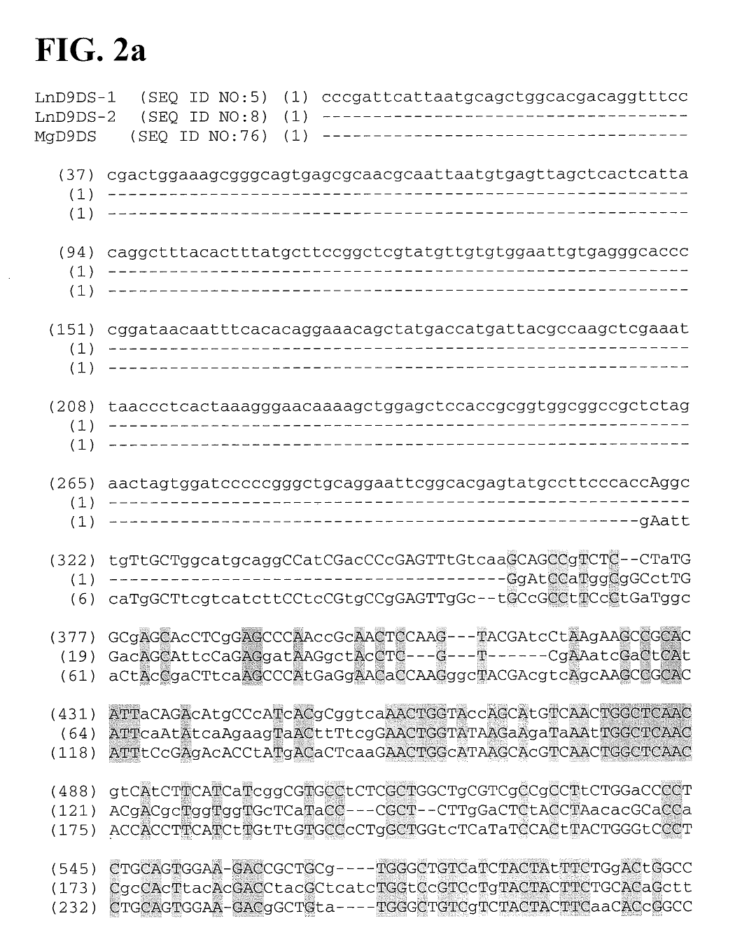

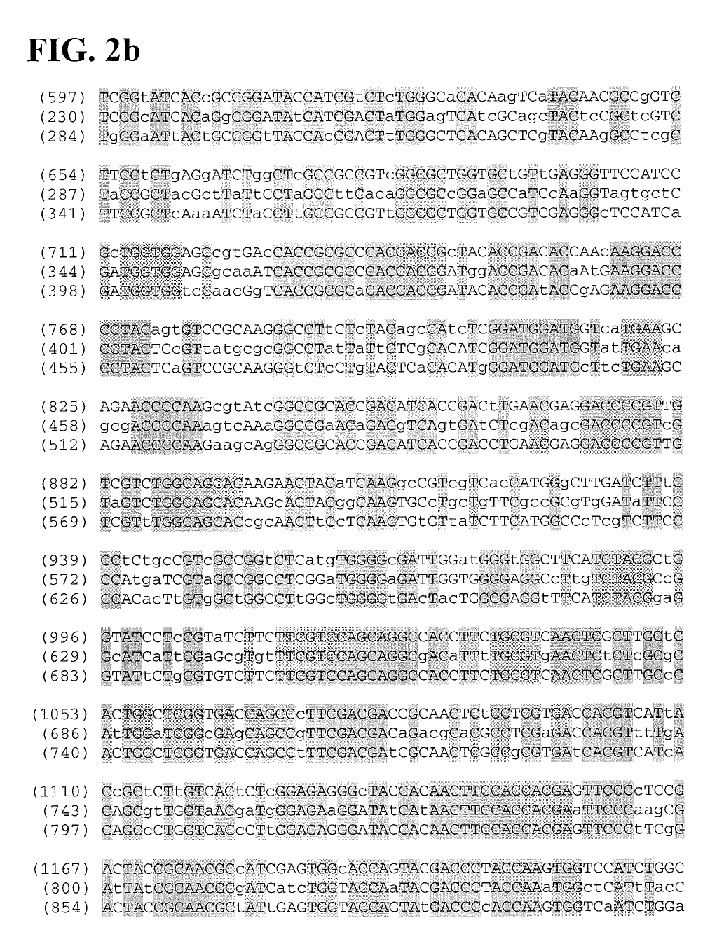

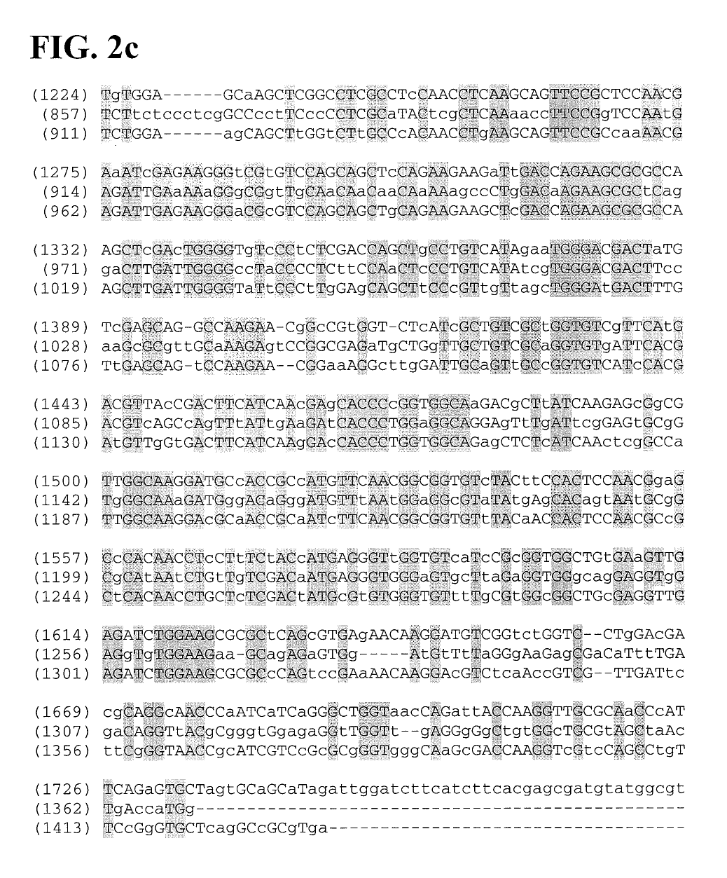

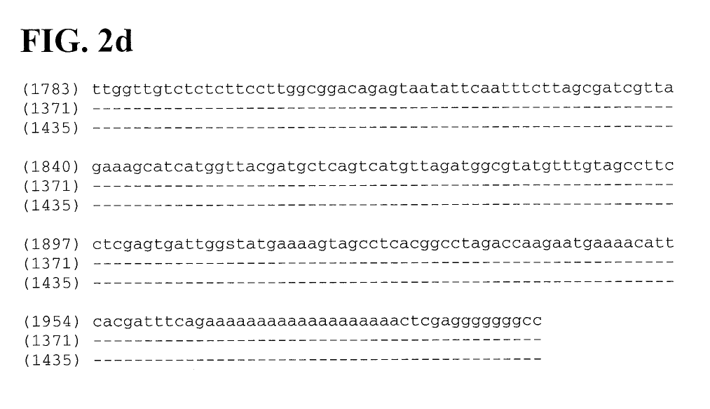

The relationships of several fungal desaturase gene sequences were analyzed phylogenetically using the neighbor-joining method via the MEGA software package. Tamura et al. (2007) Mol. Biol. and Evolution 24:1596-9. FIG. 1 illustrates this phylogenetic analysis of the fungal desaturase sequences. These sequences were recovered by BlastN searches of the NCBI sequence database using the AnD9DS (sdeA) amino acid sequence. LnD9DS-1 and MgD9DS share higher levels of sequence identity with one another, as compared to LnD9DS-2. Additionally, a ClustalW alignment of LnD9DS-1, LnD9DS-2, and MgD9DS shows the divergence of LnD9DS-2 from LnD9DS-1 and MgD9DS. FIG. 2. The nucleotide sequences of LnD9DS-1 and MgD9DS share a higher number of base pairs in common.

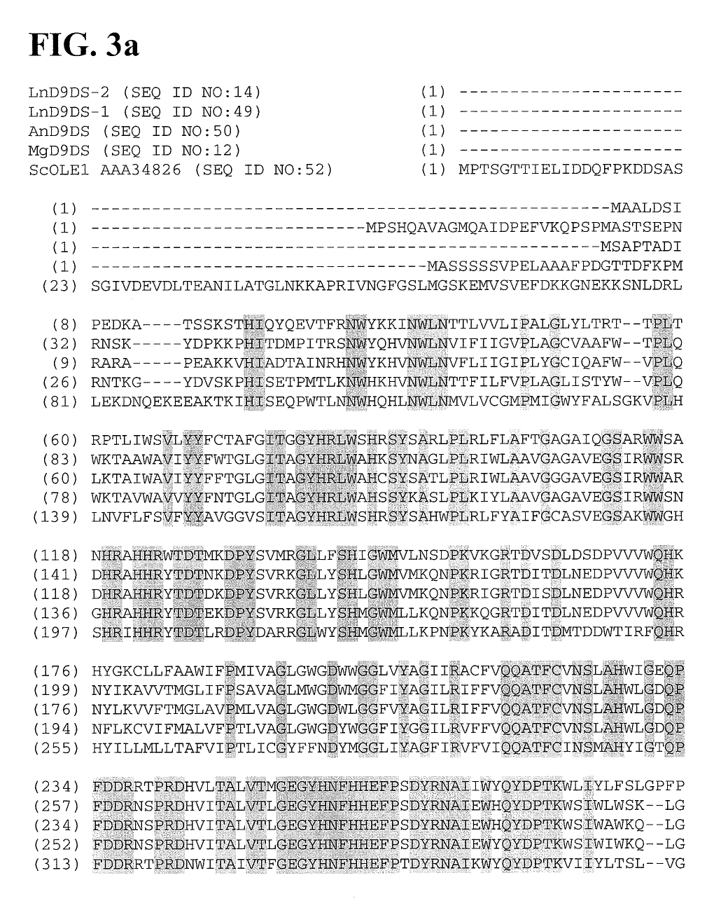

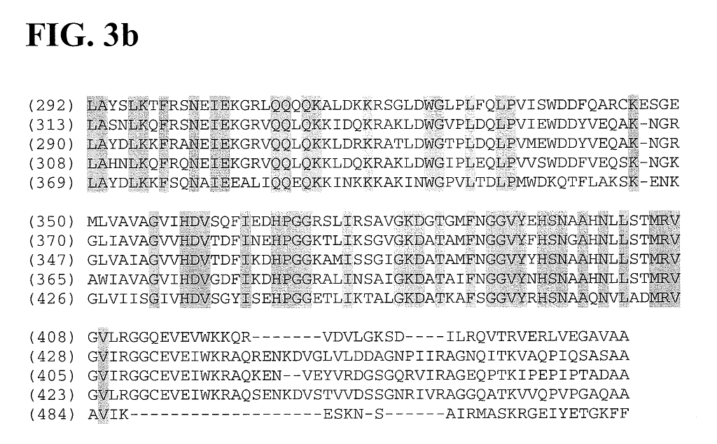

Table 4 and FIG. 3 further illustrate the phylogenetic relationship of newly-identified proteins, LnD9DS-1, LnD9DS-2, and MgD9DS, as well as AnD9DS and the yeast desaturase, ScOLE1. The LnD9DS-1, MgD9DS, and AnD9DS (sdeA) amino acids sequences share a greater percentage of identity with one another as compared to LnD9DS-2. The conservation of amino acid identity allows us to predict that the substrate specificity for 18:0 acyl-CoA is dependent upon the conserved sequence shared between LnD9DS-1, MgD9DS, and AnD9DS (sdeA). In comparison, the acyl-CoA substrate specificity of LnD9DS-2 is preferential for 16:0 as a result of its divergent amino acid sequence.

TABLE 4
Amino acid identity of various fungal desaturase
sequences aligned using ClustalW.

| | LnD9DS-1 | LnD9DS-2 | MgD9DS | Yeast OLE1 |
|---|---|---|---|---|
| AnD9DS (sdeA) | 81% | 61% | 75% | 49% |
| LnD9DS-1 | — | 61% | 75% | 47% |
| LnD9DS-2 | 61% | — | 62% | 49% |
| MgD9DS | 75% | 62% | — | 49% |

### Example 2: Design and Synthesis of Optimized Delta-9 Desaturase Genes from Magnaporthe grisea, Helicoverpa Zea, and Leptosphaeria nodorum

To obtain higher expression of fungal delta-9 desaturase genes in canola, we engineered these genes so that they are more efficiently expressed in transgenic canola cells containing the heterologous gene. Extensive analysis of the DNA sequence of the native Magnaporthe grisea, Helicoverpa zea and Leptosphaeria nodorum delta-9 desaturase coding regions disclosed herein as SEQ ID NO:9, SEQ ID NO:10 and SEQ ID NO:11, respectively, revealed the presence of several sequence motifs that are thought to be detrimental to optimal plant expression, as well as a non-optimal codon composition for such optimal plant expression. In order to design optimized genes encoding a delta-9 desaturase protein, we generated DNA sequences in silico that are more “plant-like” (and specifically, more “canola-like”) in nature, in which the sequence modifications do not hinder translation or create mRNA instability.

To engineer plant-optimized genes encoding a delta-9 desaturase, DNA sequences were designed to encode the amino acid sequences of the protein desaturases, utilizing a redundant genetic code established from a codon bias table compiled from the protein coding sequences for the particular host plants (i.e., canola). Preferred codon usages for canola are shown in Table 5. Columns C and G of Table 5 present the distributions (in % of usage for all codons for that amino acid) of synonymous codons for each amino acid, as found in the coding regions of Brassica napus. It is evident that some synonymous codons for some amino acids are found only rarely in plant genes (e.g., CGG in canola). A codon was considered to be rarely used if it is represented at about 10% or less of the time to encode the relevant amino acid in genes of either plant type (indicated by “DNU” in Columns D and H of Table 5). To balance the distribution of the remaining codon choices for an amino acid, a Weighted Average representation for each codon was calculated, using the formula:

Weighted Average % of C1=1/(% C1+% C2+% C3+etc.)×% C1×100,
where C1 is the codon in question and % C2, % C3, etc. represent the averages of the % values for Brassica napus of remaining synonymous codons (average % values for the relevant codons are taken from Columns C and G) of Table 5.

The Weighted Average % value for each codon is given in Columns D and H of Table 5.

TABLE 5
Synonomous codon representation in coding regions of canola
(B. napus) genes (Columns C and G). Values for a balanced-
biased codon representation set for a plant-optimized
synthetic gene design are in Columns D and H.

| A | | | D | E | | | H |
|---|---|---|---|---|---|---|---|
| Amino | B | C | Weighted | Amino | F | G | Weighted |
| Acid | Codon | Canola % | Average | Acid | Codon | Canola % | Average |
| ALA (A) | GCA | 23.3 | 23.3 | LEU (L) | CTA | 10.1 | DNU |
| | GCC | 21.2 | 21.2 | | CTC | 22.8 | 28.5 |
| | GCG | 14.2 | 14.2 | | CTG | 11.6 | 14.6 |
| | GCT | 41.3 | 41.3 | | CTT | 25.2 | 31.6 |
| ARG (R) | AGA | 31.8 | 43.8 | | TTA | 10.1 | DNU |
| | AGG | 22.1 | 30.5 | | TTG | 20.2 | 25.3 |
| | CGA | 9.9 | DNU | LYS (K) | AAA | 44.6 | 44.6 |
| | CGC | 8.9 | DNU | | AAG | 55.4 | 55.4 |
| | CGG | 8.6 | DNU | MET (M) | ATG | 100.0 | 100.0 |
| | CGT | 18.6 | 25.7 | PHE (F) | TTC | 58.6 | 58.6 |
| ASN (N) | AAC | 62.6 | 62.6 | | TTT | 41.4 | 41.4 |
| | AAT | 37.4 | 37.4 | PRO (P) | CCA | 29.6 | 29.6 |
| ASP (D) | GAC | 42.5 | 42.5 | | CCC | 14.6 | 14.6 |
| | GAT | 57.5 | 57.5 | | CCG | 18.4 | 18.4 |
| CYS (C) | TGC | 49.2 | 49.2 | | CCT | 37.3 | 37.3 |
| | TGT | 50.8 | 50.8 | SER (S) | AGC | 16.0 | 17.9 |
| END | TAA | 38.5 | DNU | | AGT | 14.1 | 15.8 |
| | TAG | 22.1 | DNU | | TCA | 18.2 | 20.4 |
| | TGA | 39.4 | 100.0 | | TCC | 16.7 | 18.7 |
| GLN (Q) | CAA | 50.0 | 50.0 | | TCG | 10.7 | DNU |
| | CAG | 50.0 | 50.0 | | TCT | 24.3 | 27.2 |
| GLU (E) | GAA | 43.6 | 43.6 | THR (T) | ACA | 26.3 | 26.3 |
| | GAG | 56.4 | 56.4 | | ACC | 26.9 | 26.9 |
| GLY (G) | GGA | 36.4 | 36.4 | | ACG | 16.9 | 16.9 |
| | GGC | 16.2 | 16.2 | | ACT | 30.0 | 30.0 |
| | GGG | 15.2 | 15.2 | TRP (W) | TGG | 100.0 | 100.0 |
| | GGT | 32.1 | 32.1 | TYR (Y) | TAC | 59.4 | 59.4 |
| HIS (H) | CAC | 49.6 | 49.6 | | TAT | 40.6 | 40.6 |
| | CAT | 50.4 | 50.4 | VAL (V) | GTA | 10.8 | DNU |
| ILE (I) | ATA | 21.1 | 21.1 | | GTC | 24.1 | 27.0 |
| | ATC | 42.7 | 42.7 | | GTG | 28.3 | 31.7 |
| | ATT | 36.2 | 36.2 | | GTT | 36.8 | 41.3 |

**NA = Not Applicable
***DNU = Do Not Use

New DNA sequences which encode essentially the amino acid sequence of the Magnaporthe grisea, Helicoverpa zea, and Leptosphaeria nodorum delta-9 desaturases of SEQ ID NO:12, SEQ ID NO:13 and SEQ ID NO:14, respectively, were designed for optimal expression in canola using a first and second choice codon distribution of frequently used codons found in canola genes. The new DNA sequences differ from the native DNA sequences encoding the delta-9 desaturase proteins by the substitution of plant-preferred (i.e., first preferred, second preferred, third preferred, or fourth preferred) codons to specify an appropriate amino acid at each position within the protein amino acid sequence.

Design of the plant-optimized DNA sequences were initiated by reverse-translation of the protein sequences of SEQ ID NO:12, SEQ ID NO:13 and SEQ ID NO:14, using a canola codon bias table constructed from Table 5 Columns D and H. The initial sequences were then modified by compensating codon changes (while retaining overall weighted average codon representation) to remove restriction enzyme recognition sites, remove highly stable intrastrand secondary structures, and remove other sequences that might be detrimental to cloning manipulations or expression of the engineered gene in plants. The DNA sequences were then re-analyzed for restriction enzyme recognition sites that might have been created by the modifications. The identified sites were then further modified by replacing the relevant codons with first, second, third, or fourth choice preferred codons. The modified sequences were further analyzed and further modified to reduce the frequency of TA and CG doublets, and to increase the frequency of TG and CT doublets. In addition to these doublets, sequence blocks that have more than about six consecutive residues of [G+C] or [A+T] were modified by replacing the codons of first or second choice, etc. with other preferred codons of choice. Rarely used codons were not included to a substantial extent in the gene design, and were used only when necessary to accommodate a different design criterion than codon composition per se (e.g., addition or deletion of restriction enzyme recognition sites). Exemplary synthetic canola-optimized desaturase DNA sequences designed by this process are listed in SEQ ID NO:15, SEQ ID NO:16, and SEQ ID NO:17.

The resulting DNA sequences, as represented by SEQ ID NOs:15-17, have a higher degree of codon diversity and a desirable base composition. Furthermore, these sequences contain strategically placed restriction enzyme recognition sites, and lack sequences that might interfere with transcription of the gene, or translation of the product mRNA. Tables 6-8 present a comparison of the codon compositions of the coding regions for the delta-9 desaturase proteins as found in the native gene, and in the plant-optimized versions, and compare both to the codon composition recommendations for a plant-optimized sequence as calculated from Table 5 Columns D and H.

TABLE 6
Codon compositions of coding regions for a MgD9DS protein. The native M. grisa
desaturase coding region is compared to a Plant-Optimized version.

| | | | | | | | | | | | | | Pint |
|---|---|---|---|---|---|---|---|---|---|---|---|---|---|
| Amino | | Native | Native | Pint Opt | Pint Opt | Pint Opt | Amino | | Native | Native | Pint Opt | Pint Opt | Opt |
| Acid | Codon | Gene # | Gene % | Gene # | Gene % | Recm'd | Acid | Codon | Gene # | Gene % | Gene # | Gene % | Recm'd |
| ALA (A) | GCA | 4 | 10.5 | 10 | 26.3 | 23.3 | LEU (L) | CTA | 0 | 0.0 | 0 | 0.0 | 0.0 |
| | GCC | 18 | 47.4 | 8 | 21.1 | 21.2 | | CTC | 11 | 28.9 | 11 | 28.9 | 28.5 |
| | GCG | 3 | 7.9 | 4 | 10.5 | 14.2 | | CTG | 11 | 28.9 | 5 | 13.2 | 14.6 |
| | GCT | 13 | 34.2 | 16 | 42.1 | 41.3 | | CTT | 12 | 31.6 | 12 | 31.6 | 31.6 |
| ARG (R) | AGA | 2 | 9.5 | 10 | 47.6 | 43.8 | | TTA | 0 | 0.0 | 0 | 0.0 | 0.0 |
| | AGG | 1 | 4.8 | 6 | 28.6 | 30.5 | | TTG | 4 | 10.5 | 10 | 26.3 | 25.3 |
| | CGA | 2 | 9.5 | 0 | 0.0 | 0.0 | LYS (K) | AAA | 1 | 3.4 | 13 | 44.8 | 44.6 |
| | CGC | 12 | 57.1 | 0 | 0.0 | 0.0 | | AAG | 28 | 96.6 | 16 | 55.2 | 55.4 |
| | CGG | 0 | 0.0 | 0 | 0.0 | 0.0 | MET (M) | ATG | 7 | 100 | 7 | 100 | 100.0 |
| | CGT | 4 | 19.0 | 5 | 23.8 | 25.7 | PHE (F) | TTC | 17 | 89.5 | 11 | 57.9 | 58.6 |
| ASN (N) | AAC | 23 | 100.0 | 14 | 60.9 | 62.6 | | TTT | 2 | 10.5 | 8 | 42.1 | 41.4 |
| | AAT | 0 | 0.0 | 9 | 39.1 | 37.4 | PRO (P) | CCA | 0 | 0.0 | 6 | 28.6 | 29.6 |
| ASP (D) | GAC | 17 | 68.0 | 11 | 44.0 | 42.5 | | CCC | 9 | 42.9 | 3 | 14.3 | 14.6 |
| | GAT | 8 | 32.0 | 14 | 56.0 | 57.5 | | CCG | 5 | 23.8 | 4 | 19.0 | 18.4 |
| CYS (C) | TGC | 2 | 66.7 | 1 | 33.3 | 49.2 | | CCT | 7 | 33.3 | 8 | 38.1 | 37.3 |
| | TGT | 1 | 33.3 | 2 | 66.7 | 50.8 | SER (S) | AGC | 3 | 10.7 | 5 | 17.9 | 17.9 |
| END | TAA | 0 | 0.0 | 0 | 0.0 | 0.0 | | AGT | 0 | 0.0 | 4 | 14.3 | 15.8 |
| | TAG | 0 | 0.0 | 0 | 0.0 | 0.0 | | TCA | 5 | 17.9 | 7 | 25.0 | 20.4 |
| | TGA | 1 | 100.0 | 1 | 100.0 | 100.0 | | TCC | 9 | 32.1 | 4 | 14.3 | 18.7 |
| GLN (Q) | CAA | 2 | 9.5 | 11 | 52.4 | 50.0 | | TCG | 9 | 32.1 | 0 | 0.0 | 0.0 |
| | CAG | 19 | 90.5 | 10 | 47.6 | 50.0 | | TCT | 2 | 7.1 | 8 | 28.6 | 27.2 |
| GLU (E) | GAA | 1 | 6.7 | 7 | 46.7 | 43.6 | THR (T) | ACA | 4 | 16.7 | 6 | 25.0 | 26.3 |
| 16 | GAG | 14 | 93.3 | 8 | 53.3 | 56.4 | | ACC | 15 | 62.5 | 7 | 29.2 | 26.9 |
| GLY (G) | GGA | 8 | 19.5 | 15 | 36.6 | 36.4 | | ACG | 1 | 4.2 | 4 | 16.7 | 16.9 |
| | GGC | 13 | 31.7 | 7 | 17.1 | 16.2 | | ACT | 4 | 16.7 | 7 | 29.2 | 30.0 |
| | GGG | 1 | 2.4 | 6 | 14.6 | 15.2 | TRP (W) | TGG | 21 | 100 | 21 | 100 | 100.0 |
| | GGT | 19 | 46.3 | 13 | 31.7 | 32.1 | TYR (Y) | TAC | 16 | 94.1 | 10 | 58.8 | 59.4 |
| HIS (H) | CAC | 19 | 95.0 | 10 | 50.0 | 49.6 | | TAT | 1 | 5.9 | 7 | 41.2 | 40.6 |
| | CAT | 1 | 5.0 | 10 | 50.0 | 50.4 | VAL (V) | GTA | 1 | 2.5 | 0 | 0.0 | 0.0 |
| ILE (I) | ATA | 1 | 4.2 | 5 | 20.8 | 21.1 | | GTC | 21 | 52.5 | 11 | 27.5 | 27.0 |
| | ATC | 15 | 62.5 | 10 | 41.7 | 42.7 | | GTG | 4 | 10.0 | 13 | 32.5 | 31.7 |
| | ATT | 8 | 33.3 | 9 | 37.5 | 36.2 | | GTT | 14 | 35.0 | 16 | 40.0 | 41.3 |
| | Totals | 232 | | 232 | | | | Totals | 244 | | 244 | | |

TABLE 7
Codon compositions of coding regions for a HzD9DS protein. The native H. zea
desaturase coding region is compared to a Plant-Optimized version.

| | | | | | | | | | | | | | Pint |
|---|---|---|---|---|---|---|---|---|---|---|---|---|---|
| Amino | | Native | Native | Pint Opt | Pint Opt | Pint Opt | Amino | | Native | Native | Pint Opt | Pint Opt | Opt |
| Acid | Codon | Gene # | Gene % | Gene # | Gene % | Recm'd | Acid | Codon | Gene # | Gene % | Gene # | Gene % | Recm'd |
| ALA (A) | GCA | 4 | 11.4 | 9 | 25.7 | 23.3 | LEU (L) | CTA | 2 | 5.9 | 0 | 0.0 | 0.0 |
| | GCC | 7 | 20.0 | 7 | 20.0 | 21.2 | | CTC | 8 | 23.5 | 10 | 29.4 | 28.5 |
| | GCG | 8 | 22.9 | 4 | 11.4 | 14.2 | | CTG | 14 | 41.2 | 6 | 17.6 | 14.6 |
| | GCT | 16 | 45.7 | 15 | 42.9 | 41.3 | | CTT | 6 | 17.6 | 10 | 29.4 | 31.6 |
| ARG (R) | AGA | 1 | 7.7 | 6 | 46.2 | 43.8 | | TTA | 2 | 5.9 | 0 | 0.0 | 0.0 |
| | AGG | 5 | 38.5 | 4 | 30.8 | 30.5 | | TTG | 2 | 5.9 | 8 | 23.5 | 25.3 |
| | CGA | 2 | 15.4 | 0 | 0.0 | 0.0 | LYS (K) | AAA | 11 | 44.0 | 10 | 40.0 | 44.6 |
| | CGC | 5 | 38.5 | 0 | 0.0 | 0.0 | | AAG | 14 | 56.0 | 15 | 60.0 | 55.4 |
| | CGG | 0 | 0.0 | 0 | 0.0 | 0.0 | MET (M) | ATG | 8 | 100 | 8 | 100 | 100.0 |
| | CGT | 0 | 0.0 | 3 | 23.1 | 25.7 | PHE (F) | TTC | 20 | 83.3 | 14 | 58.3 | 58.6 |
| ASN (N) | AAC | 13 | 72.2 | 11 | 61.1 | 62.6 | | TTT | 4 | 16.7 | 10 | 41.7 | 41.4 |
| | AAT | 5 | 27.8 | 7 | 38.9 | 37.4 | PRO (P) | CCA | 1 | 6.3 | 5 | 31.3 | 29.6 |
| ASP (D) | GAC | 16 | 64.0 | 12 | 48.0 | 42.5 | | CCC | 5 | 31.3 | 3 | 18.8 | 14.6 |
| | GAT | 9 | 36.0 | 13 | 52.0 | 57.5 | | CCG | 2 | 12.5 | 2 | 12.5 | 18.4 |
| CYS (C) | TGC | 1 | 100.0 | 0 | 0.0 | 49.2 | | CCT | 8 | 50.0 | 6 | 37.5 | 37.3 |
| | TGT | 0 | 0.0 | 1 | 100.0 | 50.8 | SER (S) | AGC | 2 | 12.5 | 3 | 18.8 | 17.9 |
| END | TAA | 1 | 100.0 | 0 | 0.0 | 0.0 | | AGT | 1 | 6.3 | 3 | 18.8 | 15.8 |
| | TAG | 0 | 0.0 | 0 | 0.0 | 0.0 | | TCA | 1 | 6.3 | 3 | 18.8 | 20.4 |
| | TGA | 0 | 0.0 | 1 | 100.0 | 100.0 | | TCC | 6 | 37.5 | 3 | 18.8 | 18.7 |
| GLN (Q) | CAA | 2 | 33.3 | 3 | 50.0 | 50.0 | | TCG | 3 | 18.8 | 0 | 0.0 | 0.0 |
| | CAG | 4 | 66.7 | 3 | 50.0 | 50.0 | | TCT | 3 | 18.8 | 4 | 25.0 | 27.2 |
| GLU (E) | GAA | 7 | 63.6 | 5 | 45.5 | 43.6 | THR (T) | ACA | 3 | 16.7 | 5 | 27.8 | 26.3 |
| 16 | GAG | 4 | 36.4 | 6 | 54.5 | 56.4 | | ACC | 7 | 38.9 | 5 | 27.8 | 26.9 |
| GLY (G) | GGA | 8 | 40.0 | 9 | 45.0 | 36.4 | | ACG | 4 | 22.2 | 3 | 16.7 | 16.9 |
| | GGC | 6 | 30.0 | 4 | 20.0 | 16.2 | | ACT | 4 | 22.2 | 5 | 27.8 | 30.0 |
| | GGG | 2 | 10.0 | 3 | 15.0 | 15.2 | TRP (W) | TGG | 14 | 100 | 14 | 100 | 100.0 |
| | GGT | 4 | 20.0 | 4 | 20.0 | 32.1 | TYR (Y) | TAC | 12 | 80.0 | 9 | 60.0 | 59.4 |
| HIS (H) | CAC | 11 | 73.3 | 8 | 53.3 | 49.6 | | TAT | 3 | 20.0 | 6 | 40.0 | 40.6 |
| | CAT | 4 | 26.7 | 7 | 46.7 | 50.4 | VAL (V) | GTA | 0 | 0.0 | 0 | 0.0 | 0.0 |
| ILE (I) | ATA | 3 | 15.0 | 4 | 20.0 | 21.1 | | GTC | 5 | 26.3 | 5 | 26.3 | 27.0 |
| | ATC | 10 | 50.0 | 9 | 45.0 | 42.7 | | GTG | 13 | 68.4 | 6 | 31.6 | 31.7 |
| | ATT | 7 | 35.0 | 7 | 35.0 | 36.2 | | GTT | 1 | 5.3 | 8 | 42.1 | 41.3 |
| | Totals | 165 | | 165 | | | | Totals | 189 | | 189 | | |

TABLE 8
Codon compositions of coding regions for a LnD9DS-2 protein. The native L. nodorum
desaturase coding region is compared to a Plant-Optimized version.

| | | | | | | | | | | | | | Pint |
|---|---|---|---|---|---|---|---|---|---|---|---|---|---|
| Amino | | Native | Native | Pint Opt | Pint Opt | Pint Opt | Amino | | Native | Native | Pint Opt | Pint Opt | Opt |
| Acid | Codon | Gene # | Gene % | Gene # | Gene % | Recm'd | Acid | Codon | Gene # | Gene % | Gene # | Gene % | Recm'd |
| ALA (A) | GCA | 3 | 9.4 | 7 | 21.9 | 23.3 | LEU (L) | CTA | 7 | 15.6 | 0 | 0.0 | 0.0 |
| | GCC | 9 | 28.1 | 7 | 21.9 | 21.2 | | CTC | 14 | 31.1 | 13 | 28.9 | 28.5 |
| | GCG | 12 | 37.5 | 5 | 15.6 | 14.2 | | CTG | 7 | 15.6 | 7 | 15.6 | 14.6 |
| | GCT | 8 | 25.0 | 13 | 40.6 | 41.3 | | CTT | 5 | 11.1 | 14 | 31.1 | 31.6 |
| ARG (R) | AGA | 4 | 13.8 | 13 | 44.8 | 43.8 | | TTA | 3 | 6.7 | 0 | 0.0 | 0.0 |
| | AGG | 3 | 10.3 | 9 | 31.0 | 30.5 | | TTG | 9 | 20.0 | 11 | 24.4 | 25.3 |
| | CGA | 7 | 24.1 | 0 | 0.0 | 0.0 | LYS (K) | AAA | 9 | 45.0 | 9 | 45.0 | 44.6 |
| | CGC | 8 | 27.6 | 0 | 0.0 | 0.0 | | AAG | 11 | 55.0 | 11 | 55.0 | 55.4 |
| | CGG | 5 | 17.2 | 0 | 0.0 | 0.0 | MET (M) | ATG | 9 | 100 | 9 | 100 | 100.0 |
| | CGT | 2 | 6.9 | 7 | 24.1 | 25.7 | PHE (F) | TTC | 16 | 80.0 | 12 | 60.0 | 58.6 |
| ASN (N) | AAC | 6 | 50.0 | 8 | 66.7 | 62.6 | | TTT | 4 | 20.0 | 8 | 40.0 | 41.4 |
| | AAT | 6 | 50.0 | 4 | 33.3 | 37.4 | PRO (P) | CCA | 3 | 16.7 | 5 | 27.8 | 29.6 |
| ASP (D) | GAC | 16 | 66.7 | 10 | 41.7 | 42.5 | | CCC | 8 | 44.4 | 3 | 16.7 | 14.6 |
| | GAT | 8 | 33.3 | 14 | 58.3 | 57.5 | | CCG | 2 | 11.1 | 3 | 16.7 | 18.4 |
| CYS (C) | TGC | 4 | 80.0 | 2 | 40.0 | 49.2 | | CCT | 5 | 27.8 | 7 | 38.9 | 37.3 |
| | TGT | 1 | 20.0 | 3 | 60.0 | 50.8 | SER (S) | AGC | 8 | 27.6 | 5 | 17.2 | 17.9 |
| END | TAA | 0 | 0.0 | 0 | 0.0 | 0.0 | | AGT | 6 | 20.7 | 5 | 17.2 | 15.8 |
| | TAG | 1 | 100.0 | 0 | 0.0 | 0.0 | | TCA | 1 | 3.4 | 6 | 20.7 | 20.4 |
| | TGA | 0 | 0.0 | 1 | 100.0 | 100.0 | | TCC | 6 | 20.7 | 5 | 17.2 | 18.7 |
| GLN (Q) | CAA | 10 | 55.6 | 10 | 55.6 | 50.0 | | TCG | 7 | 24.1 | 0 | 0.0 | 0.0 |
| | CAG | 8 | 44.4 | 8 | 44.4 | 50.0 | | TCT | 1 | 3.4 | 8 | 27.6 | 27.2 |
| GLU (E) | GAA | 5 | 33.3 | 7 | 46.7 | 43.6 | THR (T) | ACA | 11 | 44.0 | 7 | 28.0 | 26.3 |
| 16 | GAG | 10 | 66.7 | 8 | 53.3 | 56.4 | | ACC | 5 | 20.0 | 7 | 28.0 | 26.9 |
| GLY (G) | GGA | 13 | 34.2 | 14 | 36.8 | 36.4 | | ACG | 7 | 28.0 | 4 | 16.0 | 16.9 |
| | GGC | 16 | 42.1 | 6 | 15.8 | 16.2 | | ACT | 2 | 8.0 | 7 | 28.0 | 30.0 |
| | GGG | 6 | 15.8 | 6 | 15.8 | 15.2 | TRP (W) | TGG | 19 | 100 | 19 | 100 | 100.0 |
| | GGT | 3 | 7.9 | 12 | 31.6 | 32.1 | TYR (Y) | TAC | 11 | 64.7 | 10 | 58.8 | 59.4 |
| HIS (H) | CAC | 12 | 66.7 | 9 | 50.0 | 49.6 | | TAT | 6 | 35.3 | 7 | 41.2 | 40.6 |
| | CAT | 6 | 33.3 | 9 | 50.0 | 50.4 | VAL (V) | GTA | 6 | 17.6 | 0 | 0.0 | 0.0 |
| ILE (I) | ATA | 4 | 18.2 | 5 | 22.7 | 21.1 | | GTC | 10 | 29.4 | 9 | 26.5 | 27.0 |
| | ATC | 9 | 40.9 | 10 | 45.5 | 42.7 | | GTG | 12 | 35.3 | 11 | 32.4 | 31.7 |
| | ATT | 9 | 40.9 | 7 | 31.8 | 36.2 | | GTT | 6 | 17.6 | 14 | 41.2 | 41.3 |
| | Totals | 214 | | 214 | | | | Totals | 236 | | 236 | | |

Syntheses of DNA fragments comprising SEQ ID NO:15, SEQ ID NO:16, and SEQ ID NO:17 were performed by commercial suppliers (PicoScript, Houston, Tex. and Blue Heron Biotechnology, Bothell, Wash.). These canola-optimized sequences were labeled as version 2 (v2). The synthetic DNA fragments were then cloned into expression vectors, and transformed into Agrobacterium and canola as described in the Examples below.

### Example 3: Plasmid Construction

The following plasmids were constructed using standard molecular biology techniques. Polynucleotide fragments containing plant transcription units (comprised of a promoter linked to a gene of interest, terminated by a 3′UTR), or “PTUs,” were constructed and combined with additional plant transcription units within the T-strand region of a binary vector.

Description of pDAB7318:

pDAB7318 (FIG. 6; SEQ ID NO:58) was constructed using standard molecular biology techniques. This plasmid contains two desaturase PTU sequences. The first desaturase PTU contains the Phaseolus vulgaris phaseolin promoter (PvPhas promoter v2 (SEQ ID NO:67); Genbank: J01263), Phaseolus vulgaris 5′ untranslated region (PvPhas 5′ UTR (SEQ ID NO:68); Genbank: J01263), AnD9DS v3 gene (SEQ ID NO:49), Phaseolus vulgaris 3′ untranslated region (PvPhas 3′ UTR v1 (SEQ ID NO:69); Genbank: J01263) and Phaseolus vulgaris matrix attachment region (PvPhas 3′ MAR v2 (SEQ ID NO:70); Genbank: J01263). The second desaturase PTU contains the PvPhas promoter v2, PvPhas 5′ UTR, LnD9DS-2 v2 (SEQ ID NO:17), and Agrobacterium tumefaciens ORF23 3′ untranslated region (AtuORF23 3′ UTR (SEQ ID NO:71); Huang et al. (1990) J. Bacteriol. 172:1814-22).

The elements in the desaturase PTUs are connected by additional short intervening sequences. The two desaturase PTU sequences are flanked by Invitrogen's Gateway® Recombination sites, which are used to facilitate the transfer of these PTU expression cassettes into the Agrobacterium transformation plasmid. Additionally, the plasmid contains an origin of replication, and a kanamycin selectable marker.

Description of pDAB7319:

pDAB7319 (FIG. 7; SEQ ID NO:60) was constructed via Gateway® recombination between pDAB7318 and pDAB7309 (FIG. 5; SEQ ID NO:53). This plasmid contains the two desaturase PTU sequences set forth in the preceding “Description of pDAB7318.” These PTUs were orientated in a head-to-tail orientation within the T-strand DNA border regions of the plant transformation binary vector, pDAB7309. This binary vector contains the phosphinothricin acetyl transferase PTU, which consists of the Cassava vein Mosaic Virus Promoter (CsVMV promoter v2; Verdaguer et al. (1996) Plant Mol. Biol. 31:1129-39); phosphinothricin acetyl transferase (PAT v5; Wohlleben et al. (1988) Gene 70:25-37); and Agrobacterium tumefaciens ORF1 3′ untranslated region (AtuORF1 3′UTR v4; Huang et al. (1990), supra), in addition to other regulatory elements such as the Nicotiana tabacum RB7 Matrix Attachment Region (RB7 MARv2; Genbank: U67919), Overdrive (Toro et al. (1988) Proc. Natl. Acad. Sci. U.S.A. 85(22):8558-62), and T-strand border sequences (T-DNA Border A and T-DNA Border B; Gardner et al. (1986) Science 231:725-7, and PCT International Patent Publication No. WO2001/025459A1). Plasmids containing the PTUs described above were isolated and confirmed via restriction enzyme digestion and DNA sequencing.

Description of pDAB7320:

pDAB7320 (FIG. 8; SEQ ID NO:55) was constructed using standard molecular biology techniques. This plasmid contains one desaturase PTU sequence. The desaturase PTU contains the PvPhas promoter v2, PvPhas 5′ UTR, LnD9DS-2 v2 (SEQ ID NO:17), and the AtuORF23 3′ UTR. The elements in the desaturase PTUs are connected by additional short intervening sequences. The desaturase PTU sequence also is flanked by Invitrogen's Gateway® Recombination sites to facilitate its transfer into an Agrobacterium transformation plasmid. Additionally, the plasmid contains an origin of replication and kanamycin selectable marker.

Description of pDAB7321:

pDAB7321 (FIG. 9; SEQ ID NO:61) was constructed via Gateway® recombination between pDAB7320 and pDAB7309. This plasmid contains the desaturase PTU sequence set forth in the preceding “Description of pDAB7319.” This PTU was orientated in a head-to-tail orientation within the T-strand DNA border regions of the plant transformation binary vector, pDAB7309. This binary vector contains the phosphinothricin acetyl transferase PTU: CsVMV promoter v2; PAT v5; and AtuORF1 3′UTR v4, in addition to other regulatory elements such as Overdrive and T-strand border sequences (T-DNA Border A and T-DNA Border B). Plasmids containing the PTU described above were isolated and confirmed via restriction enzyme digestion and DNA sequencing.

Description of pDAB7323:

pDAB7323 (FIG. 10; SEQ ID NO:56) was constructed using standard molecular biology techniques. This plasmid contains two desaturase PTU sequences. The first desaturase PTU contains the PvPhas promoter v2, PvPhas 5′ UTR, AnD9DS v3 (SEQ ID NO:47), PvPhas 3′ UTR, and PvPhas 3′ MAR v2. The second desaturase PTU contains the PvPhas promoter v2, PvPhas 5′ UTR, HzD9DS v2 (SEQ ID NO:16), and AtuORF23 3′ UTR. The elements in the desaturase PTUs are connected by additional short intervening sequences. The two desaturase PTU sequences are flanked by Invitrogen's Gateway® Recombination sites to facilitate their transfer into an Agrobacterium transformation plasmid. Additionally, the plasmid contains an origin of replication and kanamycin selectable marker.

Description of pDAB7324:

pDAB7324 (FIG. 11; SEQ ID NO:62) was constructed via Gateway® recombination between pDAB7323 and pDAB7309. This plasmid contains the two desaturase PTU sequences set forth in the preceding “Description of pDAB7323.” These PTUs were orientated in a head-to-tail orientation within the T-strand DNA border regions of the plant transformation binary vector, pDAB7309. This binary vector contains the phosphinothricin acetyl transferase PTU: CsVMV promoter v2; PAT v5; and AtuORF1 3′UTR v4, in addition to other regulatory elements such as Overdrive and T-stand border sequences (T-DNA Border A and T-DNA Border B). Plasmids containing the PTUs described above were isolated and confirmed via restriction enzyme digestion and DNA sequencing.

Description of pDAB7325:

pDAB7325 (FIG. 12; SEQ ID NO:57) was constructed using standard molecular biology techniques. This plasmid contains one desaturase PTU sequence. This desaturase PTU contains the PvPhas promoter v2, PvPhas 5′ UTR, HzD9DS v2 (SEQ ID NO:16), and AtuORF23 3′ UTR. The elements in the desaturase PTU are connected by additional short intervening sequences, and the desaturase PTU sequence is flanked by Invitrogen's Gateway® Recombination sites to facilitate its transfer into an Agrobacterium transformation plasmid. Additionally, the plasmid contains an origin of replication and kanamycin selectable marker.

Description of pDAB7326:

pDAB7326 (FIG. 13; SEQ ID NO:63) was constructed via Gateway® recombination between pDAB7325 and pDAB7309. This plasmid contains the desaturase PTU sequence set forth in the preceding “Description of pDAB7325.” The PTU was orientated in a head-to-tail orientation within the T-strand DNA border regions of the plant transformation binary vector, pDAB7309. This binary vector contains the phosphinothricin acetyl transferase PTU: CsVMV promoter v2; PAT v5; and AtuORF1 3′UTR v4, in addition to other regulatory elements such as Overdrive and T-stand border sequences (T-DNA Border A and T-DNA Border B). Plasmids containing the PTU described above were isolated and confirmed via restriction enzyme digestion and DNA sequencing.

Description of pDAB7327:

pDAB7327 (FIG. 14; SEQ ID NO:58) was constructed using standard molecular biology techniques. This plasmid contains one desaturase PTU sequence. The desaturase PTU contains the PvPhas promoter v2, PvPhas 5′ UTR, AnD9DS v3 gene (SEQ ID NO:49), and AtuORF23 3′ UTR. The elements in the desaturase PTU are connected by additional short intervening sequences. The desaturase PTU sequence also is flanked by Invitrogen's Gateway® Recombination sites to facilitate its transfer into an Agrobacterium transformation plasmid. Additionally, the plasmid contains an origin of replication and kanamycin selectable marker.

Description of pDAB7328:

pDAB7328 (FIG. 15; SEQ ID NO:64) was constructed via Gateway® recombination between pDAB7327 and pDAB7309. This plasmid contains the desaturase PTU sequence set forth in the preceding “Description of pDAB7327.” This PTU was orientated in a head-to-tail orientation within the T-strand DNA border regions of the plant transformation binary vector, pDAB7309. This binary vector contains the phosphinothricin acetyl transferase PTU: CsVMV promoter v2; PAT v5; and AtuORF1 3′UTR v4, in addition to other regulatory elements such as Overdrive and T-stand border sequences (T-DNA Border A and T-DNA Border B). Plasmids containing the PTU described above were isolated and confirmed via restriction enzyme digestion and DNA sequencing.

Description of pDAB7329:

pDAB7329 (FIG. 16; SEQ ID NO:59) was constructed using standard molecular biology techniques. This plasmid contains one desaturase PTU sequence, which contains the PvPhas promoter v2, PvPhas 5′ UTR, MgD9DS v2 (SEQ ID NO:15), and AtuORF23 3′ UTR. The elements in this desaturase PTU are connected by additional short intervening sequences. The desaturase PTU sequence is flanked by Invitrogen's Gateway® Recombination sites to facilitate its transfer into an Agrobacterium transformation plasmid. Additionally, the plasmid contains an origin of replication and kanamycin selectable marker.

Description of pDAB7330:

pDAB7330 (FIG. 17; SEQ ID NO:65) was constructed via Gateway® recombination between pDAB7329 and pDAB7309. This plasmid contains the desaturase PTU sequence set forth in the preceding “Description of pDAB7325.” This PTU was orientated in a head-to-tail orientation within the T-strand DNA border regions of the plant transformation binary vector, pDAB7309. This binary vector contains the phosphinothricin acetyl transferase PTU: CsVMV promoter v2; PAT v5; and AtuORF1 3′UTR v4, in addition to other regulatory elements such as Overdrive and T-stand border sequences (T-DNA Border A and T-DNA Border B). Plasmids containing the PTU described above were isolated and confirmed via restriction enzyme digestion and DNA sequencing.

Description of pDAB73311:

In addition to the foregoing, a control plasmid that did not contain a desaturase PTU was constructed (SEQ ID NO:66). FIG. 18. This construct only contained the phosphinothricin acetyl transferase PTU, in addition to the other regulatory elements described in pDAB7309.

### Example 4: Agrobacterium Transformation

Electro-competent Agrobacterium tumefaciens cells (Table 9) were prepared using a protocol from Weigel and Glazebrook (2002) “How to Transform Arabidopsis,” Ch. 5, in Arabidopsis, Cold Spring Harbor Laboratory Press, Cold Spring Harbor, N.Y. 50 μL of competent Agrobacterium cells were thawed on ice, and were transformed using 300 to 400 ng of binary vector plasmid DNA. The cell mix was electroporated in the presence of the DNA, using pre-chilled electroporation cuvettes (0.2 cm), and a Bio-Rad Gene Pulser® electroporator (Hercules, Calif.) under the following conditions: Voltage: 2.5 kV, Pulse length: 5 msec, capacitance output 25 μF, resistance 200Ω. After electroporation, 1 mL of YEP broth (Yeast Extract (10 g/L), Peptone (10 g/L), and NaCl (5 g/L)) was added to each cuvette, and the cell-YEP suspension was transferred to a 15 mL culture tube. The cells were incubated at 28° C. with gentle agitation for 4 hours, after which the culture was plated on YEP+agar with the appropriate selection according to Table 9. The plates were incubated for 2-4 days at 28° C., and colonies were selected and streaked onto fresh YEP+agar plates with antibiotic selection and incubated at 28° C. for 1-3 days. Colonies were verified as Agrobacterium using the Ketolactose test, and Ketolactose positive colonies were further isolated using two passages of single colony isolation. A final patch plate was made of colonies after single colony isolation was completed.

TABLE 9
Agrobacterium strains and antibiotic selection.

| | Genomic | Ti Helper | Binary Vector |
|---|---|---|---|
| Strain | Selection | Selection | Selection |
| Z707S | Streptomycin | Kanamycin | Spectinomycin |
| DA2569 | Erythromycin | Kanamycin | Spectinomycin |
| EHA105 | Streptomycin | None Available | Spectinomycin |
| DA2552 | Erythromycin | None | Spectinomycin |

Agrobacterium Colony Validation:

Restriction digestion analysis was used to verify the presence of the intact plasmid by using vector specific restriction digest enzymes. Macherey-Nagel NucleoBond® Plasmid DNA kits were used according to the manufacturer's recommended protocol to purify the plasmid DNA from selected transformed Agrobacterium colonies. Plasmid DNA from the binary vector used in the Agrobacterium transformation was included as a control. Four separate digest reactions were run using 0.75-1 μg of DNA. The reaction was allowed to run for 1-2 hrs, and was then analyzed by agarose gel electrophoresis and ethidium bromide staining. Colonies were selected for which the digests for all enzymes were identical to the plasmid control and matched the expected band sizes.

A. tumefaciens strain LBA404 (Invitrogen Carlsbad, Calif.) was used for Arabidopsis transformation, and A. tumefaciens strain Z707S (Hepburn et al. (1985) J. Gen. Microbiol. 131:2961-9) was used for canola transformation.

### Example 5: Agrobacterium-Mediated Transformation of Arabidopsis thaliana

Arabidopsis Transformation:

Arabidopsis was transformed using a floral dip method based on the method of Clough and Bent (1998) Plant J. 16:735-743. A selected Agrobacterium colony was used to inoculate one or more 30 mL pre-cultures of YEP broth containing appropriate antibiotics for selection. The culture(s) were incubated overnight at 28° C. with constant agitation at 220 rpm. Each pre-culture was used to inoculate two 500 mL cultures of YEP broth containing antibiotics for selection, and the cultures were incubated overnight at 28° C. with constant agitation. The cells were then plated at approx. 8700 g for 10 minutes at room temperature, and the resulting supernatant was discarded. The cell pellet was gently resuspended in 500 mL infiltration media containing: ½× Murashige and Skoog salts/Gamborg's B5 vitamins, 10% (w/v) sucrose, 0.044 μM benzylamino purine (10 μL/liter of 1 mg/mL stock in DMSO), and 300 μL/liter Silwet® L-77. Plants approximately 1 month old were dipped into the media for 15 seconds, with care taken to submerge the newest inflorescence. The plants were then laid down on their sides, and covered (transparent or opaque) for 24 hours, then washed with water, and placed upright. The plants were grown at 22° C., with a 16-hour light/8-hour dark photoperiod. Approximately 4 weeks after dipping, seeds were harvested from the plants.

Arabidopsis thaliana Growth Conditions:

Freshly harvested seed was dried for 7 days at room temperature in the presence of a desiccant. After drying, seed was suspended in a 0.1% Agarose (Sigma Chemical Co., St. Louis, Mo.) solution. The suspended seed was stored at 4° C. for 2 days to complete dormancy requirements and ensure synchronous seed germination (stratification). Sunshine Mix LP5 (Sun Gro Horticulture Inc., Bellevue, Wash.) was covered with fine vermiculite and sub-irrigated with Hoaglan's solution until wet. The soil mix was drained for 24 hours. Stratified seed was sown onto the vermiculite and covered with humidity domes (KORD Products, Bramalea, Ontario, Canada) for 7 days. Seeds were germinated, and plants were grown in a Conviron controller (models CMP4030 and CMP3244, Controlled Environments Limited, Winnipeg, Manitoba, Canada) under long day conditions (16-hours light/8-hours dark) at a light intensity of 120-150 μmol/m² sec under constant temperature (22° C.) and humidity (40-50%). Plants were initially watered with Hoaglan's solution, and subsequently with deionized water to keep the soil moist but not wet. Plants nearing seed harvest (1-2 weeks before harvest) were dried out.

Selection of T₁ Transformed Plants:

T₁ seed was sown on 10.5″×21″ germination trays (T.O. Plastics Inc., Clearwater, Minn.) as described above and grown under the conditions outlined. The domes were removed 5-6 days post-sowing. 5 days post-sowing, and again 10 days post-sowing, seedlings were sprayed with a 0.20% solution of glufosinate herbicide (Liberty) in a spray volume of 10 mL/tray (703 L/ha) using a DeVilbiss compressed air spray tip to deliver an effective rate of 280 g/ha glufosinate per application. 10 mL of the glufosinate herbicide solution was pipetted into a 20 mL scintillation vial for each tray to be sprayed. The spray was delivered using a horizontal and vertical application pattern. After each spray, a spray label with the herbicide name, application rate, and application date was added to each selection tray. 4 to 7 days after the second spray, herbicide-resistant plants were identified and transplanted into pots prepared with Sunshine mix LP5. Transplanted plants were placed in a greenhouse with the above mentioned growth conditions. Six to eight weeks after transplanting, the seed from each plant was harvested and stored separately with a unique identification number.

### Example 7: Agrobacterium-Mediated Transformation of Canola

Agrobacterium Preparation: Agrobacterium strains containing either pDAB7319, pDAB7321, pDAB7324, pDAB7326, pDAB7328, pDAB7330 or pDAB7331 were used to streak YEP (Bacto Peptone (20.0 g/L) and Yeast Extract (10.0 g/L)) plates containing streptomycin (100 mg/mL) and spectinomycin (50 mg/mL), and incubated for 2 days at 28° C. A loop of the 2-day streak plate was inoculated into 150 mL modified YEP liquid with streptomycin (100 mg/mL) and spectinomycin (50 mg/mL) into sterile 500 mL baffled flask(s) and shaken at 200 rpm at 28° C. The cultures were resuspended in M-medium (LS salts; 3% glucose; modified B5 vitamins; 1 μM kinetin; 1 μM 2,4-D; pH 5.8), and diluted to the appropriate density (50 Klett Units), prior to transformation of canola hypocotyls.

Canola Transformation:

Seed Germination:

Canola seeds (variety Nexera 710) were surface-sterilized in 10% Clorox for 10 minutes, and rinsed in steel strainers three times with sterile distilled water. Seeds were planted for germination on ½ MS Canola medium (½ MS, 2% sucrose, 0.8% Agar) contained in Phytatrays (25 seeds per Phytatray). The trays were placed in an environmental growth chamber (Percival Scientific, Inc., Perry, Iowa) with a growth regime set at 25° C. and a photoperiod of 16-hours light/8-hours dark, and germinated for 5 days.

Pre-Treatment:

On day 5, ˜3 mm hypocotyl segments were aseptically excised, discarding the root and shoot sections (drying of hypocotyls was prevented by placing them into 10 mL of sterile milliQ water during the excision process). Hypocotyl segments were placed horizontally on sterile filter paper on callus induction medium, MSK1D1 (MS; 1 mg/L Kinetin; 1 mg/L 2,4-D; 3% sucrose; 0.7% Phytagar) for 3 days pre-treatment in an environmental growth chamber with a growth regime set at 22-23° C. and a photoperiod of 16-hours light/8-hours dark.

Co-Cultivation with Agrobacterium:

The day before Agrobacterium treatment, flasks of YEP medium containing the appropriate antibiotics were inoculated. Hypocotyl segments were transferred from filter paper to empty 100×25 mm petri dishes containing 10 mL of liquid M medium to prevent the hypocotyl segments from drying. A spatula was used at this stage to scoop the segments and transfer. The liquid M medium was removed with a pipette, and 40 mL of Agrobacterium suspension was added to the petri dish (500 segments with 40 mL of Agrobacterium solution). The segments were treated for 30 minutes with periodic swirling of the petri dish so that the hypocotyls stayed immersed in the Agrobacterium solution. At the end of the treatment period, the Agrobacterium solution was pipetted into a waste beaker, autoclaved, and discarded (the Agrobacterium solution was completely removed to prevent Agrobacterium overgrowth). The treated hypocotyls were transferred with forceps back to the original plates containing MSK1D1 with filter paper, with care taken to ensure that the segments did not dry. The hypocotyl segments, along with control segments, were returned to the an environmental growth chamber under reduced light intensity (by covering the plates with aluminum foil), and the treated hypocotyls were co-cultivated with Agrobacterium for 3 days.

Callus Induction on Selection Medium:

After 3 days of co-cultivation, the hypocotyl segments were transferred individually with forceps onto callus induction medium, MSK1D1H1 (MS; 1 mg/L Kinetin; 1 mg/L 2,4-D; 0.5 g/L MES; 5 mg/L AgNO3; 300 mg/L Timentin; 200 mg/L Carbenicillin; 1 mg/L Herbiace; 3% sucrose; 0.7% Phytagar). The hypocotyl segments were anchored on the medium, but were not embedded in the medium.

Selection and Shoot Regeneration:

After 7 days on callus induction medium, the callusing hypocotyl segments were transferred to Shoot Regeneration Medium 1 with selection, MSB3Z1H1 (MS; 3 mg/L BAP; 1 mg/L Zeatin; 0.5 g/L MES; 5 mg/L AgNO3; 300 mg/L Timentin; 200 mg/L Carbenicillin; 1 mg/L Herbiace; 3% sucrose; 0.7% Phytagar). After 14 days, the hypocotyls with shoots were transferred to Regeneration Medium 2 with increased selection, MSB3Z1H3 (MS; 3 mg/L BAP; 1 mg/L Zeatin; 0.5 gm/L MES; 5 mg/L AgNO3; 300 mg/L Timentin; 200 mg/L Carbenicillin; 3 mg/L Herbiace®; 3% sucrose; 0.7% Phytagar).

Shoot Elongation:

After 14 days, the segments with shoots were transferred to shoot elongation medium, MSMESH5 (MS; 300 mg/L Timentin; 5 mg/L Herbiace®; 2% sucrose; 0.7% TC Agar). Shoots that were already elongated were isolated and transferred to MSMESH5. After 14 days, the remaining shoots that had not elongated in the first round were placed on MSMESH5, and transferred to fresh selection medium of the same composition. At this stage, all remaining hypocotyl segments were discarded.

Shoots that elongated on MSB3Z1H3 medium after 2 weeks were isolated and transferred to MSMESH5 medium. Remaining shoots that had not elongated in the first round on MSMESH5 were isolated, and transferred to fresh selection medium of the same composition. At this stage all remaining hypocotyl segments were discarded.

Root Induction:

After 14 days, the shoots were transferred to MSMEST medium (MS; 0.5 g/L MES; 300 mg/L Timentin; 2% sucrose; 0.7% TC Agar) for root induction. The shoots that did not root in the first transfer on MSMEST medium were transferred for a second or third cycle on MSMEST medium until rooted plants were obtained. The shoots that did not elongate/root in the first transfer on MSMEST medium were transferred for a second or third cycle on MSMEST medium until rooted plants were obtained. Plants that rooted on MSMESH5 or MSMEST and were PCR-positive were sent for transplanting into soil. After hardening, the T₀ canola plants were further analyzed for events which contained the transgene PTU cassettes. Plants were then transferred to a greenhouse, grown to maturity, and the seed was harvested for additional analysis.

### Example 8: DNA Analysis of T₁ Arabidopsis Leaf Tissue and T₀ Canola Leaf Tissue

T₀ canola plants and T₁ Arabidopsis plants were analyzed to identify plants which contained the PTU expression cassettes. Invader® assays were performed to initially screen samples of putatively transformed plants, and identify events which contained a single copy of the pat PTU. Events that were identified as single copy events were kept and further analyzed for the presence of the desaturase PTU(s) via PCR. Events that were PCR positive for the desaturase expression cassette PTU(s) were further analyzed via Southern blot analysis. Southern blot analysis was completed to confirm that the plants contained the gene expression cassette PTUs from the binary vector used to transform the plants. Single copy events containing all of the PTUs were selected for advancement.

DNA Isolation:

Total genomic DNA (gDNA) was extracted from lyophilized leaf tissue using Qiagen's DNeasy® 96 Plant Kit (Qiagen, Valencia, Calif.). This gDNA was then diluted to 10 ng/μL (canola) or 0.7 ng/μL (Arabidopsis) for use in PCR and Invader® assays for copy number.

Invader® Analysis:

Copy number analysis of the selectable marker, pat, was completed using the Invader® assay (Third Wave Technologies, Madison, Wis.). Genomic DNA was denatured at 95° C. for 10 minutes, chilled on ice, and mixed with a master mix of reagents containing oligonucleotide probes, dye molecules capable of fluorescence resonance energy transfer (FRET), and cleavase enzyme, according to the manufacturer's recommended protocol. The reactions contained probes for the internal reference genes. The 1-deoxyxylulose-5-phosphate reductoisomerase (DXR1) gene was used as an internal reference gene for Arabidopsis Invader® assay reactions, and high mobility group protein gene (HMGa) was used as an internal reference gene for canola Invader® assay reactions. In addition, the plates contained 1 copy, 2 copy, and 4 copy standards, as well as wild-type control samples and blank wells containing no sample. The whole reaction was overlayed with mineral oil before incubation in a thermocycler at 63° C. for 1.5 hrs. The resulting reaction was read on a fluorometric plate reader (Synergy™ 2, BioTek Instruments, Winooski, Vt.). Readings were collected for both FAM (λ485-528 nm) and RED (λ560-620 nm) channels. From these, the fold-over-zero (i.e., background) for each channel was determined for each sample by dividing the sample raw signal by the no template raw signal. From this data, a standard curve was constructed, and the best fit was determined by linear regression analysis. Using the parameters identified from this fit, the apparent pat copy number was then determined for each sample.

PCR Analysis:

PCR analysis was completed using primers which amplified each plant transcription unit. These primers were located in the promoter (Phaseolin) and the 3′ UTR (Phaseolin or ORF23). These same primer sets were used for PCR analysis of both canola and Arabidopsis. For PCR analysis of pDAB7319 and pDAB7324 events, primers MAS414 (SEQ ID NO: 18) and MAS415 (SEQ ID NO: 19) were used to amplify the first PTU. This PTU consisted of the Phaseolin promoter, a functional equivalent of an acyl-CoA delta-9 desaturase gene from Aspergillus nidulans (AnD9DS v3; SEQ ID NO:49), and the Phaseolin 3′UTR terminator. For PCR amplification of the second PTU in construct pDAB7319, primers MAS415 and MAS413 (SEQ ID NO: 20) were used. This PTU consists of the Phaseolin promoter, a functional equivalent of an acyl-CoA delta-9 desaturase gene from Leptosphaeria nodorum (LnD9DS-2 v2; SEQ ID NO:17), and the ORF23 3′UTR. The MAS415 and MAS413 primer pairs were also used to amplify the second PTU of events generated by transformation with pDAB7324 (Phaseolin promoter, Helicoverpa zea acyl-CoA delta-9 desaturase gene v2 (HzD9DS v2; SEQ ID NO:16), and ORF23 3′UTR). In addition, MAS415 and MAS413 primer pairs were used to amplify the PTUs in constructs pDAB7321 and pDAB7326.

The PCR reactions were carried out in 25 μL volumes using 20 ng genomic DNA, 5 units Ex Taq (Takara), lx reaction buffer, 0.2 μM of each dNTP, and 0.8 μM of each primer. The amplification reactions were performed in a DNA Engine Tetrad® 2 thermal cycler (BioRad, Hercules, Calif.). The following cycling conditions were used for primers MAS413 and MAS415: 3 minutes at 94° C.; followed by 35 cycles of 30 sec at 94° C., 30 sec at 63° C., and 3 min at 72° C.; and a final extension of 10 minutes at 72° C. The cycling conditions used for primers MAS414 and MAS415 were the same with the sole difference that the annealing temperature was reduced from 63° C. to 60° C. The reaction products were run on a 1% agarose gel, stained with ethidium bromide, and visualized on a Gel-Doc™

Southern Blot Analysis:

Southern blot analysis was used to establish the integration pattern of the canola events. These experiments generated data which demonstrated the integration and integrity of the desaturase transgene within the canola genome. Selected events were characterized as a full-length, simple integration event containing a single copy of the desaturase transgene from the binary vector used for plant transformation.

Detailed Southern blot analysis was conducted using probes specific to the desaturase genes and descriptive restriction enzymes, which cleaved at sites located within the plasmid. These digests produced hybridizing fragments internal to the plasmid, or fragments that spanned the junction of the plasmid with canola genomic DNA (border fragments). The molecular sizes indicated from the Southern hybridization for the combination of the restriction enzymes and the probes were unique for each event. These analyses also showed that the plasmid fragment had been inserted into canola genomic DNA without rearrangements of the T-strand DNA.

For Southern blot analysis, 100 mg of lyophilized canola leaf tissue was extracted using the Plant Mini Kit (Qiagen). Five micrograms (5 μs) of gDNA per sample was digested simultaneously with SpeI and PacI restriction endonucleases (New England Biolabs, Ipswich, Mass.) to obtain fragments containing either the PTUs of interest, and/or the selectable marker (PAT), to determine copy number. The digested DNA was separated on a 0.8% agarose gel.

Briefly, following electrophoretic separation and visualization of the DNA fragments, the gels were depurinated with 0.25N HCl for approximately 20 minutes, and then exposed to a denaturing solution for approximately 30 minutes, followed by a neutralizing solution for at least 30 minutes. Southern transfer was performed overnight onto nylon membranes (Millipore, Billerica, Mass.) using a wicking system with 10×SSC. After transfer, the membranes were washed with a 2×SSC solution, and the DNA was bound to the membrane by UV crosslinking. This process produced Southern blot membranes ready for hybridization.

Probes were generated and PCR fragments were amplified from plasmid DNA and purified via gel extraction using the QIAquick® Gel Extraction kit (Qiagen). The primers used to create the LnD9DS probe were arw008 (SEQ ID NO:21) and arw009 (SEQ ID NO:22). The primers used to create the HzD9 probe were arw010 (SEQ ID NO:23) and arw011 (SEQ ID NO:24). PCR conditions for all three reactions consisted of 35 cycles with an annealing temperature of 63° C. and an extension time of 1 minute. The PCR fragments were labeled with ³²P using the Prime-It® RmT Random Primer Labeling kit (Stratagene, La Jolla, Calif.).

The hybridization step was conducted at approximately 65° C. overnight in the hybridization oven. The nylon membrane blots were rinsed, and the blot was exposed on a phosphor image screen overnight, and scanned on a Storm™ 860 Scanner (Molecular Dynamics, Sunnyvale, Calif.).

### Example 9: Fatty Acid Composition of Seeds from Transgenic Arabidopsis Containing an Acyl-CoA Delta-9 Desaturase

Arabidopsis plants were transformed with Agrobacterium vectors containing genes for LnD9DS-2 v2 (pDAB7321; SEQ ID NO:61), HzD9DS v2 (pDAB7326; SEQ ID NO:63) or MgD9DS v2 (pDAB7330; SEQ ID NO:65). Plants were also transformed with a vector containing a AnD9DS gene (pDAB7328; SEQ ID NO:64). An empty vector containing only the selectable marker pat gene (pDAB7331; SEQ ID NO:66) was used as a negative control. Transformations were also performed using two desaturases in combination, to combine a stearoyl-preferring desaturase (AnD9DS) with a palmitoyl-preferring desaturase, either LnD9DS-2 (pDAB7319; SEQ ID NO:60), or HzD9DS (pDAB7324; SEQ ID NO:62). In all cases, the desaturase genes were driven by the seed-specific PvPhas promoter (U.S. Pat. No. 5,504,200). Bulk T₂ seed was harvested from herbicide-resistant T₁ plants that were confirmed to contain the pat gene by Invader® assay analysis and the desaturase PTU by PCR analysis.

Seed samples were homogenized in heptane-containing triheptadecanoin (Nu-Chek prep, Elysian, Minn.) as a surrogate using a steel ball and ball mill. Prior to homogenization, a solution of 0.25 M freshly-prepared MeONa (Sigma) in MeOH was added to the sample. The reaction was conducted under mild heat (40° C.) and constant shaking. The reaction was verified by the recovery of the methylated surrogate. Extraction of FAMEs was repeated three times, and all heptane layers were pooled prior to analysis. The completeness of the extraction was verified by checking for the presence of FAMEs in a fourth extraction/derivatization. The resulting FAMEs were analyzed by GC-FID using a capillary column BPX 70 from SGE (15 m×0.25 mm×0.25 μm). Each FAME was identified by retention time, and quantified by the injection of a rapeseed oil reference mix from Matreya, LLC (Pleasant Gap, Pa.), as a calibration standard.

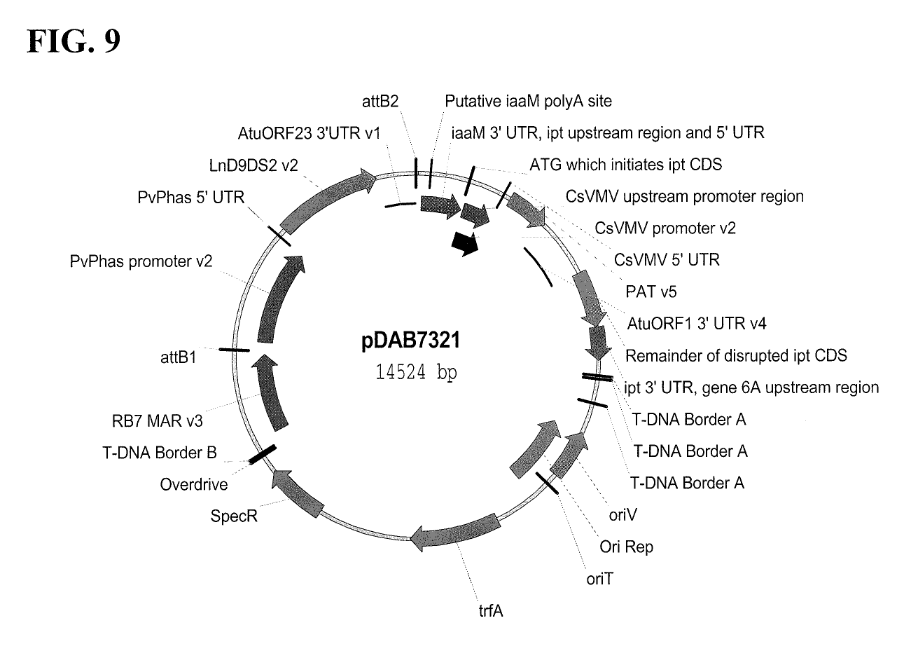

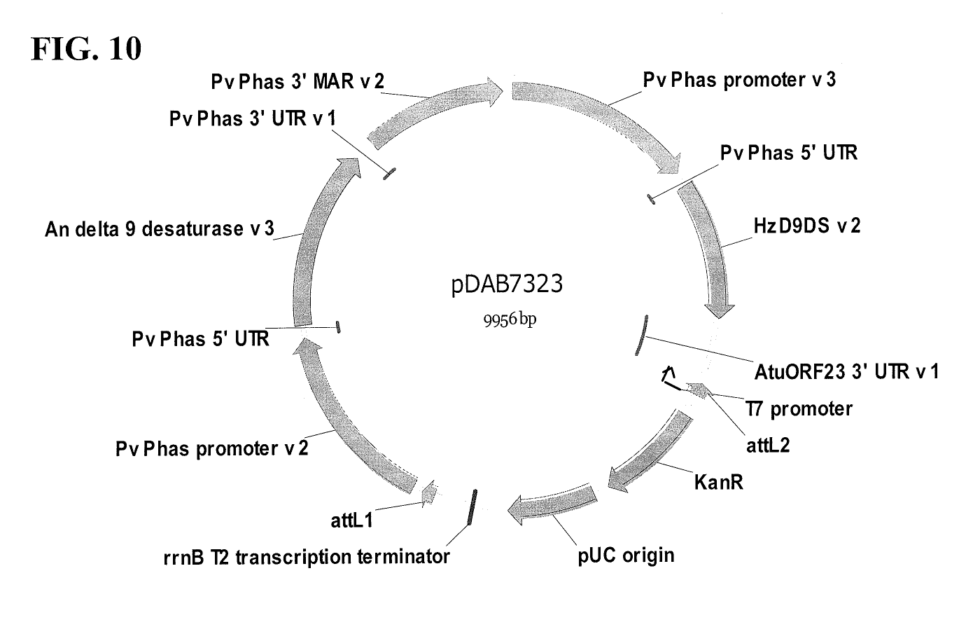

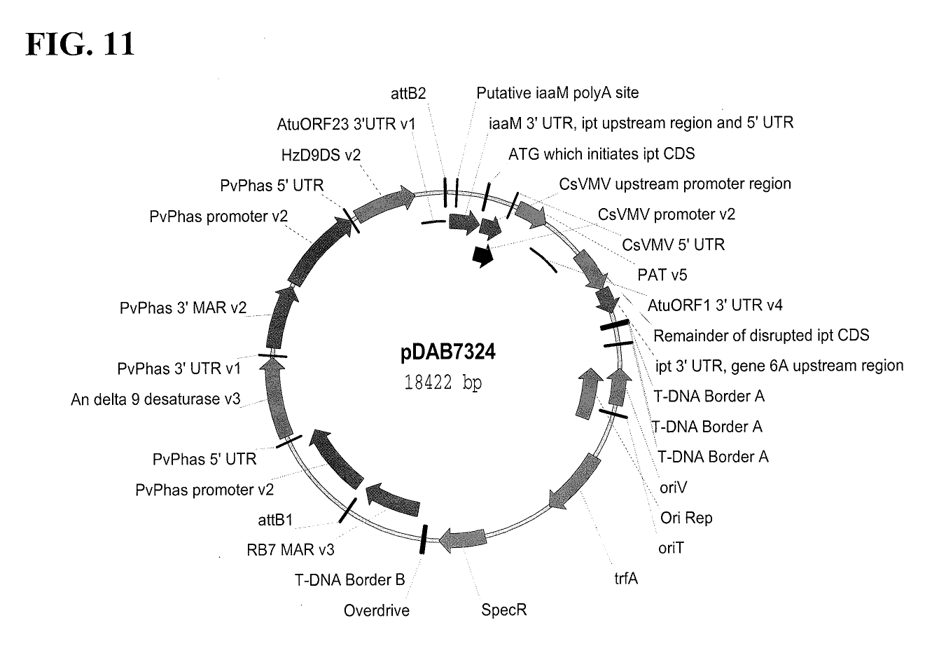

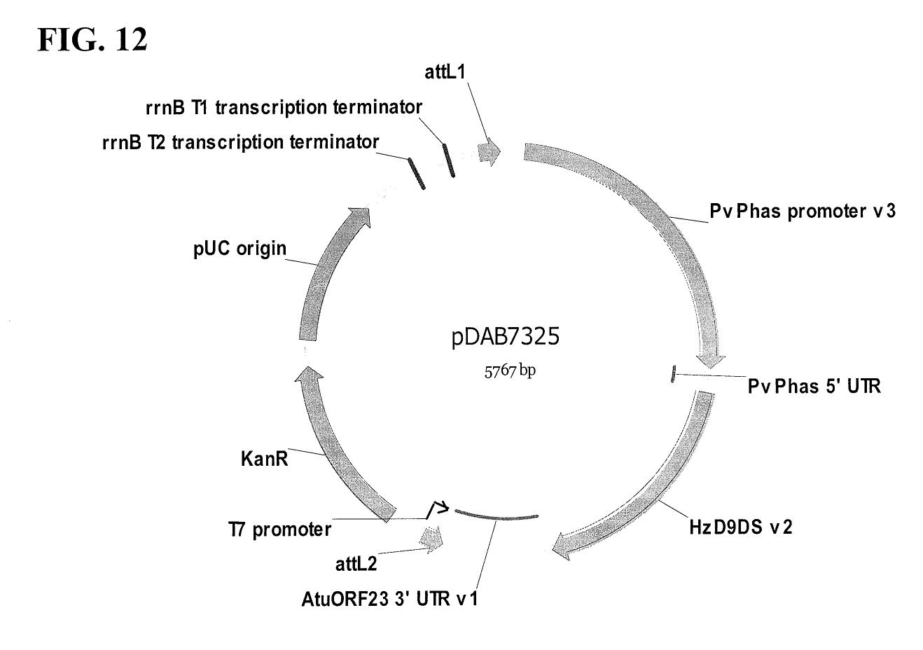

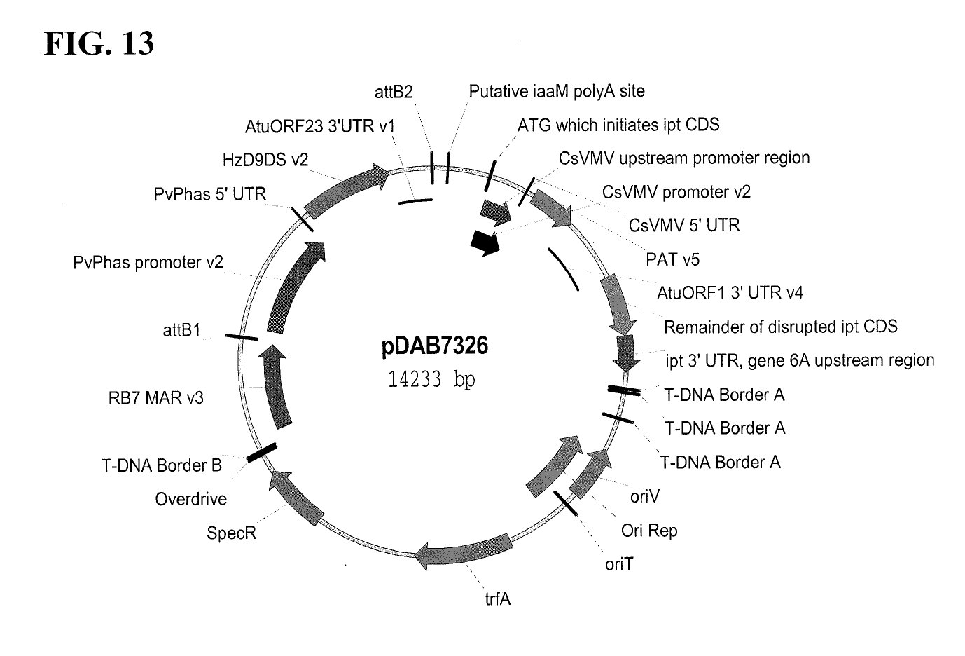

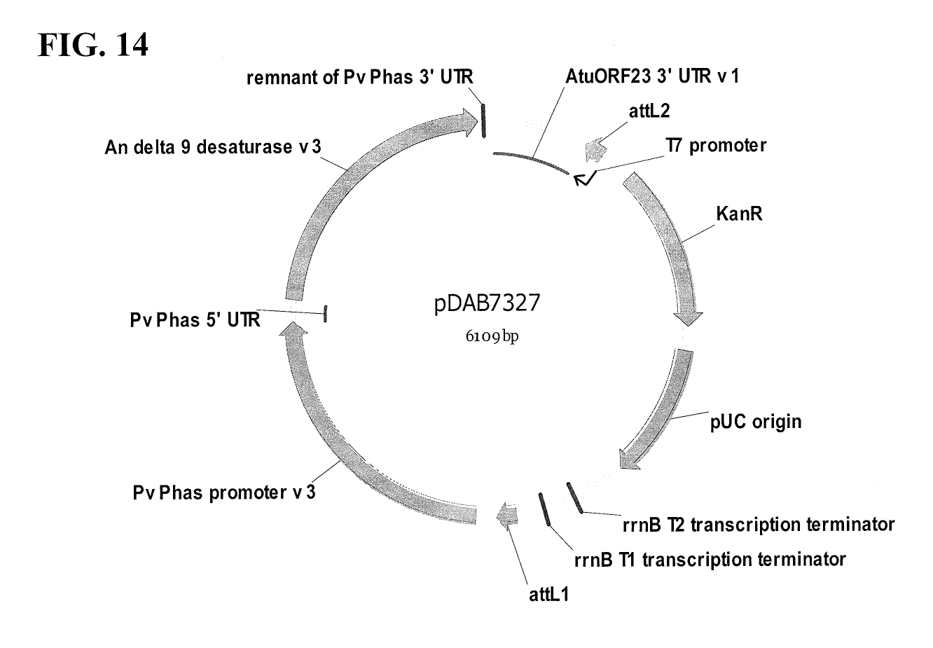

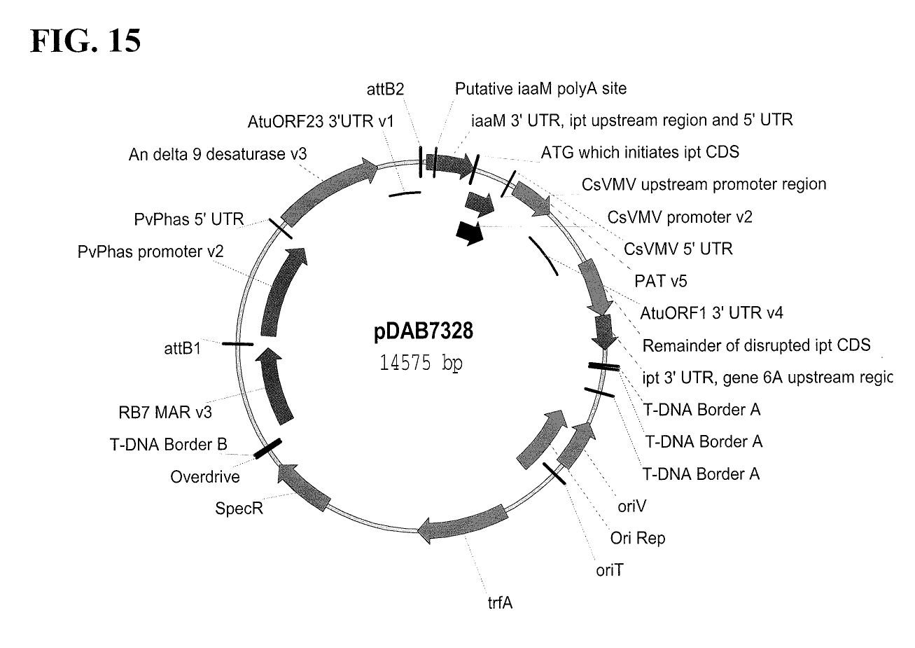

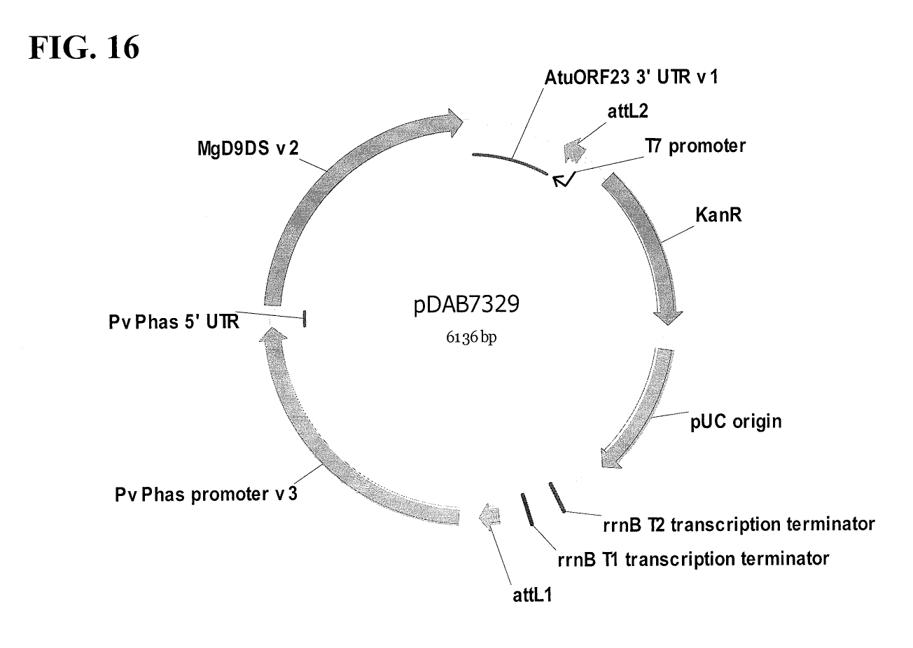

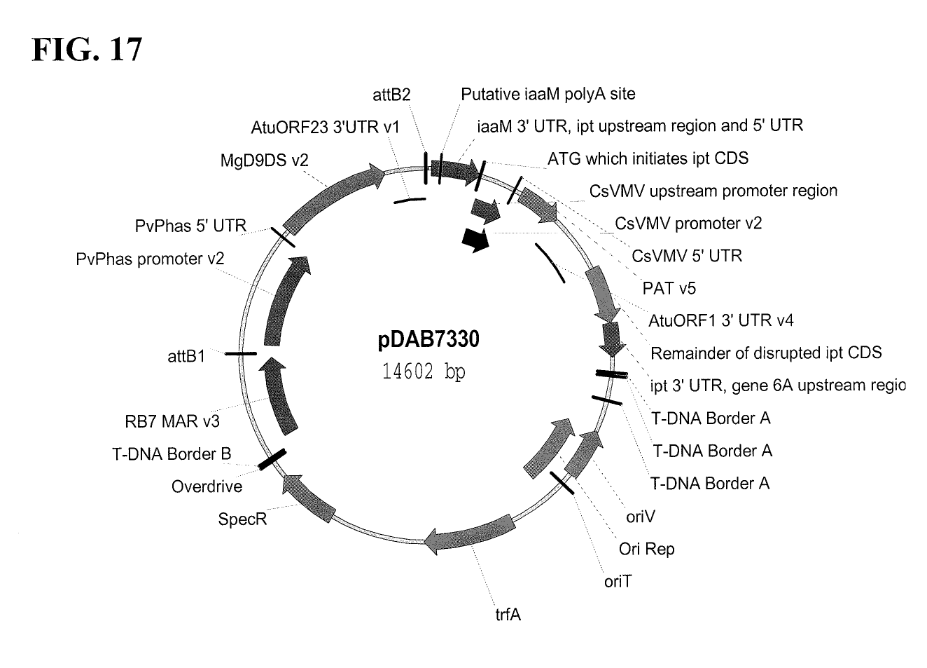

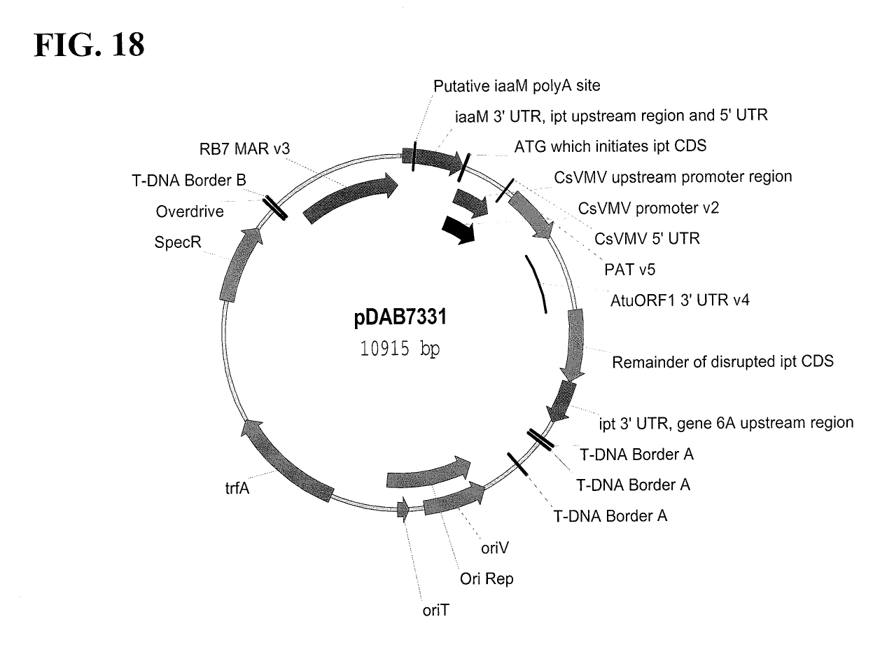

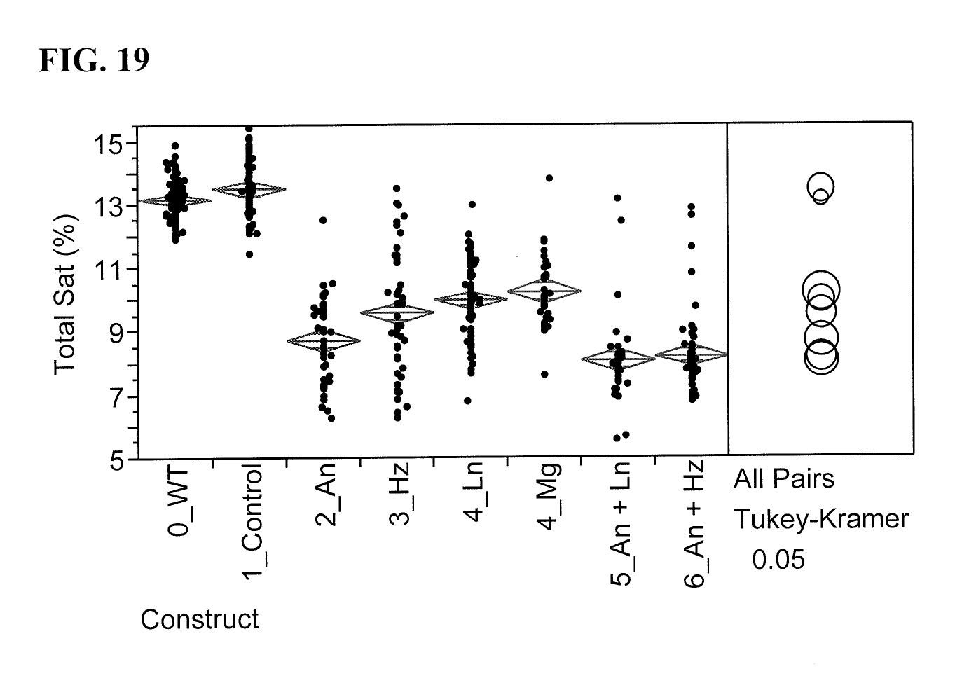

FAME analysis of T₂ seed from the transgenic events showed that expression of each of the desaturases had a significant effect on reducing the total saturated fatty acid content of the seeds, as determined from the mean saturated fatty acid content of each set of events. Table 10 and FIG. 19. In this table and the following tables, the values not connected by the same letter are significantly different, as determined using the Tukey-Kramer HSD test performed in the IMP® statistical software package (SAS Institute Inc., Cary, N.C.). Combinations of AnD9DS with LnD9DS-2 or HzD9DS yielded the lowest mean total saturated fatty acid content.

TABLE 10
Total saturated fatty acid content of T₂ Arabidopsis seed

| | Number of | | | | Mean Total |
|---|---|---|---|---|---|
| Gene | T2 samples | | | | Saturated FAs |
| Control | 204 | A | | | 13.49 |
| WT | 60 | A | | | 13.16 |
| MgD9DS v2 | 42 | | B | | 10.26 |
| LnD9DS-2 v2 | 49 | | B | | 10.00 |
| HzD9DS v2 | 70 | | B | | 9.58 |
| AnD9DS v3 | 32 | | | C | 8.73 |
| AnD9DS v3 + | 39 | | | C | 8.23 |
| HzD9DS v2 | | | | | |
| AnD9DS v3 + | 51 | | | C | 8.09 |
| LnD9DS-2 v2 | | | | | |

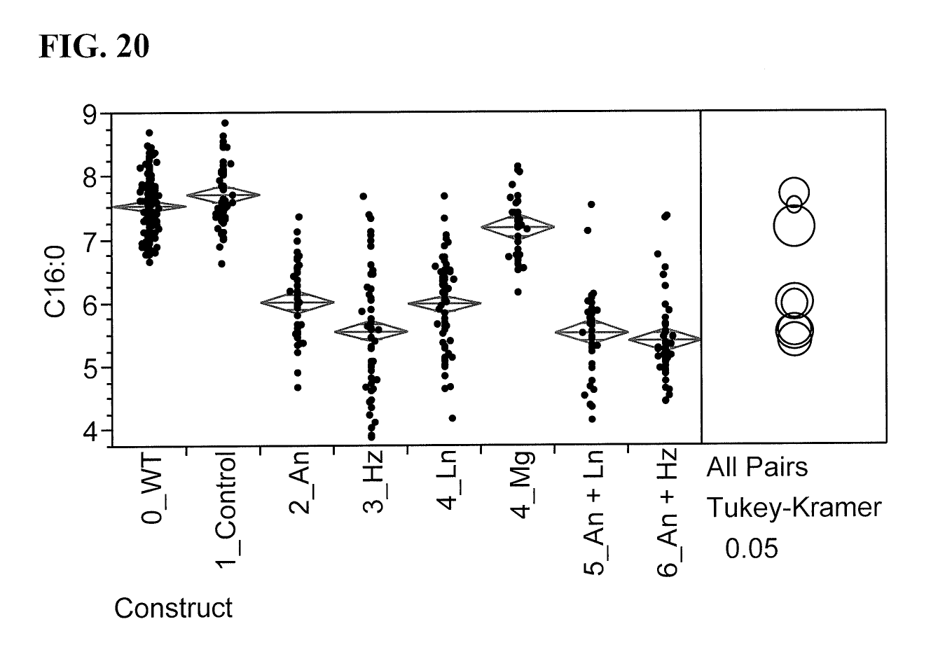

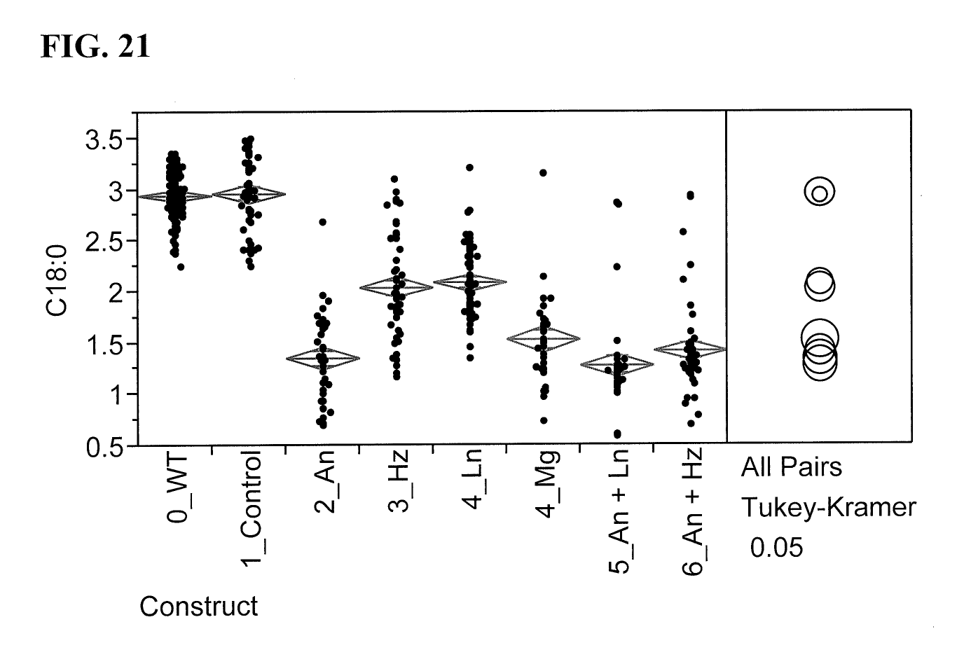

Although the desaturases all lowered the total saturated fatty acid content in Arabidopsis seeds, they had different effects on the palmitic and stearic acid fatty acid contents, as predicted from the yeast experiments. Table 11 and FIG. 20 show the mean palmitic acid content for each set of events. Table 12 and FIG. 21 show the mean stearic acid content of T₂ seed for each set of events.

TABLE 11
Palmitic acid content of T₂ Arabidopsis seed

| | | | | | Mean |
|---|---|---|---|---|---|
| Gene | | | | | Palmitic acid |
| Control | A | | | | 7.72 |
| WT | A | | | | 7.54 |
| MgD9DS v2 | | B | | | 7.19 |
| AnD9DS v3 | | | C | | 6.02 |
| LnD9DS-2 v2 | | | C | | 5.98 |
| HzD9DS v2 | | | | D | 5.57 |
| AnD9DS v3 + | | | | D | 5.54 |
| LnD9DS-2 v2 | | | | | |
| AnD9DS v3 + | | | | D | 5.41 |
| HzD9DS v2 | | | | | |

TABLE 12
Stearic acid content of T₂ Arabidopsis seed

| | | | | Mean |
|---|---|---|---|---|
| Gene | | | | Stearic acid |
| Control | A | | | 2.96 |
| WT | A | | | 2.94 |
| LnD9DS-2 v2 | | B | | 2.09 |
| HzD9DS v2 | | B | | 2.04 |
| MgD9DS v2 | | | C | 1.53 |
| AnD9DS v3 + | | | C | 1.42 |
| HzD9DS v2 | | | | |
| AnD9DS v3 | | | C | 1.35 |
| AnD9DS v3 + | | | C | 1.28 |
| LnD9DS-2 v2 | | | | |

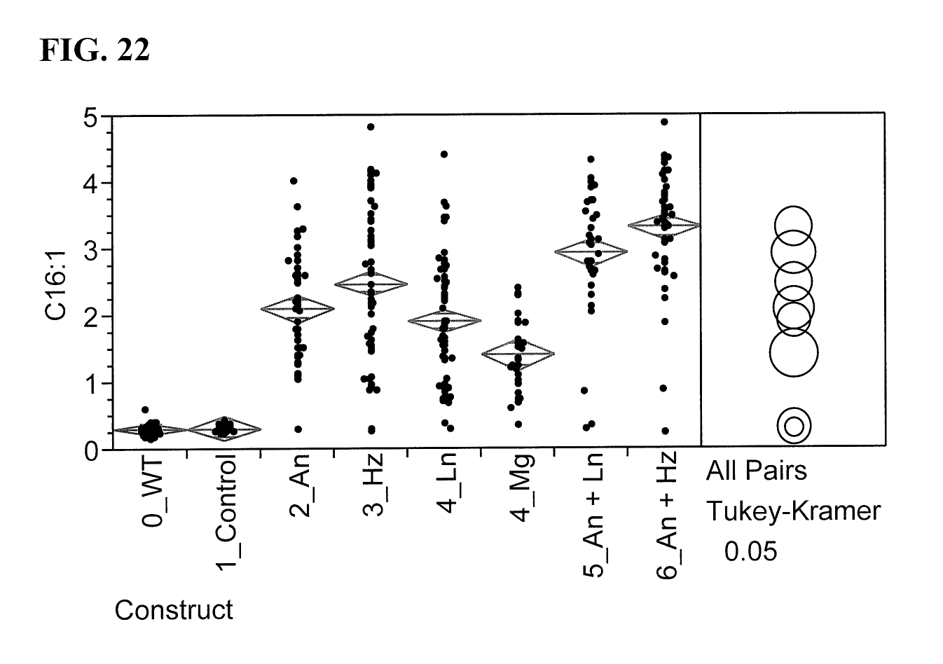

AnD9DS and MgD9DS had greater effects on the stearic acid content than LnD9DS-2 and HzD9DS. Conversely, LnD9DS-2 and HzD9DS had greater effects on the palmitic content than AnD9DS and MgD9DS. Combinations of the desaturases have the greatest effect on both fatty acids. These results were also observed in the effects of the desaturases on increasing the seed content of palmitoleic acid, which is the primary product of delta-9 desaturation of palmitic acid. Table 13 and FIG. 22.

TABLE 13
Palmitoleic acid content of T₂ Arabidopsis seed

| | | | | | | Mean Palmitoleic |
|---|---|---|---|---|---|---|
| Gene | | | | | | Acid |
| AnD9DS v3 + | A | | | | | 3.32 |
| HzD9DS v2 | | | | | | |
| AnD9DS v3 + | A | | | | | 2.93 |
| LnD9DS-2 v2 | | | | | | |
| HzD9DS v2 | | B | | | | 2.48 |
| AnD9DS v3 | | B | C | | | 2.10 |
| LnD9DS-2 v2 | | | C | | | 1.91 |
| MgD9DS v2 | | | | D | | 1.40 |
| Control | | | | | E | 0.31 |
| WT | | | | | E | 0.30 |

There was expected variation in the effect of the desaturases on saturated fatty acid content across the events analyzed, due to position and copy number effects. A comparison of the complete fatty acid profile of events with the lowest total saturated fatty acid content (average of the five lowest events) is shown in Table 14 alongside the profile of seed from wild-type and control-transformed plants.

TABLE 14
Fatty acid profile of T₂ transgenic Arabidopsis with lowest total saturated fatty acid
content. Standard deviations are in parentheses.

| | C14:0 | C16:0 | C16:1 | C18:0 | C18:1 | Vacc.* |
|---|---|---|---|---|---|---|
| WT | 0.08 | 7.54 | 0.31 | 2.94 | 14.91 | 1.47 |
| | (0.02) | (0.41) | (0.05) | (0.19) | (1.44) | (0.10) |
| Control | 0.08 | 7.72 | 0.32 | 2.96 | 14.20 | 1.46 |
| | (0.02) | (0.05) | (0.04) | (0.34) | (2.04) | (0.11) |
| AnD9DS v3 | 0.07 | 5.10 | 2.92 | 0.72 | 20.52 | 1.72 |
| | (0.01) | (0.38) | (0.55) | (0.03) | (2.12) | (0.26) |
| HzD9DS v2 | 0.06 | 4.13 | 4.11 | 1.26 | 19.34 | 1.94 |
| | (0.00) | (0.23) | (0.47) | (0.08) | (1.01) | (0.25) |
| LnD9DS-2 v2 | 0.05 | 4.68 | 3.49 | 1.53 | 19.35 | 2.05 |
| | (0.00) | (0.30) | (0.69) | (0.12) | (0.81) | (0.21) |
| MgD9DS v2 | 0.08 | 6.64 | 1.60 | 1.05 | 18.01 | 1.60 |
| | (0.02) | (0.26) | (0.54) | (0.20) | (1.86) | (0.16) |
| AnD9DS v3 + | 0.06 | 4.41 | 3.71 | 0.97 | 19.60 | 2.03 |
| LnD9DS-2 v2 | (0.00) | (0.17) | (0.35) | (0.33) | (0.88) | (0.21) |
| AnD9DS v3 + | 0.08 | 4.86 | 4.09 | 1.01 | 18.10 | 2.03 |
| HzD9DS v2 | (0.02) | (0.35) | (0.65) | (0.22) | (2.40) | (0.31) |

| | C18:2 | C18:3 | C20:0 | C20:1 | C20:2 | C22:0 | C22:1 | C24:0 |
|---|---|---|---|---|---|---|---|---|
| WT | 28.72 | 17.85 | 2.06 | 20.11 | 1.78 | 0.34 | 1.68 | 0.21 |
| | (0.97) | (0.81) | (0.16) | (0.90) | (0.15) | (0.10) | (0.19) | (0.10) |
| Control | 29.28 | 18.07 | 2.08 | 19.62 | 1.85 | 0.39 | 1.70 | 0.27 |
| | (1.29) | (1.35) | (0.16) | (1.23) | (0.17) | (0.13) | (0.04) | (0.14) |
| AnD9DS v3 | 29.64 | 17.59 | 0.44 | 18.26 | 1.42 | 0.24 | 1.26 | 0.10 |
| | (1.34) | (1.28) | (0.04) | (0.83) | (0.15) | (0.16) | (0.09) | (0.05) |
| HzD9DS v2 | 29.31 | 17.26 | 0.81 | 18.39 | 1.47 | 0.18 | 1.50 | 0.23 |
| | (0.94) | (0.39) | (0.06) | (0.66) | (0.10) | (0.05) | (0.04) | (0.03) |
| LnD9DS-2 v2 | 27.72 | 17.46 | 1.00 | 19.33 | 1.45 | 0.32 | 1.48 | 0.10 |
| | (0.18) | (0.55) | (0.11) | (0.46) | (0.11) | (0.14) | (0.10) | (0.09) |
| MgD9DS v2 | 29.76 | 17.98 | 0.63 | 19.19 | 1.60 | 0.26 | 1.44 | 0.16 |
| | (1.10) | (0.84) | (0.63) | (0.86) | (0.09) | (0.20) | (0.09) | (0.03) |
| AnD9DS v3 + | 29.17 | 18.84 | 0.59 | 17.65 | 1.40 | 0.39 | 1.13 | 0.03 |
| LnD9DS-2 v2 | (0.31) | (0.41) | (0.27) | (0.23) | (0.04) | (0.03) | (0.06) | (0.02) |
| AnD9DS v3 + | 29.28 | 18.83 | 0.65 | 17.88 | 1.55 | 0.20 | 1.33 | 0.11 |
| HzD9DS v2 | (1.78) | (1.69) | (0.21) | (1.90) | (0.20) | (0.12) | (0.24) | (0.08) |

*Vacc. = cis-vaccenic acid (18:1 n-7)

In addition to reducing the content of the saturated palmitic and stearic fatty acids, and increasing the monounsaturated fatty acid content (palmitoleic and oleic), the presence of the desaturases also lowered the amount of arachidic acid (C20:0) in the seeds. This is presumably because this fatty acid is derived from elongation of stearic and palmitic acids. There appeared to be no direct desaturation of C20:0 by the introduced desaturases, as there is no concomitant rise in eicosenoic acid (C20:1) as C20:1Δ9.

### Example 10: Delta-9 Desaturase Antibody Preparation

Diagnostic tools such as antibodies are desirable to characterize transgenic delta-9 desaturase protein expression in plants. Because acyl-CoA delta-9 desaturases are membrane-bound proteins, routine over-expression in Escherichia coli is difficult. However, antibodies were successfully generated by over-expression of a C-terminal fragment of each delta-9 desaturase protein that does not include any of the transmembrane domains of the protein.

Polymerase Chain Reactions:

PCR primers were designed to amplify an equivalent C-terminal fragment for each desaturase. The 3′ primer was designed to encode a protein fragment with a C-terminal 6×His tag. Ndel and BamHI restriction sites were incorporated into the 5′ and 3′ primers, respectively, to facilitate cloning. The primer sequences are given below in Table 15. The expected amplification products were 659 bp for LnD9DS-2, 683 bp for MgD9DS, and 335 bp for HzD9DS. PCR reactions were carried out using the Takara Ex Taq™ PCR kit (Clontech, Mountain View, Calif.) using supplier conditions. The total PCR reaction volume was 50 μL. Each reaction contained 200 ng of plasmid DNA and 50 pmol of each primer. The DNA was denatured at 94° C. for 1 min, followed by 30 cycles of 94° C. for 30 sec, 60° C. for 1 minute, and 72° C. for 30 sec. A final extension was carried out at 72° C. for 10 minutes. Each PCR product was run across two wells on a sterile 0.75% agarose gel, and DNA was gel purified using Montage spin columns and eluted in 15 μL TE buffer.

TABLE 15
Sequences of the oligonucleotide primers used in the PCR
amplifications of C-terminal fragments from LnD9DS-2,
MgD9DS, and HzD9DS.

| Primer | Sequence | Purpose |
|---|---|---|
| AntiLnD9DS2F | SEQ ID NO: 25 | Forward primer for |
| | CATATGTTCGACGACAGACGCACGCCTCGAGAC | LnD9DS2 C-terminal |
| AntiLnD9DS2Rh | SEQ ID NO: 26 | Reverse primer for |
| | GGATCCGCAGCCACAGCCCCCTCAACCAACCTCTC | LnD9DS2 C-terminal |
| AntiMgD9DSF | SEQ ID NO: 27 | Forward primer for |
| | CATATGTTCGACGATCGCAACTCGCCGCGTGATCAC | MgD9DS C-terminal |
| AntiMgD9DSRh | SEQ ID NO: 28 | Reverse primer for |
| | GGATCCGCGGCCTGAGCACCCGGAACAGGCTG | MgD9DS C-terminal |
| AntiHzD9DSF | SEQ ID NO: 29 | Forward primer for |
| | CATATGTATGACAAGTCCATCAAGCCTTCC | HzD9DS C-terminal |
| AntiHzD9DSRh | SEQ ID NO: 30 | Reverse primer for |
| | GGATCCTCGTCTTTAGGGTTGATCCTAATGGCTGC | HzD9DS C-terminal |

TOPO Cloning:

The purified C-terminal fragments were TA cloned into TOPO® pCR® 2.1 vectors (Invitrogen, Carlsbad, Calif.), and transformed into Top 10 E. coli cells following the manufacturer's protocol (Invitrogen). Transformations were selected, and plasmid DNA was purified using NucleoSpin® columns (Macherey-Nagel GmbH & Co, Duren, Germany). Three microliters (3 μL) of DNA was digested with NdeI and BamHI in a total volume of 20 μL for 90 minutes at 37° C., and run on a 0.8% agarose gel. In each case, a gene-specific fragment (plus a 3.9 kb TOPO® vector band) was visible. Three positive clones were chosen for each cloned gene and sequenced to confirm that the amplified PCR fragment was free of errors. Each of the MgD9DS clones contained a silent point mutation at base pair 45, indicating either a single nucleotide polymorphism between the published sequence and the PCR template, or a silent PCR error. Since the mutation was silent, no correction was necessary, and one clone was chosen for subcloning.

Preparation of the Delta-9 Desaturase C-Terminal Fragment Expression Plasmids:

The PCR-amplified delta-9 desaturase fragments were digested with NdeI and BamHI restriction enzymes and ligated into corresponding restriction sites within the pET30b(+) expression vector. The cloning step resulted in the addition of 15 C-terminal amino acids, constituting a C-terminal 6×His tag to facilitate full-length protein purification. These additional amino acids were not expected to affect protein expression. Positive clones were obtained and confirmed via restriction enzyme digestion and sequencing reactions.

Expression of Delta-9 Desaturase C-Terminal Peptide Fragments in E. coli:

The delta-9 desaturase/pET30b(+) expression plasmids were transformed into BL21(DE3) E. coli cells according to the manufacturer's recommended protocol (Novagen, Madison, Wis.). Cells were plated on LA plates containing kanamycin (50 μg/mL) and glucose (1.25 M). The plates were incubated overnight at 37° C. A full loop of cells was scraped from the plates, and inoculated into 500 mL flasks containing 250 mL LB and kanamycin (50 μg/mL) with isopropyl-P-D-thiogalactoside (0.75 mM) inducer. Three induction conditions were tested. Cultures were induced at different temperatures, and harvested at different times as follows: overnight (˜18 hrs) at 28° C.; overnight at 16° C.; or 4 hours at 37° C. Cells were harvested by centrifugation in 250 mL bottles at 6,000 rpm for 15 minutes, and then frozen at −20° C.

Protein Purification of Delta-9 Desaturase C-Terminal Peptide Fragments:

Cell pellets from 250 mL cultures were thawed and resuspended in 50 mL cold Phosphate Buffered Saline (PBS) containing 10% glycerol and 0.5 mL of Protease Inhibitor Cocktail (Sigma, St. Louis, Mo.) using a hand-held homogenizer. The cells were disrupted on ice for approximately 10 minutes using a Branson Model 450 Sonifier (Danbury, Conn.). Inclusion bodies were pelleted by centrifugation at 10,000×g for 15 minutes, and extracted 2-3 times with PBS containing 0.5% Triton X-100 until the protein concentration of the supernatant reached baseline, as measured by a Bradford protein assay. The recovered inclusion bodies were solubilized in a PBS solution containing 6 M Urea and 5 mM DTT at room temperature with stirring for about 1 hour. Solubilized proteins were separated from insoluble materials by centrifugation at 30,000×g for 15 minutes, and the retained supernatant was applied onto a 5 mL Ni-affinity column (GE Healthcare, HiTrap Chelating, Piscataway, N.J.). The histidine tags of the C-terminal delta-9 desaturase peptides bound to the metal resin, and each fragment was eluted with a 50-200 mM imidazole gradient using an Akta® Explorer 100 (GE Healthcare, Piscataway, N.J.). Fractions (3 mL each) were collected, and eluted peaks were analyzed by SDS-PAGE. Fractions containing C-terminal delta-9 desaturase peptide were pooled and concentrated using an Amicon® Ultra 10,000 MWCO filter device (Millipore, Billerica, Mass.) to less than 5 mL volume. The protein sample was then injected onto a Hi Load™ XK16/60 Superdex™ 200 size exclusion column (GE Healthcare, Piscataway, N.J.), and equilibrated with 6 M Urea in 20 mM Tris-HCl, 150 mM NaCl, and 1 mM DTT. The peak fractions (4 mL each) containing pure C-terminal delta-9 desaturase peptide were saved (after validation by SDS-PAGE analysis and other biochemical characterization) and used for antibody production. Peptides with the expected sizes of 27 kDa for LnD9DS-2 peptide, 15 kDa for HzD9DS peptide, and 28 kDa for MgD9DS peptide were produced. The induction conditions produced sufficient protein for visualization by Coomassie blue staining of SDS-PAGE gels.

Polyclonal Antibody Production:

A contract service (Strategic BioSolutions, Newark, Del.) produced rabbit antibodies against each of the three C-terminal delta-9 desaturase peptides. Following their standard procedures, high titer (validated by using ELISA) antisera for each of the three protein fragments was obtained. Each purified C-terminal delta-9 desaturase peptide was diluted with 20 mM Tris-HCl, 150 mM NaCl, 1 mM DTT buffer, and with a final concentration of 2-3 M urea, to keep the protein in solution. Approximately 10 mg of protein was sent to Strategic BioSolutions for generation of a polyclonal antibody. Two rabbits were chosen for each immunogen, and standard protocols (70 days immunization) were used. A new adjuvant called TiterMax® Gold was purchased for preparation of the emulsion. ELISA titration during immunization and at the end of protocol was also performed to ensure the success of antibody production. The antisera were delivered in two separate time points; one from the standard 2 month procedure, and the other from exsanguination.

To isolate total IgG from the rabbit sera, approximately 20-30 mL of high-titer antisera were applied to a 5 mL alkali-tolerant Protein A column (GE Healthcare, HiTrap™ Mab Select SuRe™, cat#11-0034-94). Following a standard wash with PBS buffer, bound IgG was eluted from the resin by short exposure to 0.1 M sodium citrate, 0.3 M NaCl, pH 3.3, and immediately neutralized by adding 1/10 volume of 2 M Tris-HCl, pH 9 buffer to each fraction. The affinity column was sanitized by treating with 0.5 N NaOH following standard cleaning-in-place (CIP) procedure to avoid cross contamination of the IgG. Final recovered IgG from each sample was dialyzed against 50 volumes of PBS at 4° C. overnight, and protein concentration was determined by Bradford assay using BSA standard (Pierce, prod#23208). One mL aliquots were transferred to individual tubes and stored at −80° C.

These antibodies are diagnostic tools that were used to measure desaturase protein expression in transgenic plant material. The antibodies were used to develop correlations between low saturated fatty acid oil phenotype changes and the level of expression of the delta-9 desaturase proteins.

### Example 11: Levels of Acyl-CoA Delta-9 Desaturase Proteins in T₂ Arabidopsis Seed

Delta-9 desaturase polypeptides were detected in mature transgenic seed samples by Western blot. Seed was prepared for analysis by cracking dry seeds with stainless steel beads in a Kleco™ Bead Beater (Garcia Machine, Visalia, Calif.). Extraction buffer was added (50 mM Tris, 10 mM EDTA, 2% SDS), and sample tubes were rocked gently for 30 minutes. Samples were centrifuged for 15 minutes at 3,000 rcf. Then, the supernatant was collected and used for analysis. The amount of total soluble protein in the seed extract was determined by Lowry assay (BioRad, Hercules, Calif.). Samples were normalized to 1.55 mg/mL total soluble protein and prepared in LDS sample buffer (Invitrogen, Carlsbad, Calif.) with 40 mM DTT, for a normalized load of 20 μg total soluble protein per lane. Samples were electrophoresed in 4-12% Bis-Tris gels (Invitrogen), and transferred to nitrocellulose membranes. Blots were blocked in blocking buffer, and probed with antibodies against four different delta-9 desaturase polypeptides (AnD9DS, LnD9DS-2, HzD9DS, and MgD9DS) (see Example 10).

In all cases, polyclonal antibody was developed in rabbits against a His-tag purified C-terminal peptide fragment of the individual desaturases as described above. The purified C-terminal fragments were used as reference antigens for quantitation of the Western blots. An anti-rabbit fluorescent labeled secondary antibody (Goat Anti-Rabbit AF 633; Invitrogen) was used for detection. Blots were visualized on a Typhoon™ Trio Plus fluorescence imager (GE Healthcare). Standard curves were generated with quadratic curve fitting, and linear regression was used to quantify expression.

SDS-PAGE Western blots of extracts from mature T₂ seed from Arabidopsis events showed bands at the appropriate size when probed with specific antisera. These bands were quantified against specific reference antigens. Quantitative Western blotting of Arabidopsis T₂ seed extracts with appropriate antiserum indicated that an average of 63 ng LnD9DS-2/mg total protein (tp) (max. 228 ng/mg tp) was detected in mature seeds, and for HzD9DS, an average of 34 ng/mg tp (max. 100 ng/mg tp) was detected. For MgD9DS, an average of 58 ng/mg tp (max. 1179 ng/mg tp) was detected in T₂ seed. For the AnD9DS events, an average of 625 ng/mg tp (max 1.5 μg/mg tp) was detected in mature T₂ seeds. Thus, there was 10-18-fold less of the palmitoyl-preferring desaturases, LnD9DS-2 and HzD9DS, expressed in the transgenic seed, relative to AnD9DS. Higher levels of expression of these desaturases would therefore drive further reductions in saturates, especially palmitic acid.

### Example 12: Expression of Delta-9 Desaturase Genes in Canola

A series of transgenic canola events were obtained from transformations performed with pDAB7321 (SEQ ID NO:61) and pDAB7326 (SEQ ID NO:63) (containing LnD9DS-2 and HzD9DS genes, respectively, driven by the seed-specific PvPhas promoter). Thirty nine pDAB7321 events containing the LnD9DS-2 gene were identified by PCR analysis of genomic DNA, and were grown in the greenhouse to produce T₁ seed. Similarly, 80 pDAB7326 events were identified that contained the HzD9DS gene, and produced T₁ seed. Canola was also transformed with pDAB7319 (SEQ ID NO:60) or pDAB7324 (SEQ ID NO:62), which contain an AnD9DS gene coupled with the LnD9DS-2 or HzD9DS genes, all driven by the PvPhas promoter. 44 and 76 events were recovered, respectively, that were confirmed to contain both desaturase genes by PCR analysis, and were grown in the greenhouse to produce T₁ seed.

FAME analysis of T₁ seed samples from events transformed with pDAB7321 (LnD9DS-2 v2) or pDAB7326 (HzD9DS v2) did not show significant reduction in saturated fatty acid levels relative to untransformed canola plants or plants transformed with an empty vector control. Western blots of the T₁ seed did not show detectable levels of the delta-9 desaturase proteins. In addition, no detectable protein for LnD9DS-2 or HzD9DS was detected in T₁ seed from plants transformed with pDAB7319 (AnD9DS v3 and LnD9DS-2 v2) or pDAB7324 (AnD9DS v3 and HzD9DS v2), whereas the AnD9DS protein could be readily detected. In these events, a reduction of saturated fatty acids was observed relative to control plants, but this was attributable to expression of AnD9DS.

To evaluate the relative mRNA levels of the delta-9 desaturase genes, total RNA was extracted from developing canola seed from events transformed with double desaturase constructs (pDAB7319 and pDAB7324) and analyzed by quantitative real-time PCR. Seeds were harvested on dry ice at 20, 25, 29, 32, 39, or 41 days after pollination from several canola plants and stored at −80° C. Total RNA was prepared from 50 mg of pooled frozen seeds using a Plant RNeasy® RNA extraction kit (Qiagen) according to the manufacturer's recommended protocol. Extracted RNA was used as a template for cDNA synthesis using the SuperScript® III First Strand Synthesis Supermix for qRT-PCR (Invitrogen) according to the manufacturer's recommended protocol.

RT-PCR assays were designed against the desaturase targets using the Roche Assay Design Center (Roche Diagnostics, Indianapolis, Ind.). Primers used in the assay are described in Table 16. Target assays utilized FAM-labeled UPL probes (Roche Diagnostics). These assays were executed in duplex reactions with a Texas-Red-labeled canola actin reference assay synthesized by Integrated DNA Technologies.

TABLE 16
q-RT-PCR assay details

| Target | Forward primer | Reverse Primer | Probe |
|---|---|---|---|
| AnD9Ds | SEQ ID NO: 31 | SEQ ID NO: 32 | UPL #9 |
| | GGACTTCTCTACTCTCACCTTGGA | TCCGATCCTCTTTGGGTTCT | |
| HzD9Ds | SEQ ID NO: 33 | SEQ ID NO: 34 | UPL #143 |
| | GACCCACACAATGCAACG | CCTAACAAGAAGCCAGCCAAT | |
| LnD9Ds | SEQ ID NO: 35 | SEQ ID NO: 36 | UPL #7 |
| | GTTCTGACTGCGTTGGTCAC | CGGAAACTCATGGTGGAAGT | |
| Actin | SEQ ID NO: 37 | SEQ ID NO: 38 | SEQ ID NO: 39 |
| | CTACTGGTATTGTGCTCGACT | CTCTCTCGGTGAGAATCTTCAT | CACGCTATCCTCCGTCTCGATC |

| Target | Label |
|---|---|
| AnD9Ds | FAM |
| HzD9Ds | FAM |
| LnD9Ds | FAM |
| Actin | Tx-Red |

RT-PCR reactions were run on a LightCycler 480II real-time PCR thermal cycler (Roche). Data for target UPL assays was collected using a 533 nm emission filter and a 483 nm excitation signal. Data for the actin reference assay was collected using a 610 nm filter and a 558 nm excitation signal. Cycle time values and target to reference ratios were calculated automatically using the LC480II software's “Advanced Relative Quantification” analysis workflow. Relative accumulation of desaturase transcript levels within each sample was calculated using the standard ΔΔCt method (Roche).

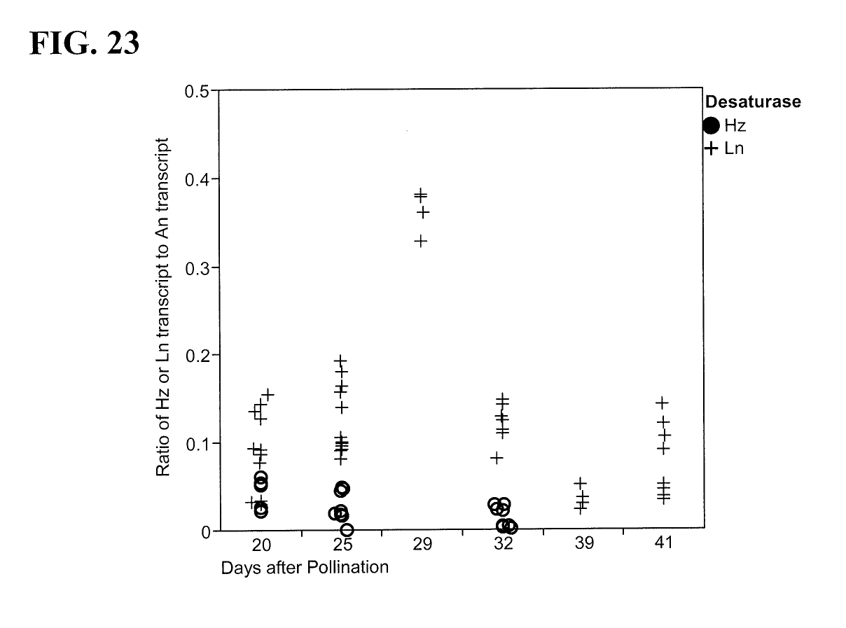

For each canola seed sample from pDAB7319 (AnD9DS v3 and LnD9DS-2 v2) and pDAB7324 (AnD9DS v3 and HzD9DS v2), transcript accumulation of HzD9DS or LnD9DS-2 transgenes was significantly lower than the transcript of AnD9DS in the same events. The observed differences in transcript accumulation varied between 3- and 20-fold less. FIG. 23. Thus, insufficient expression of HzD9DS and LnD9DS-2 may account for the lack of detection of the polypeptide and absence of phenotype attributable to these genes.

### Example 13: Expression of the Delta-9 Desaturase PTUs by Alternative Promoters

The use of additional transcriptional regulatory regions to express gene(s) encoding LnD9DS-2, HzD9DS, and MgD9DS proteins can further increase the content of these delta-9 desaturases within canola. Identification and use of transcriptional regulatory regions which express earlier in development, and for longer periods of time, can increase the levels of heterologous delta-9 desaturases within canola seed by promoting robust seed-specific transcription of a heterologous gene at earlier stages of seed development. Examples of such transcriptional regulatory regions include, but are not limited to, the LfKCS3 promoter (U.S. Pat. No. 7,253,337) and FAE 1 promoter (U.S. Pat. No. 6,784,342). These promoters are used singularly, or in combination, to drive the expression of LnD9DS-2, HzD9DS, and MgD9DS expression cassettes, for example, through operable linkage with genes such as those previously described in plasmids, pDAB7319; pDAB7321; pDAB7324; pDAB7326; pDAB7328; and pDAB7330. Methods to replace transcriptional regulatory regions within a plasmid are well-known within the art. As such, a polynucleotide fragment comprising the PvPhas promoter is removed from pDAB7319, pDAB7321, pDAB7324, pDAB7326, pDAB7328, or pDAB7330 (or the preceding plasmids used to build pDAB7319, pDAB7321, pDAB7324, pDAB7326, pDAB7328, or pDAB7330), and replaced with either a LfKCS3 or FAE 1 promoter region. The newly-constructed plasmids are used to stably transform canola plants, according to the procedures set forth in the previous examples. Transgenic canola plants are isolated and molecularly characterized. The resulting delta-9 desaturase accumulation is determined, and canola plants which robustly express delta-9 desaturase are identified.

Further modifications to the transcriptional regulatory regions for increased expression of a delta-9 desaturase include replacing the existing Kozak sequence with any of the sequences described in Table 17. The engineering of alternative Kozak sequences upstream of the start site of a delta-9 desaturase is completed using standard molecular biology techniques. Synthetic polynucleotide fragments are synthesized and cloned upstream of a delta-9 desaturase coding sequence using techniques known within the art. The context of the start codon has a strong effect on the level of expression of a transgene. Modifying the Kozak sequence to one listed in Table 17 increases the levels of expression of the heterologous delta-9 desaturase.

TABLE 17
Kozak sequences which are incorporated upstream of
a heterologous delta-9 desaturase gene to increase
expression.

| Kozak Sequence | SEQ ID NO: | Sequence |
|---|---|---|
| Kozak #1 | SEQ ID NO: 40 | GGATCCAACAATG |
| Kozak #2 | SEQ ID NO: 41 | ACAACCAAAAATG |
| Kozak #3 | SEQ ID NO: 42 | ACAACCAACCTACCATGG |
| Kozak #4 | SEQ ID NO: 43 | ACAACCAAAAAATG |

### Example 14: Design and Synthesis of Delta-9 Desaturase Genes from Helicoverpa zea and Leptosphaeria nodorum

To obtain higher levels of expression of heterologous genes in plants, the codon optimization strategy described in Example 2 was modified, and the heterologous gene protein coding regions for HzD9DS and LnD9DS-2 were re-engineered using a new design protocol.

Codon selection was made using a table which had calculated the codon bias of the prospective host plant, which in this case was canola. In designing coding regions for plant expression of delta-9 desaturase genes, the primary (“first choice”) codons preferred by the plant were determined, and used at about 95% of the time. “Second choice” codons were used sparingly, at a frequency of about 5%. Accordingly, a new DNA sequence was designed which encodes the amino sequence of each delta-9 desaturase, wherein the new DNA sequence differed from the native delta-9 desaturase gene by the substitution of plant first preferred and second preferred codons to specify an appropriate amino acid at each position within the amino acid sequence. The new sequence was then analyzed for restriction enzyme sites that might have been created by the modifications. The identified restriction enzyme sites were then removed by replacing the codons with first or second choice preferred codons. Other sites in the sequence which could affect transcription or translation of the gene of interest, specifically highly stable stem loop structures, were also removed.

The selections of preferred codon choices (first and second choices) from the genetic code of canola were determined from a codon bias table compiled from the protein coding sequences for canola. In Tables 18 and 19, Columns labeled as “Native Gene %” present the distributions (in % of usage for all codons for that amino acid) of synonymous codons for each amino acid, as found in the coding regions of Brassica napus (canola). New DNA sequences which encode essentially the amino acid sequence of the M. grisea, H. zea and L. nodorum delta-9 desaturases were designed for optimal expression in canola using the preferred codon distribution of first and second choice codons found in canola genes. Design of the plant-optimized DNA sequences were initiated by reverse-translation of the protein sequences of SEQ ID NO:12 (M. grisea), SEQ ID NO:13 (H. zea), and SEQ ID NO:14 (L. nodorum) using the canola codon bias table constructed. Columns labeled as “Plnt Opt Gene %” indicate the preferred codons and the frequency with which they were incorporated into the delta-9 desaturase gene design. SEQ ID NO:44 and SEQ ID NO:45 set forth the nucleotide sequences of the new canola-optimized LnD9DS-2 and HzD9DS desaturases, respectively. These new canola-optimized sequences were labeled as LnD9DS-2 v3 and HzD9DS v3.

TABLE 18
Codon compositions of coding regions for the HzD9DS protein. The native H. zea
desaturase coding region is compared to a Plant-Optimized version.

| | | | | | | | | | | | | | Pint |
|---|---|---|---|---|---|---|---|---|---|---|---|---|---|
| Amino | | Native | Native | Pint Opt | Pint Opt | Pint Opt | Amino | | Native | Native | Pint Opt | Pint Opt | Opt |
| Acid | Codon | Gene # | Gene % | Gene # | Gene % | Recm'd | Acid | Codon | Gene # | Gene % | Gene # | Gene % | Recm'd |
| ALA (A) | GCA | 4 | 11.4 | 1 | 2.9 | 0.0 | LEU (L) | CTA | 2 | 5.9 | 0 | 0.0 | 0.0 |
| | GCC | 7 | 20.0 | 0 | 0.0 | 0.0 | | CTC | 8 | 23.5 | 0 | 0.0 | 0.0 |
| | GCG | 8 | 22.9 | 0 | 0.0 | 0.0 | | CTG | 14 | 41.2 | 0 | 0.0 | 0.0 |
| | GCT | 16 | 45.7 | 34 | 97.1 | 100.0 | | CTT | 6 | 17.6 | 34 | 100.0 | 100.0 |
| ARG (R) | AGA | 1 | 7.7 | 0 | 0.0 | 0.0 | | TTA | 2 | 5.9 | 0 | 0.0 | 0.0 |
| | AGG | 5 | 38.5 | 13 | 100.0 | 100.0 | | TTG | 2 | 5.9 | 0 | 0.0 | 0.0 |
| | CGA | 2 | 15.4 | 0 | 0.0 | 0.0 | LYS (K) | AAA | 11 | 44.0 | 0 | 0.0 | 0.0 |
| | CGC | 5 | 38.5 | 0 | 0.0 | 0.0 | | AAG | 14 | 56.0 | 25 | 100.0 | 100.0 |
| | CGG | 0 | 0.0 | 0 | 0.0 | 0.0 | MET (M) | ATG | 8 | 100 | 8 | 100 | 100.0 |
| | CGT | 0 | 0.0 | 0 | 0.0 | 0.0 | PHE (F) | TTC | 20 | 83.3 | 24 | 100.0 | 100.0 |
| ASN (N) | AAC | 13 | 72.2 | 18 | 100.0 | 100.0 | | TTT | 4 | 16.7 | 0 | 0.0 | 0.0 |
| | AAT | 5 | 27.8 | 0 | 0.0 | 0.0 | PRO (P) | CCA | 1 | 6.3 | 16 | 100.0 | 100.0 |
| ASP (D) | GAC | 16 | 64.0 | 2 | 8.0 | 0.0 | | CCC | 5 | 31.3 | 0 | 0.0 | 0.0 |
| | GAT | 9 | 36.0 | 23 | 92.0 | 100.0 | | CCG | 2 | 12.5 | 0 | 0.0 | 0.0 |
| CYS (C) | TGC | 1 | 100.0 | 1 | 100.0 | 100.0 | | CCT | 8 | 50.0 | 0 | 0.0 | 0.0 |
| | TGT | 0 | 0.0 | 0 | 0.0 | 0.0 | SER (S) | AGC | 2 | 12.5 | 0 | 0.0 | 0.0 |
| END | TAA | 1 | 100.0 | 0 | 0.0 | 0.0 | | AGT | 1 | 6.3 | 0 | 0.0 | 0.0 |
| | TAG | 0 | 0.0 | 0 | 0.0 | 0.0 | | TCA | 1 | 6.3 | 1 | 6.3 | 0.0 |
| | TGA | 0 | 0.0 | 1 | 100.0 | 100.0 | | TCC | 6 | 37.5 | 0 | 0.0 | 0.0 |
| GLN (Q) | CAA | 2 | 33.3 | 6 | 100.0 | 100.0 | | TCG | 3 | 18.8 | 0 | 0.0 | 0.0 |
| | CAG | 4 | 66.7 | 0 | 0.0 | 0.0 | | TCT | 3 | 18.8 | 15 | 93.8 | 100.0 |
| GLU (E) | GAA | 7 | 63.6 | 0 | 0.0 | 0.0 | THR (T) | ACA | 3 | 16.7 | 0 | 0.0 | 0.0 |
| 16 | GAG | 4 | 36.4 | 11 | 100.0 | 100.0 | | ACC | 7 | 38.9 | 18 | 100.0 | 100.0 |
| GLY (G) | GGA | 8 | 40.0 | 20 | 100.0 | 100.0 | | ACG | 4 | 22.2 | 0 | 0.0 | 0.0 |
| | GGC | 6 | 30.0 | 0 | 0.0 | 0.0 | | ACT | 4 | 22.2 | 0 | 0.0 | 0.0 |
| | GGG | 2 | 10.0 | 0 | 0.0 | 0.0 | TRP (W) | TGG | 14 | 100 | 14 | 100 | 0.0 |
| | GGT | 4 | 20.0 | 0 | 0.0 | 0.0 | TYR (Y) | TAC | 12 | 80.0 | 15 | 100.0 | 100.0 |
| HIS (H) | CAC | 11 | 73.3 | 15 | 100.0 | 100.0 | | TAT | 3 | 20.0 | 0 | 0.0 | 0.0 |
| | CAT | 4 | 26.7 | 0 | 0.0 | 0.0 | VAL (V) | GTA | 0 | 0.0 | 0 | 0.0 | 0.0 |
| ILE (I) | ATA | 3 | 15.0 | 1 | 5.0 | 0.0 | | GTC | 5 | 26.3 | 0 | 0.0 | 0.0 |
| | ATC | 10 | 50.0 | 19 | 95.0 | 100.0 | | GTG | 13 | 68.4 | 0 | 0.0 | 0.0 |
| | ATT | 7 | 35.0 | 0 | 0.0 | 0.0 | | GTT | 1 | 5.3 | 19 | 100.0 | 100.0 |
| | Totals | 165 | | 165 | | | | Totals | 189 | | 189 | | |

TABLE 19
Codon compositions of coding regions for the LnD9DS-2 protein. The native L. nodorum
desaturase coding region is compared to a Plant-Optimized version.

| | | | | | | | | | | | | | Pint |
|---|---|---|---|---|---|---|---|---|---|---|---|---|---|
| Amino | | Native | Native | Pint Opt | Pint Opt | Pint Opt | Amino | | Native | Native | Pint Opt | Pint Opt | Opt |
| Acid | Codon | Gene # | Gene % | Gene # | Gene % | Recm'd | Acid | Codon | Gene # | Gene % | Gene # | Gene % | Recm'd |
| ALA (A) | GCA | 3 | 9.4 | 0 | 0.0 | 0.0 | LEU (L) | CTA | 7 | 15.6 | 0 | 0.0 | 0.0 |
| | GCC | 9 | 28.1 | 0 | 0.0 | 0.0 | | CTC | 14 | 31.1 | 0 | 0.0 | 0.0 |
| | GCG | 12 | 37.5 | 0 | 0.0 | 0.0 | | CTG | 7 | 15.6 | 0 | 0.0 | 0.0 |
| | GCT | 8 | 25.0 | 32 | 100.0 | 100.0 | | CTT | 5 | 11.1 | 45 | 100.0 | 100.0 |
| ARG (R) | AGA | 4 | 13.8 | 1 | 3.4 | 0.0 | | TTA | 3 | 6.7 | 0 | 0.0 | 0.0 |
| | AGG | 3 | 10.3 | 28 | 96.6 | 100.0 | | TTG | 9 | 20.0 | 0 | 0.0 | 0.0 |
| | CGA | 7 | 24.1 | 0 | 0.0 | 0.0 | LYS (K) | AAA | 9 | 45.0 | 0 | 0.0 | 0.0 |
| | CGC | 8 | 27.6 | 0 | 0.0 | 0.0 | | AAG | 11 | 55.0 | 20 | 100.0 | 100.0 |
| | CGG | 5 | 17.2 | 0 | 0.0 | 0.0 | MET (M) | ATG | 9 | 100 | 9 | 100 | 100.0 |
| | CGT | 2 | 6.9 | 0 | 0.0 | 0.0 | PHE (F) | TTC | 16 | 80.0 | 20 | 100.0 | 100.0 |
| ASN (N) | AAC | 6 | 50.0 | 12 | 100.0 | 100.0 | | TTT | 4 | 20.0 | 0 | 0.0 | 0.0 |
| | AAT | 6 | 50.0 | 0 | 0.0 | 0.0 | PRO (P) | CCA | 3 | 16.7 | 18 | 100.0 | 100.0 |
| ASP (D) | GAC | 16 | 66.7 | 2 | 8.3 | 0.0 | | CCC | 8 | 44.4 | 0 | 0.0 | 0.0 |
| | GAT | 8 | 33.3 | 22 | 91.7 | 100.0 | | CCG | 2 | 11.1 | 0 | 0.0 | 0.0 |
| CYS (C) | TGC | 4 | 80.0 | 5 | 100.0 | 100.0 | | CCT | 5 | 27.8 | 0 | 0.0 | 0.0 |
| | TGT | 1 | 20.0 | 0 | 0.0 | 0.0 | SER (S) | AGC | 8 | 27.6 | 0 | 0.0 | 0.0 |
| END | TAA | 0 | 0.0 | 0 | 0.0 | 0.0 | | AGT | 6 | 20.7 | 0 | 0.0 | 0.0 |
| | TAG | 1 | 100.0 | 0 | 0.0 | 0.0 | | TCA | 1 | 3.4 | 1 | 3.4 | 0.0 |
| | TGA | 0 | 0.0 | 1 | 100.0 | 100.0 | | TCC | 6 | 20.7 | 0 | 0.0 | 0.0 |
| GLN (Q) | CAA | 10 | 55.6 | 18 | 100.0 | 100.0 | | TCG | 7 | 24.1 | 0 | 0.0 | 0.0 |
| | CAG | 8 | 44.4 | 0 | 0.0 | 0.0 | | TCT | 1 | 3.4 | 28 | 96.6 | 100.0 |
| GLU (E) | GAA | 5 | 33.3 | 1 | 6.7 | 0.0 | THR (T) | ACA | 11 | 44.0 | 0 | 0.0 | 0.0 |
| 16 | GAG | 10 | 66.7 | 14 | 93.3 | 100.0 | | ACC | 5 | 20.0 | 25 | 100.0 | 100.0 |
| GLY (G) | GGA | 13 | 34.2 | 38 | 100.0 | 100.0 | | ACG | 7 | 28.0 | 0 | 0.0 | 0.0 |
| | GGC | 16 | 42.1 | 0 | 0.0 | 0.0 | | ACT | 2 | 8.0 | 0 | 0.0 | 0.0 |
| | GGG | 6 | 15.8 | 0 | 0.0 | 0.0 | TRP (W) | TGG | 19 | 100 | 19 | 100 | 0.0 |
| | GGT | 3 | 7.9 | 0 | 0.0 | 0.0 | TYR (Y) | TAC | 11 | 64.7 | 17 | 100.0 | 100.0 |
| HIS (H) | CAC | 12 | 66.7 | 18 | 100.0 | 100.0 | | TAT | 6 | 35.3 | 0 | 0.0 | 0.0 |
| | CAT | 6 | 33.3 | 0 | 0.0 | 0.0 | VAL (V) | GTA | 6 | 17.6 | 0 | 0.0 | 0.0 |
| ILE (I) | ATA | 4 | 18.2 | 1 | 4.5 | 0.0 | | GTC | 10 | 29.4 | 0 | 0.0 | 0.0 |
| | ATC | 9 | 40.9 | 21 | 95.5 | 100.0 | | GTG | 12 | 35.3 | 0 | 0.0 | 0.0 |
| | ATT | 9 | 40.9 | 0 | 0.0 | 0.0 | | GTT | 6 | 17.6 | 34 | 100.0 | 100.0 |
| | Totals | 214 | | 214 | | | | Totals | 236 | | 236 | | |

Syntheses of DNA fragments comprising SEQ ID NO:44 and SEQ ID NO:45 were performed by PicoScript and Blue Heron Biotechnology. The synthetic DNA was then cloned into expression vectors and transformed into canola substantially as described in the foregoing examples.

### Example 15: Modification of N- and C-Termini to Increase the Accumulation of Acyl-CoA Desaturase Polypeptides in Plants

The accumulation and stability of membrane-bound proteins in the endoplasmic reticulum (ER) can be influenced by amino acid sequence motifs and modifications at their N- and C-termini. Ravid and Hochstrasser (2008) Nat. Rev. Mol. Cell. Biol. 9:679-90. In particular, N- and C-terminal motifs and modifications have been shown to modulate the accumulation and stability of lipid desaturases in fungi and plants, as well as animals. McCartney et al. (2004) Plant J. 37:156-73; Mziaut et al. (2000) Proc. Natl. Acad. Sci. U.S.A. 97:8883-8.

The addition of either a Myc or the hemagglutin (HA) epitope tag to the N-terminal of FAD2 or FAD3 significantly increases the steady state level of these enzymes within yeast. O'Quin et al. (2009) Appl Microbiol Biotechnol 83:117-25. Accordingly, the addition of these, or similar epitopes, to the N-terminus of a delta-9 desaturase of the present invention is utilized to increase the expression of the polypeptide in a plant. A polynucleotide linker that encodes a Myc tag (SEQ ID NO:46) or a HA tag (SEQ ID NO:47) is cloned within the 5′ end of a delta-9 desaturase (e.g., HzD9DS, MgD9DS, AnD9DS, LnD9DS-1, and LnD9DS-2) coding sequence as a contiguous open reading frame. The resulting coding sequence is cloned within a plant expression plasmid using the cloning strategy described in Example 3. The newly-constructed plasmid is used to stably transform an Arabidopsis and/or canola plant cell, material, or tissue. Transgenic plants are regenerated from the transformed plant cell, material, or tissue. Transgenic plants are isolated and molecularly characterized. The resulting delta-9 desaturase accumulation in seeds of transgenic plants is determined, and plants which robustly express the delta-9 desaturase polypeptide are identified.

Evidence from expression of AnD9DS in Arabidopsis and canola (Examples 11 and 12) indicates a significantly higher level of expression of this particular desaturase enzyme, relative to HzD9DS and LnD9DS-2. Thus, all or parts of the N- and C-termini lying outside the core desaturase domain (containing the transmembrane segments and conserved catalytic histidine residues) of AnD9DS may be used to replace equivalent residues in the lower-expressing desaturases and increase expression thereof. Accordingly, all or part of N-terminal residues 1-68 and C-terminal residues 281-455 of AnD9DS (SEQ ID NO:72 and SEQ ID NO:73, respectively) are used to replace all or part of the 68 N-terminal residues (1-68) and 168 C-terminal residues (281-449) of LnD9DS-2 (SEQ ID NO:14) and/or the 76 N-terminal residues (1-76) and 60 C-terminal residues (293-353) of HzD9DS (SEQ ID NO:1.3). The resulting coding sequence is cloned within a plant expression plasmid using the cloning strategy described in Example 3. The newly-constructed plasmid is used to stably transform an Arabidopsis and/or canola plant cell, material, or tissue. Transgenic plants are regenerated from the transformed plant cell, material, or tissue. Transgenic plants are isolated and molecularly characterized. The resulting delta-9 desaturase accumulation in seeds of transgenic plants is determined, and plants which robustly express the modified HzD9DS or modified LnD9DS-2 polypeptide are identified.

### Example 16: Modifications to Enhance mRNA Expression of Acyl-CoA Desaturase within Plants

It is known within the art that the expression of mRNA can be enhanced by the incorporation of genetic elements that stabilize and increase mRNA accumulation. The incorporation of 5′ and 3′ untranslated regions (e.g., Tobacco Osmotin 5′ and 3′ UTR sequences (Liu et al. (2003) Nat. Biotechnol. 21:1222-8), and the Tobacco Mosaic Virus Ω sequence (Gallie et al. (1987) Nucleic Acids Res. 15:8693-711)) or introns (Koziel et al. (1996) Plant Mol. Biol. 32:393-405), within close proximity of a HzD9Ds or LnD9DS-2 coding sequence, is used to increase the levels of expression of the transgene when compared to the expression of the same coding sequence lacking the aforementioned genetic elements. The addition one or more of these genetic elements within a desaturases PTU is performed according to methods well-known in the art. Polynucleotide fragments comprising the 5′ untranslated region, 3′ untranslated region, and/or intron are added to a plant expression plasmid (e.g., pDAB7319, pDAB7321, pDAB7324, pDAB7326, pDAB7328, pDAB7330, or the preceding plasmids used to build pDAB7319, pDAB7321, pDAB7324, pDAB7326, pDAB7328, or pDAB7330) via standard cloning methods. The newly-constructed plasmid is used to stably transform an Arabidopsis and/or canola plant cell, material, or tissue. Transgenic plants are regenerated from the transformed plant cell, material, or tissue. Transgenic plants are isolated and molecularly characterized. The resulting delta-9 desaturase accumulation in seeds of transgenic plants is determined, and plants which robustly express the HzD9DS or LnD9DS-2 polypeptide are identified.

Furthermore, it is know in the art that yeast desaturase genes such as OLE1 are highly-regulated. The deletion of sequences that encode transmembrane regions and that are a part of the cytochrome b5 domain reduce the stability of the OLE1 transcript. Vemula et al. (2003) J. Biol. Chem. 278(46):45269-79. The presence of these sequences within OLE1 act as mRNA stabilizing sequences. Accordingly, incorporation of the OLE1 sequences that encode the transmembrane region and cytochrome b5 domain into a LnD9DS-2 or HzD9DS coding sequence is utilized to increase stability of the mRNA transcript of the coding sequence, thereby resulting in higher levels of expression and a subsequent increase of LnD9DS-2 or HzD9DS polypeptide. A chimeric LnD9DS-2 or HzD9DS coding sequence that includes the OLE1 transmembrane region and cytochrome b5 domain sequences is constructed using methods known in the art. The coding sequence produced thereby is incorporated into a plant expression plasmid (e.g., as described in the foregoing examples), and used to generate transgenic plants via Agrobacterium-mediated plant transformation. Transgenic plants are isolated and characterized. The resulting delta-9 desaturase accumulation is determined, and plants which robustly express the delta-9 desaturase are identified.

### Example 17

### Use of an Alternative 3′ Untranslated Region Terminator for Stable Expression of a Delta-9 Desaturase in a Plant

Due to a limited number of available 3′ UTR-terminators, the Agrobacterium ORF 23 3′ UTR-terminator (AtuORF23 3′ UTR) is typically used to terminate transcription. It was recently shown that other 3′ UTR-terminators are more effective in terminating transcriptional read-through in Arabidopsis thaliana. Accordingly, the Phaseolus vulgaris Phaseolin 3′UTR-teminator (SEQ ID NO:69) is used in combination with the Phaseolus vulgaris Phaseolin promoter to reduce transcriptional read-through of upstream genes, thereby reducing transcriptional interference.

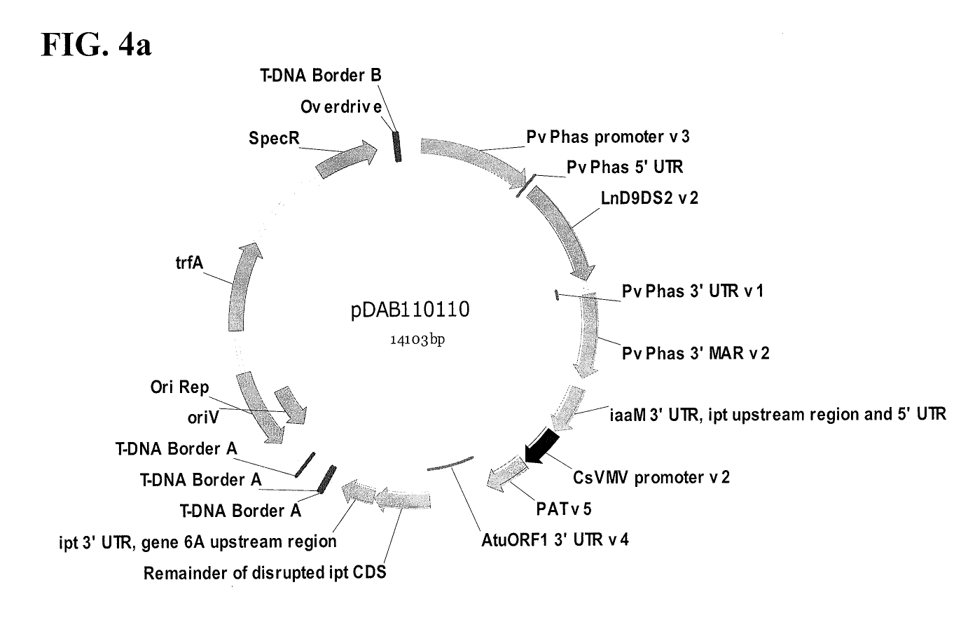

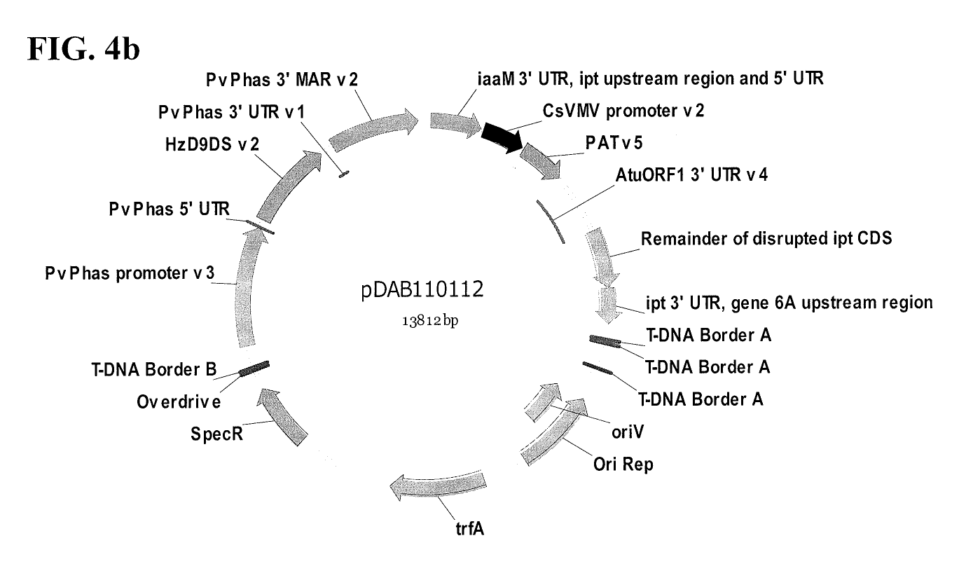

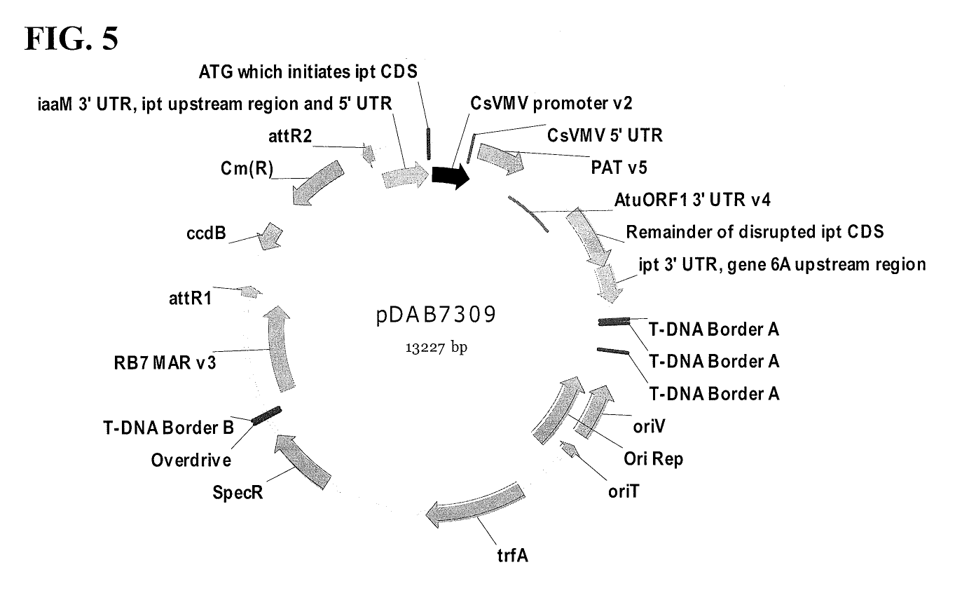

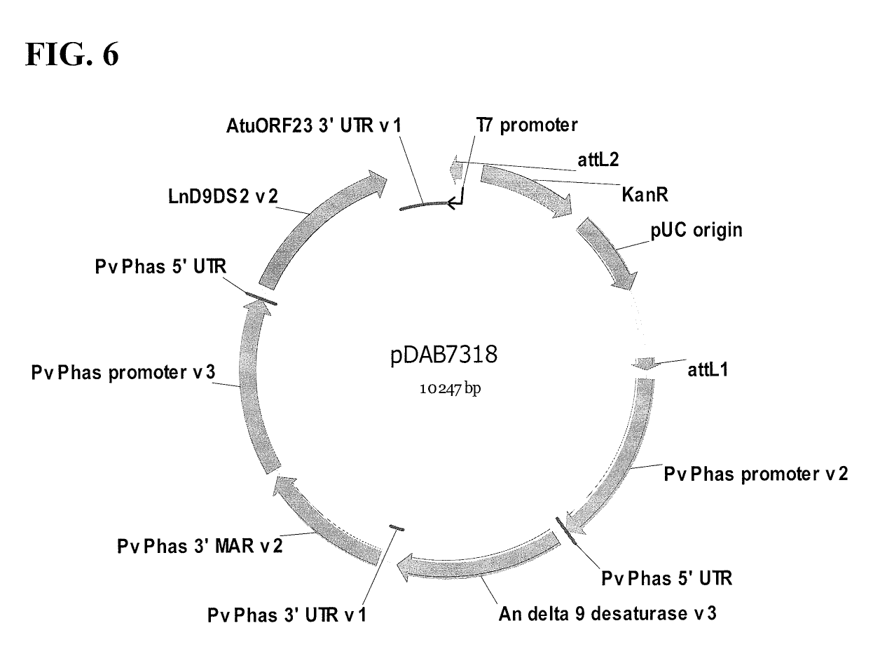

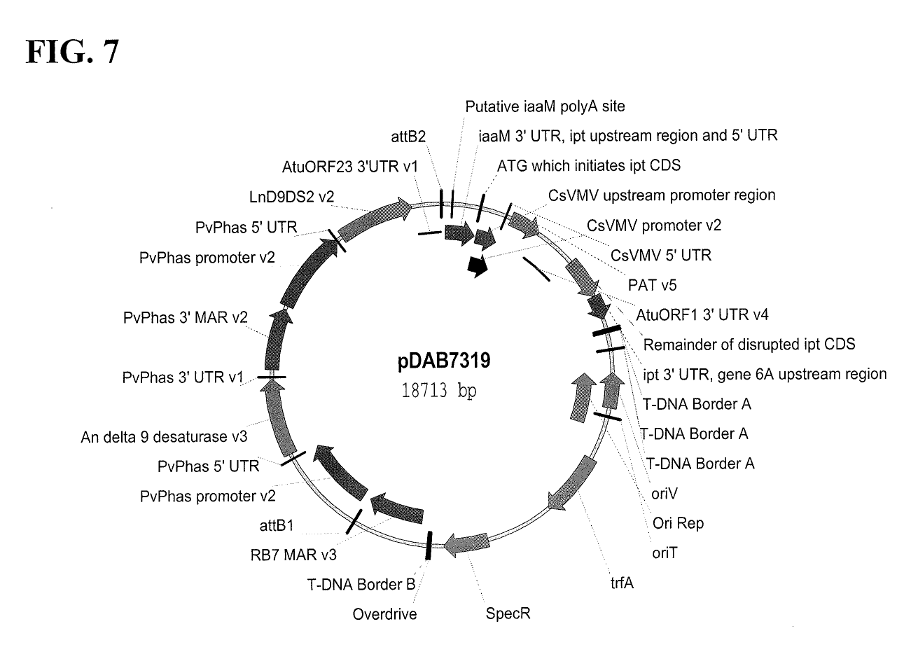

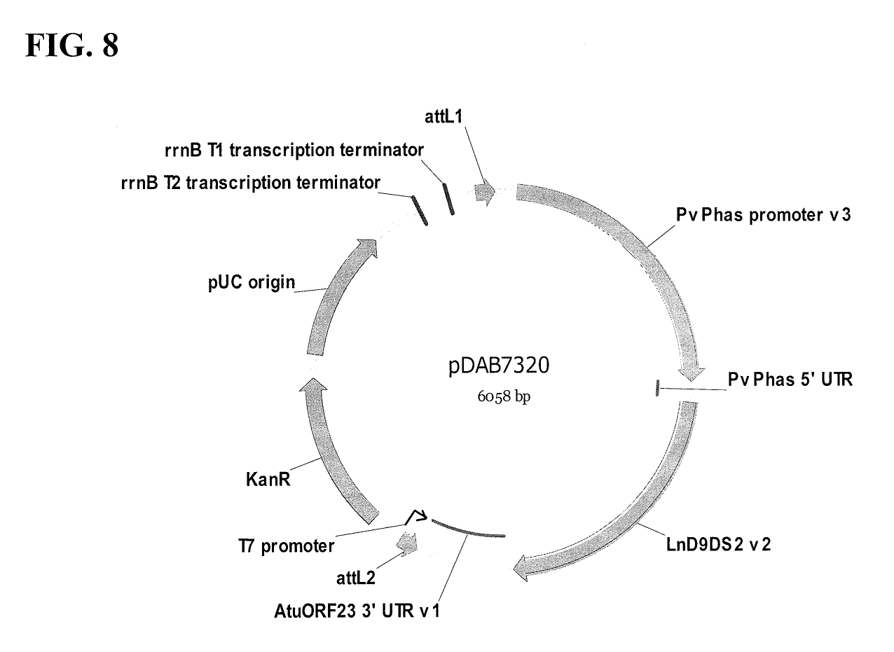

The Phaseolus vulgaris Phaseolin 3′UTR-terminator (PvPhas 3′UTR v1) was incorporated within an LnD9DS-2 v2 expression cassette, and within an HzD9DS v2 expression cassette, which were previously described in plasmid pDAB7321 and pDAB7326. According to methods well-known to those of skill in the art, a polynucleotide fragment comprising the PvPhas 3′UTR v1 was placed downstream of a LnD9DS-2 v2 gene to create binary plasmid, pDAB110110 (FIG. 4a; SEQ ID NO:74). A polynucleotide fragment comprising the PvPhas 3′UTR v1 was also placed downstream of a HzD9DS v2 gene to create binary plasmid pDAB110112 (FIG. 4b; SEQ ID NO:75).

The resulting binary plasmids were confirmed via restriction enzyme digestion and sequencing. The newly-constructed plasmids are each used to stably transform an Arabidopsis and/or canola plant cell, material, or tissue. Transgenic plants are regenerated from the transformed plant cell, material, or tissue. Transgenic plants are isolated and molecularly characterized. The resulting delta-9 desaturase accumulation in seeds of the transgenic plants is determined, and plants which robustly express the HzD9DS or LnD9DS-2 polypeptide are identified.

What is claimed is:

1. An isolated nucleic acid molecule encoding a delta-9 desaturase enzyme comprising an amino acid sequence being at least 80% identical to a sequence selected from the group consisting of SEQ ID NO:12, SEQ ID NO:13, SEQ ID NO:14, SEQ ID NO:50, SEQ ID NO:51, SEQ ID NO:52, SEQ ID NO:72, and SEQ ID NO:73.

2. The nucleic acid molecule of claim 1, wherein the nucleic acid molecule comprises a nucleotide sequence at least 60% identical to a sequence selected from the group consisting of SEQ ID NO:3, SEQ ID NO:4, SEQ ID NO:5, SEQ ID NO:8, SEQ ID NO:9, SEQ ID NO:10, SEQ ID NO:11, SEQ ID NO:15, SEQ ID NO:16, SEQ ID NO:17, SEQ ID NO:44, and SEQ ID NO:45.

3. The nucleic acid molecule of claim 1, further comprising a gene regulatory element.

4. The nucleic acid molecule of claim 3, wherein the gene regulatory element is selected from the group consisting of the Saccharomyces cerevisiae delta-9 desaturase promoter, the delta-9 desaturase 3′UTR/terminator, the ole1 gene promoter, the phaseolin promoter, the Phaseolus vulgaris phaseolin 5′ untranslated region, the Phaseolus vulgaris phaseolin 3′ untranslated region, the Phaseolus vulgaris phaseolin matrix attachment region, the Agrobacterium tumefaciens ORF23 3′ untranslated region, the Cassava vein Mosaic Virus Promoter, the Agrobacterium tumefaciens ORF1 3′ untranslated region, the Nicotiana tabacum RB7 Matrix Attachment Region, Overdrive, T-stand border sequences, the LfKCS3 promoter, FAE 1 promoter, SEQ ID NO:40, SEQ ID NO:41, SEQ ID NO:42, SEQ ID NO:43, a Myc tag, and a hemagglutin tag.

5. An isolated delta-9 desaturase enzyme comprising an amino acid sequence at least 80% identical to a sequence selected from the group consisting of SEQ ID NO:12, SEQ ID NO:13, SEQ ID NO:14, SEQ ID NO:50, SEQ ID NO:51, SEQ ID NO:52, SEQ ID NO:72, and SEQ ID NO:73.

6. A chimeric delta-9 desaturase polypeptide comprising SEQ ID NO:72 and/or SEQ ID NO:73, wherein the polypeptide further comprises an amino acid sequence selected from the group consisting of SEQ ID NO:77 and SEQ ID NO:78.

7. A method for decreasing the amount of saturated fatty acids in a cell, the method comprising:
transforming a cell with at least one nucleic acid molecule of claim 1, such that the amount of saturated fatty acids in the cell is decreased.

8. The method according to claim 7, wherein the cell is a yeast cell.

9. The method according to claim 7, wherein the cell is a plant cell.

10. The method according to claim 9, wherein transforming the plant cell introduces into the plant cell a means for decreasing levels of 16:0-ACP in the plant cell.

11. The method according to claim 10, wherein the means for decreasing levels of 16:0-ACP in the plant cell is an extraplastidial desaturase.

12. The method of claim 11, wherein the extraplastidial desaturase is a desaturase selected from the group consisting of LnD9DS desaturase, AnD9DS desaturase, HzD9DS desaturase, and MgD9DS desaturase.

13. The method according to claim 9, wherein the plant cell is obtained from a plant selected from a genus selected from the group consisting of Arabidopsis, Borago, Canola, Ricinus, Theobroma, Zea, Gossypium, Crambe, Cuphea, Linum, Lesquerella, Limnanthes, Linola, Tropaeolum, Oenothera, Olea, Elaeis, Arachis, rapeseed, Carthamus, Glycine, Soja, Helianthus, Nicotiana, Vernonia, Triticum, Hordeum, Oryza, Avena, Sorghum, Secale, and the other members of the Gramineae.

14. An oil seed plant comprising the nucleic acid sequence of claim 1.

15. A plant seed which expresses an extraplastidial desaturase selected from the group consisting of NO:12, SEQ ID NO:13, SEQ ID NO:14, SEQ ID NO:50, SEQ ID NO:51, and SEQ ID NO:52.

16. A seed of a transgenic Brassica napus line, the seed having a decreased levels of 16:0, relative to an isogenic version of the transgenic Brassica napus line.

17. A method for creating a genetically engineered plant comprising decreased amounts of saturated fatty acids in the plant compared to the wild type plant, the method comprising:
transforming plant material with the nucleic acid molecule of claim 1; and
culturing the transformed plant material to obtain a plant.

18. The method of claim 17, wherein the plant is selected from a genus selected from the group consisting of Arabidopsis, Borago, Canola, Ricinus, Theobroma, Zea, Gossypium, Crambe, Cuphea, Linum, Lesquerella, Limnanthes, Linola, Tropaeolum, Oenothera, Olea, Elaeis, Arachis, rapeseed, Carthamus, Glycine, Soja, Helianthus, Nicotiana, Vernonia, Triticum, Hordeum, Oryza, Avena, Sorghum, Secale, and the other members of the Gramineae.

19. A plant obtained by the method of claim 17.

20. A plant material obtained from the plant of claim 19.

21. The plant material of claim 20, wherein the plant material is a seed.


SEQUENCE LISTING (78 sequences)

SEQ ID NO: 1 - DNA, 44 bp, Artificial
  Forward primer Mgdelta9F
gaagaattcatggcttcgtcatcttcctccgtgccggagttggc

SEQ ID NO: 2 - DNA, 41 bp, Artificial
  Reverse primer Mg9deltaR
ctcgagctagttagtcacgcggcctgagcacccggaacagg

SEQ ID NO: 3 - DNA, 1523 bp, Artificial
  PCR amplified fragment of MgD9Ds
gaattcatggcttcgtcatcttcctccgtgccggagttggctgccgccttccctgatggcactaccgacttcaagcccatgaggaacaccaagggctacgacgtcagcaagccgcacatttccgagacacctatgacactcaagaactggcataagcacgtcaactggctcaacaccaccttcatcttgtttgtgcccctggctggtctcatatccacttactgggtccctctgcagtggaagacggctgtatgggctgtcgtctactacttcaacaccggcctgggaattactgccggtaagtggctcttgaacaaacgagctaggccgccgccctgtatccaatcatctgtatccatccctagatgctaactagaaaacttgcgggttaccaccgactttgggctcacagctcgtacaaggcctcgcttccgctcaaaatctaccttgccgccgttggcgctggtgccgtcgagggctccatcagatggtggtccaacggtcaccgcgcacaccaccgatacaccgataccgagaaggacccctactcagtccgcaagggtctcctgtactcacacatgggatggatgcttctgaagcagaaccccaagaagcagggccgcaccgacatcaccgacctgaacgaggaccccgttgtcgtttggcagcaccgcaacttcctcaagtgtgttatcttcatggccctcgtcttccccacacttgtggctggccttggctggggtgactactggggaggtttcatctacggaggtattctgcgtgtcttcttcgtccagcaggccaccttctgcgtcaactcgcttgcccactggctcggtgaccagcctttcgacgatcgcaactcgccgcgtgatcacgtcatcacagccctggtcacccttggagagggataccacaacttccaccacgagttcccttcggactaccgcaacgctattgagtggtaccagtatgaccccaccaagtggtcaatctggatctggaagcagcttggtcttgcccacaacctgaagcagttccgccaaaacgagattgagaagggacgcgtccagcagctgcagaagaagctcgaccagaagcgcgccaagcttgattggggtattcccttggagcagcttcccgttgttagctgggatgactttgttgagcagtccaagaacggaaaggcttggattgcagttgccggtgtcatccacgatgttggtgacttcatcaaggaccaccctggtggcagagctctcatcaactcggccattggcaaggacgcaaccgcaatcttcaacggcggtgtttacaaccactccaacgccgctcacaacctgctctcgactatgcgtgtgggtgttttgcgtggcggctgcgaggttgagatctggaagcgcgcccagtccgaaaacaaggacgtctcaaccgtcgttgattcttcgggtaaccgcatcgtccgcgcgggtgggcaagcgaccaaggtcgtccagcctgttccgggtgctcaggccgcgtga

SEQ ID NO: 4 - DNA, 1428 bp, Artificial
  Intronless MgD9Ds clone
atggcttcgtcatcttcctccgtgccggagttggctgccgccttccctgatggcactaccgacttcaagcccatgaggaacaccaagggctacgacgtcagcaagccgcacatttccgagacacctatgacactcaagaactggcataagcacgtcaactggctcaacaccaccttcatcttgtttgtgcccctggctggtctcatatccacttactgggtccctctgcagtggaagacggctgtatgggctgtcgtctactacttcaacaccggcctgggaattactgccggttaccaccgactttgggctcacagctcgtacaaggcctcgcttccgctcaaaatctaccttgccgccgttggcgctggtgccgtcgagggctccatcagatggtggtccaacggtcaccgcgcacaccaccgatacaccgataccgagaaggacccctactcagtccgcaagggtctcctgtactcacacatgggatggatgcttctgaagcagaaccccaagaagcagggccgcaccgacatcaccgacctgaacgaggaccccgttgtcgtttggcagcaccgcaacttcctcaagtgtgttatcttcatggccctcgtcttccccacacttgtggctggccttggctggggtgactactggggaggtttcatctacggaggtattctgcgtgtcttcttcgtccagcaggccaccttctgcgtcaactcgcttgcccactggctcggtgaccagcctttcgacgatcgcaactcgccgcgtgatcacgtcatcacagccctggtcacccttggagagggataccacaacttccaccacgagttcccttcggactaccgcaacgctattgagtggtaccagtatgaccccaccaagtggtcaatctggatctggaagcagcttggtcttgcccacaacctgaagcagttccgccaaaacgagattgagaagggacgcgtccagcagctgcagaagaagctcgaccagaagcgcgccaagcttgattggggtattcccttggagcagcttcccgttgttagctgggatgactttgttgagcagtccaagaacggaaaggcttggattgcagttgccggtgtcatccacgatgttggtgacttcatcaaggaccaccctggtggcagagctctcatcaactcggccattggcaaggacgcaaccgcaatcttcaacggcggtgtttacaaccactccaacgccgctcacaacctgctctcgactatgcgtgtgggtgttttgcgtggcggctgcgaggttgagatctggaagcgcgcccagtccgaaaacaaggacgtctcaaccgtcgttgattcttcgggtaaccgcatcgtccgcgcgggtgggcaagcgaccaaggtcgtccagcctgttccgggtgctcaggccgcgtga

SEQ ID NO: 5 - DNA, 1997 bp, Leptosphaeria nodorum
cccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggccgctctagaactagtggatcccccgggctgcaggaattcggcacgagtatgccttcccaccaggctgttgctggcatgcaggccatcgaccccgagtttgtcaagcagccgtctcctatggcgagcacctcggagcccaaccgcaactccaagtacgatcctaagaagccgcacattacagacatgcccatcacgcggtcaaactggtaccagcatgtcaactggctcaacgtcatcttcatcatcggcgtgcctctcgctggctgcgtcgccgccttctggacccctctgcagtggaagaccgctgcgtgggctgtcatctactatttctggactggcctcggtatcaccgccggataccatcgtctctgggcacacaagtcatacaacgccggtcttcctctgaggatctggctcgccgccgtcggcgctggtgctgttgagggttccatccgctggtggagccgtgaccaccgcgcccaccaccgctacaccgacaccaacaaggacccctacagtgtccgcaagggccttctctacagccatctcggatggatggtcatgaagcagaaccccaagcgtatcggccgcaccgacatcaccgacttgaacgaggaccccgttgtcgtctggcagcacaagaactacatcaaggccgtcgtcaccatgggcttgatctttccctctgccgtcgccggtctcatgtggggcgattggatgggtggcttcatctacgctggtatcctccgtatcttcttcgtccagcaggccaccttctgcgtcaactcgcttgctcactggctcggtgaccagcccttcgacgaccgcaactctcctcgtgaccacgtcattaccgctcttgtcactctcggagagggctaccacaacttccaccacgagttcccctccgactaccgcaacgccatcgagtggcaccagtacgaccctaccaagtggtccatctggctgtggagcaagctcggcctcgcctccaacctcaagcagttccgctccaacgaaatcgagaagggtcgtgtccagcagctccagaagaagattgaccagaagcgcgccaagctcgactggggtgtccctctcgaccagctgcctgtcatagaatgggacgactatgtcgagcaggccaagaacggccgtggtctcatcgctgtcgctggtgtcgttcatgacgttaccgacttcatcaacgagcaccccggtggcaagacgcttatcaagagcggcgttggcaaggatgccaccgccatgttcaacggcggtgtctacttccactccaacggagcccacaacctcctttctaccatgagggttggtgtcatccgcggtggctgtgaagttgagatctggaagcgcgctcagcgtgagaacaaggatgtcggtctggtcctggacgacgcaggcaacccaatcatcagggctggtaaccagattaccaaggttgcgcaacccattcagagtgctagtgcagcatagattggatcttcatcttcacgagcgatgtatggcgtttggttgtctctcttccttggcggacagagtaatattcaatttcttagcgatcgttagaaagcatcatggttacgatgctcagtcatgttagatggcgtatgtttgtagccttcctcgagtgattggstatgaaaagtagcctcacggcctagaccaagaatgaaaacattcacgatttcagaaaaaaaaaaaaaaaaaaactcgagggggggcc

SEQ ID NO: 6 - DNA, 39 bp, Artificial
  Forward primer Lnd9FAD2F
ggatccatggcggccttggacagcattccagaggataag

SEQ ID NO: 7 - DNA, 38 bp, Artificial
  Reverse primer Lnd9FAD2R
ccatggtcagttagctacgcagccacagccccctcaac

SEQ ID NO: 8 - DNA, 1370 bp, Leptosphaeria nodorum
ggatccatggcggccttggacagcattccagaggataaggctacctcgtcgaaatcgactcatattcaatatcaagaagtaacttttcggaactggtataagaagataaattggctcaacacgacgctggtggtgctcatacccgctcttggactctacctaacacgcaccacgccacttacacgacctacgctcatctggtccgtcctgtactacttctgcacagctttcggcatcacaggcggatatcatcgactatggagtcatcgcagctactccgctcgtctaccgctacgcttattcctagccttcacaggcgccggagccatccaaggtagtgctcgatggtggagcgcaaatcaccgcgcccaccaccgatggaccgacacaatgaaggacccctactccgttatgcgcggcctattattctcgcacatcggatggatggtattgaacagcgaccccaaagtcaaaggccgaacagacgtcagtgatctcgacagcgaccccgtcgtagtctggcagcacaagcactacggcaagtgcctgctgttcgccgcgtggatattccccatgatcgtagccggcctcggatggggagattggtggggaggccttgtctacgccggcatcattcgagcgtgtttcgtccagcaggcgacattttgcgtgaactctctcgcgcattggatcggcgagcagccgttcgacgacagacgcacgcctcgagaccacgttttgacagcgttggtaacgatgggagaaggatatcataacttccaccacgaattcccaagcgattatcgcaacgcgatcatctggtaccaatacgaccctaccaaatggctcatttacctcttctccctcggccccttccccctcgcatactcgctcaaaaccttccggtccaatgagattgaaaaagggcggttgcaacaacaacaaaaagccctggacaagaagcgctcaggacttgattggggcctacccctcttccaactccctgtcatatcgtgggacgacttccaagcgcgttgcaaagagtccggcgagatgctggttgctgtcgcaggtgtgattcacgacgtcagccagtttattgaagatcaccctggaggcaggagtttgattcggagtgcggtgggcaaagatgggacagggatgtttaatggaggcgtatatgagcacagtaatgcggcgcataatctgttgtcgacaatgagggtgggagtgcttagaggtgggcaggaggtggaggtgtggaagaagcagagagtggatgttttagggaagagcgacattttgagacaggttacgcgggtggagaggttggttgagggggctgtggctgcgtagctaactgaccatgg

SEQ ID NO: 9 - DNA, 1428 bp, Magnaporthe grisea; identical to SEQ ID NO: 4

SEQ ID NO: 10 - DNA, 1062 bp, Helicoverpa zea
atggctccaaatatatcggaggatgtgaacggggtgctcttcgagagtgatgcagcgacgccggacctggcgctgtccacgccgcctgtgcagaaggctgacaacaggcccaagcaactggtgtggaggaacatactactgttcgcgtatcttcacttagcggctctttacggaggttatctgttcctcttctcagctaaatggcagacagacatatttgcctacatcctgtatgtgatctccgggcttggtatcacggctggagcacatcgcctgtgggcccacaagtcctacaaagctaaatggcctctccgagttatcctggtcatctttaacacagtggcattccaggatgccgctatggactgggcgcgcgaccaccgcatgcatcacaagtactcggaaaccgatgctgatcctcataatgcgacccgaggattcttcttctctcacattggctggctgcttgtcaggaaacatcccgaccttaaggagaagggcaagggactcgacatgagcgacttacttgctgaccccattctcaggttccagaaaaaatactacctgatcctgatgcccttggcttgcttcgtgatgcctaccgtgattcctgtgtacttctggggtgaaacctggaccaacgcattctttgtggcggccatgttccgctacgcgttcatcctaaatgtgacgtggctcgtcaactctgccgctcacaagtggggagacaagccctacgacaaaagcattaagccttccgaaaacttgtcggtcgccatgttcgctctcggagaaggattccacaactaccaccacactttcccttgggactacaaaactgctgagctgggcaacaacaaactcaacttcactaccacctttattaacttcttcgctaaaattggctgggcttacgacctgaagacagtgtctgatgatatcgtcaagaacagggtgaagcgcactggtgacggctcccaccacctgtggggctggggagacgaaaatcaatccaaagaagaaattgatgccgctatcagaatcaatcctaaggacgattaa

SEQ ID NO: 11 - DNA, 1350 bp, Leptosphaeria nodorum
atggcggccttggacagcattccagaggataaggctacctcgtcgaaatcgactcatattcaatatcaagaagtaacttttcggaactggtataagaagataaattggctcaacacgacgctggtggtgctcatacccgctcttggactctacctaacacgcaccacgccacttacacgacctacgctcatctggtccgtcctgtactacttctgcacagctttcggcatcacaggcggatatcatcgactatggagtcatcgcagctactccgctcgtctaccgctacgcttattcctagccttcacaggcgccggagccatccaaggtagtgctcgatggtggagcgcaaatcaccgcgcccaccaccgatggaccgacacaatgaaggacccctactccgttatgcgcggcctattattctcgcacatcggatggatggtattgaacagcgaccccaaagtcaaaggccgaacagacgtcagtgatctcgacagcgaccccgtcgtagtctggcagcacaagcactacggcaagtgcctgctgttcgccgcgtggatattccccatgatcgtagccggcctcggatggggagattggtggggaggccttgtctacgccggcatcattcgagcgtgtttcgtccagcaggcgacattttgcgtgaactctctcgcgcattggatcggcgagcagccgttcgacgacagacgcacgcctcgagaccacgttttgacagcgttggtaacgatgggagaaggatatcataacttccaccacgaattcccaagcgattatcgcaacgcgatcatctggtaccaatacgaccctaccaaatggctcatttacctcttctccctcggccccttccccctcgcatactcgctcaaaaccttccggtccaatgagattgaaaaagggcggttgcaacaacaacaaaaagccctggacaagaagcgctcaggacttgattggggcctacccctcttccaactccctgtcatatcgtgggacgacttccaagcgcgttgcaaagagtccggcgagatgctggttgctgtcgcaggtgtgattcacgacgtcagccagtttattgaagatcaccctggaggcaggagtttgattcggagtgcggtgggcaaagatgggacagggatgtttaatggaggcgtatatgagcacagtaatgcggcgcataatctgttgtcgacaatgagggtgggagtgcttagaggtgggcaggaggtggaggtgtggaagaagcagagagtggatgttttagggaagagcgacattttgagacaggttacgcgggtggagaggttggttgagggggctgtggctgcgtag

SEQ ID NO: 12 - protein, 475 aa, Magnaporthe grisea
MASSSSSVPELAAAFPDGTTDFKPMRNTKGYDVSKPHISETPMTLKNWHKHVNWLNTTFILFVPLAGLISTYWVPLQWKTAVWAVVYYFNTGLGITAGYHRLWAHSSYKASLPLKIYLAAVGAGAVEGSIRWWSNGHRAHHRYTDTEKDPYSVRKGLLYSHMGWMLLKQNPKKQGRTDITDLNEDPVVVWQHRNFLKCVIFMALVFPTLVAGLGWGDYWGGFIYGGILRVFFVQQATFCVNSLAHWLGDQPFDDRNSPRDHVITALVTLGEGYHNFHHEFPSDYRNAIEWYQYDPTKWSIWIWKQLGLAHNLKQFRQNEIEKGRVQQLQKKLDQKRAKLDWGIPLEQLPVVSWDDFVEQSKNGKAWIAVAGVIHDVGDFIKDHPGGRALINSAIGKDATAIFNGGVYNHSNAAHNLLSTMRVGVLRGGCEVEIWKRAQSENKDVSTVVDSSGNRIVRAGGQATKVVQPVPGAQAA

SEQ ID NO: 13 - protein, 353 aa, Helicoverpa zea
MAPNISEDVNGVLFESDAATPDLALSTPPVQKADNRPKQLVWRNILLFAYLHLAALYGGYLFLFSAKWQTDIFAYILYVISGLGITAGAHRLWAHKSYKAKWPLRVILVIFNTVAFQDAAMDWARDHRMHHKYSETDADPHNATRGFFFSHIGWLLVRKHPDLKEKGKGLDMSDLLADPILRFQKKYYLILMPLACFVMPTVIPVYFWGETWTNAFFVAAMFRYAFILNVTWLVNSAAHKWGDKPYDKSIKPSENLSVAMFALGEGFHNYHHTFPWDYKTAELGNNKLNFTTTFINFFAKIGWAYDLKTVSDDIVKNRVKRTGDGSHHLWGWGDENQSKEEIDAAIRINPKDD

SEQ ID NO: 14 - protein, 449 aa, Leptosphaeria nodorum
MAALDSIPEDKATSSKSTHIQYQEVTFRNWYKKINWLNTTLVVLIPALGLYLTRTTPLTRPTLIWSVLYYFCTAFGITGGYHRLWSHRSYSARLPLRLFLAFTGAGAIQGSARWWSANHRAHHRWTDTMKDPYSVMRGLLFSHIGWMVLNSDPKVKGRTDVSDLDSDPVVVWQHKHYGKCLLFAAWIFPMIVAGLGWGDWWGGLVYAGIIRACFVQQATFCVNSLAHWIGEQPFDDRRTPRDHVLTALVTMGEGYHNFHHEFPSDYRNAIIWYQYDPTKWLIYLFSLGPFPLAYSLKTFRSNEIEKGRLQQQQKALDKKRSGLDWGLPLFQLPVISWDDFQARCKESGEMLVAVAGVIHDVSQFIEDHPGGRSLIRSAVGKDGTGMFNGGVYEHSNAAHNLLSTMRVGVLRGGQEVEVWKKQRVDVLGKSDILRQVTRVERLVEGAVAA

SEQ ID NO: 15 - DNA, 1428 bp, Artificial
  canola-optimized desaturase sequence
atggccagcagttcttcaagtgtgccagaacttgccgcagctttccctgatgggacaacggacttcaaacccatgaggaacaccaaaggctatgatgtctccaaacctcacatctctgaaacaccgatgactttgaagaactggcacaaacatgtgaactggctcaacaccacattcattctctttgttccactggctgggttgatctcaacctattgggttcctcttcaatggaaaactgcagtgtgggcagttgtgtactacttcaacactggacttgggatcactgctggctaccatagattgtgggcacattcctcttacaaggccagcttgcctctcaaaatctaccttgccgcagttggtgctggagccgttgaaggttccataagatggtggagcaacggacacagagcacatcacagatacacagacacagagaaagatccttactcagtgaggaagggattgctctacagccacatgggttggatgctcttgaagcagaatccaaagaagcaagggaggacggacattactgatctgaatgaggacccagttgtggtctggcaacataggaactttctcaagtgtgtgatcttcatggctttggtctttcccacccttgttgctggcctgggatggggagactactggggaggtttcatctatggagggatcttgagagtgttctttgttcagcaagccaccttctgtgtcaactcacttgcacattggcttggtgatcaaccgtttgatgacagaaactctccacgtgaccatgtcataactgctcttgtcacgctgggtgaaggctatcacaactttcaccatgagtttccgtcagactatagaaatgcgattgagtggtatcagtatgaccccacgaagtggagcatttggatttggaagcaacttggacttgctcacaatctcaagcagttcagacagaatgagatagagaagggaagggttcaacagttgcagaagaaactggatcagaagagagcgaaacttgattggggaataccgttggaacaactccctgttgtgtcttgggatgactttgttgaacagtcaaagaatggcaaggcatggattgctgttgctggtgtcattcacgatgttggtgacttcatcaaggatcatcctggtggacgtgctctcatcaactctgcgattggcaaagatgccacagcgatcttcaatggaggtgtctacaatcattcaaatgccgcacacaaccttctctccaccatgagggttggtgtcctccgtggagggtgcgaagtggagatatggaaacgtgctcaaagtgagaacaaagatgtctctactgtggttgatagttctggcaaccgtattgtgagagctggtggacaagctaccaaagtggttcagccagtccctggtgctcaagcagcttga

SEQ ID NO: 16 - DNA, 1062 bp, Artificial
  Canola-optimized desaturase sequence
atggctcccaacatttctgaggatgtcaatggtgttctttttgagtcagatgcggcaacccctgatttggctctttccacaccacctgtgcaaaaagctgacaacagacccaagcaacttgtgtggaggaacattttgcttttcgcttacttgcacctcgcagctctctacggaggctatttgtttctcttcagtgcaaaatggcagaccgacattttcgcttacattctttatgtcatctctggactggggataactgctggggcacatagactctgggctcacaagtcatacaaagccaagtggccactcagagttatactggtcatcttcaacacggttgcctttcaagacgctgctatggattgggctcgtgaccatagaatgcatcacaagtacagcgagaccgacgcggacccacacaatgcaacgagaggtttcttcttctctcacattggctggcttcttgttaggaaacatcctgatctgaaagaaaaagggaagggactcgacatgagtgatctccttgctgatccaatactccgttttcagaagaagtactatctgatcctcatgcctctggcctgttttgtgatgccaaccgttatcccggtttacttttggggagaaacttggacaaatgctttcttcgtggcagccatgttccgttatgctttcatcctgaatgttacctggttggtgaactctgccgcacacaagtggggagacaaaccctatgacaagtccatcaagccttccgaaaacctttcagttgcgatgtttgctttgggagaaggatttcacaattaccatcacacttttccgtgggactacaagacagcagagcttggaaacaacaagttgaacttcacaacaacgttcatcaatttctttgcgaaaatcggttgggcctatgatttgaagactgtgagtgatgacattgtcaagaacagggtcaagagaactggcgatggaagccatcatctctggggctggggtgatgagaatcagagcaaagaagagatagatgcagccattaggatcaaccctaaagacgattga

SEQ ID NO: 17 - DNA, 1350 bp, Artificial
  Canola-optimized desaturase sequence
atggctgcacttgatagcatccctgaggacaaagcaactagctccaagtcaacccacatacagtaccaagaggtcacgtttaggaactggtacaagaaaatcaactggctcaacacgacccttgttgtcctcattcctgctcttgggttgtacttgacgagaaccacacctctcaccagacctaccctcatttggtctgttctctactatttctgtacagcgtttggcatcactggtggctaccacagactttggtcccataggtcttacagtgcgaggttgccattgagactcttcctggctttcactggagctggtgcgatccaaggttctgcaagatggtggtcagccaatcatagggcacatcaccgttggacggacaccatgaaggacccctactctgtgatgagaggactgctgttctcccacataggttggatggttctcaactctgatccaaaggtcaaaggcagaacagatgtttctgatcttgactctgatcccgtcgttgtgtggcaacacaaacactatggcaagtgtttgctctttgccgcttggatctttccgatgatagtggctgggctgggttggggagattggtggggtggacttgtctatgctggcatcatacgtgcctgctttgttcagcaagccactttctgtgtcaactcattggcacattggataggtgaacaaccgtttgatgacagacgtactccaagggatcatgttctgactgcgttggtcacaatgggagaaggataccacaacttccaccatgagtttccgagtgactacagaaatgccatcatttggtatcagtatgaccctacaaagtggctcatctatctcttcagcttgggtcccttcccattggcctactctctcaagaccttccgttccaatgagattgagaaaggaaggcttcagcaacagcaaaaggctcttgacaagaaaagaagtggtcttgattggggacttcctctcttccagcttccagtgatctcatgggatgactttcaagctcgttgcaaagaaagtggagagatgcttgttgctgttgctggagtgatccatgatgtctcccagttcattgaagatcatcctggtgggaggagcctcattagaagtgctgttgggaaagatgggactggcatgttcaatggtggagtgtatgaacattcaaacgccgcacacaacttgctgagcacaatgagagttggagtcttgagaggtggacaagaagtggaggtttggaagaaacagagggtggatgttcttgggaagtcagacattcttcgtcaagtgacaagggtggagcgtctggtggaaggagctgttgcagcgtga

SEQ ID NO: 18 - DNA, 24 bp, Artificial
  Primer MAS414
tgaagcattccataagccgtcacg

SEQ ID NO: 19 - DNA, 24 bp, Artificial
  Primer MAS415
gaaattatcacgcttccgcacacg

SEQ ID NO: 20 - DNA, 24 bp, Artificial
  Primer MAS413
tgggctgaattgaagacatgctcc

SEQ ID NO: 21 - DNA, 25 bp, Artificial
  Primer arw008
acacctctcaccagacctaccctca

SEQ ID NO: 22 - DNA, 22 bp, Artificial
  Primer arw009
cacacaacgacgggatcagagt

SEQ ID NO: 23 - DNA, 24 bp, Artificial
  Primer arw010
caagtcatacaaagccaagtggcc

SEQ ID NO: 24 - DNA, 22 bp, Artificial
  Primer arw011
taacggaacatggctgccacga

SEQ ID NO: 25 - DNA, 33 bp, Artificial
  Primer AntiLnD9DS2F
catatgttcgacgacagacgcacgcctcgagac

SEQ ID NO: 26 - DNA, 35 bp, Artificial
  Primer AntiLnD9DS2Rh
ggatccgcagccacagccccctcaaccaacctctc

SEQ ID NO: 27 - DNA, 36 bp, Artificial
  Primer AntiMgD9DSF
catatgttcgacgatcgcaactcgccgcgtgatcac

SEQ ID NO: 28 - DNA, 32 bp, Artificial
  Primer AntiMgD9DSRh
ggatccgcggcctgagcacccggaacaggctg

SEQ ID NO: 29 - DNA, 30 bp, Artificial
  Primer AntiHzD9DSF
catatgtatgacaagtccatcaagccttcc

SEQ ID NO: 30 - DNA, 35 bp, Artificial
  Primer AntiHzD9DSRh
ggatcctcgtctttagggttgatcctaatggctgc

SEQ ID NO: 31 - DNA, 24 bp, Artificial
  Forward primer for target AnD9Ds
ggacttctctactctcaccttgga

SEQ ID NO: 32 - DNA, 20 bp, Artificial
  Reverse primer for target AnD9Ds
tccgatcctctttgggttct

SEQ ID NO: 33 - DNA, 18 bp, Artificial
  Forward primer for target HzD9Ds
gacccacacaatgcaacg

SEQ ID NO: 34 - DNA, 21 bp, Artificial
  Reverse primer for target HzD9Ds
cctaacaagaagccagccaat

SEQ ID NO: 35 - DNA, 20 bp, Artificial
  Forward primer for target LnD9Ds
gttctgactgcgttggtcac

SEQ ID NO: 36 - DNA, 20 bp, Artificial
  Reverse primer for target LnD9Ds
cggaaactcatggtggaagt

SEQ ID NO: 37 - DNA, 21 bp, Artificial
  Forward primer for target Actin
ctactggtattgtgctcgact

SEQ ID NO: 38 - DNA, 22 bp, Artificial
  Reverse primer for target Actin
ctctctcggtgagaatcttcat

SEQ ID NO: 39 - DNA, 22 bp, Artificial
  Actin probe
cacgctatcctccgtctcgatc

SEQ ID NO: 40 - DNA, 13 bp, Artificial
  Kozak sequence
ggatccaacaatg

SEQ ID NO: 41 - DNA, 13 bp, Artificial
  Kozak sequence
acaaccaaaaatg

SEQ ID NO: 42 - DNA, 18 bp, Artificial
  Kozak sequence
acaaccaacctaccatgg

SEQ ID NO: 43 - DNA, 14 bp, Artificial
  Kozak sequence
acaaccaaaaaatg

SEQ ID NO: 44 - DNA, 1350 bp, Artificial
  Canola-optimized desaturase
atggctgctcttgattctatcccagaggataaggctacctcttctaagtctacccacatccaataccaagaagttaccttcaggaactggtacaagaagatcaactggcttaacaccacccttgttgttcttatcccagctcttggactttaccttaccaggaccaccccacttaccaggccaacccttatctggtctgttctttactacttctgcaccgctttcggaataaccggaggataccacaggctttggtctcacaggtcttactctgctaggcttccacttaggcttttccttgctttcaccggagctggagctatccaaggatctgctagatggtggtctgctaaccacagggctcaccacaggtggaccgataccatgaaggacccatactctgttatgaggggacttcttttctctcacatcggatggatggttcttaactctgatccaaaggttaagggaaggaccgatgtttctgatcttgattctgatccagttgttgtttggcaacacaagcactacggaaagtgccttcttttcgctgcttggatcttcccaatgatcgttgctggacttggatggggagattggtggggaggacttgtttacgctggaatcatcagggcttgcttcgttcaacaagctaccttctgcgttaactctcttgctcactggatcggagagcaaccattcgacgataggaggaccccaagggatcacgttcttaccgctcttgttaccatgggagagggataccacaacttccaccacgagttcccatctgattacaggaacgctatcatctggtaccaatacgatccaaccaagtggcttatctaccttttctctcttggaccattcccacttgcttactctcttaagaccttcaggtctaacgagatcgagaagggaaggcttcaacaacaacaaaaggctcttgataagaagaggtctggacttgattggggacttccacttttccaacttccagttatctcttgggatgatttccaagctaggtgcaaggagtctggagagatgcttgttgctgttgctggagttatccacgatgtttctcaattcatcgaggatcacccaggaggaaggtctcttatcaggtctgctgttggaaaggatggaaccggaatgttcaacggaggagtttacgagcactctaacgctgctcacaaccttctttctaccatgagggttggagttcttaggggaggacaagaggttgaggtttggaagaagcaaagggttgatgttcttggaaagtcagatatccttaggcaagttaccagggttgagaggcttgttgagggagctgttgctgcttga

SEQ ID NO: 45 - DNA, 1062 bp, Artificial
  Canola-optimized desaturase
atggctccaaacatctctgaggatgttaacggagttcttttcgagtctgatgctgctaccccagatcttgctctttctaccccaccagttcaaaaggctgataacaggccaaagcaacttgtttggaggaacatccttcttttcgcttaccttcaccttgctgctctttacggaggataccttttccttttctctgctaagtggcaaaccgatatcttcgcttacatcctttacgttatctctggacttggaataaccgctggagcacacaggctttgggctcacaagtcttacaaggctaagtggccacttagggttatccttgttatcttcaacaccgttgctttccaagacgctgctatggattgggctagggatcacaggatgcaccacaagtactctgagaccgacgctgatccacacaacgctaccaggggattcttcttctctcacatcggatggcttcttgttaggaagcacccagatcttaaggagaagggaaagggacttgatatgtctgatcttcttgctgatccaatccttaggttccaaaagaagtactaccttatccttatgccacttgcttgcttcgttatgccaaccgttatcccagtttacttctggggagagacctggaccaacgctttcttcgttgctgctatgttcaggtacgctttcatccttaacgttacctggcttgttaactctgctgctcacaagtggggagataagccatacgataagtctatcaagccatctgagaacctttctgttgctatgttcgctcttggagagggattccacaactaccaccacaccttcccatgggattacaagaccgctgagcttggaaacaacaagcttaacttcaccaccaccttcatcaacttcttcgctaagatcggatgggcttacgatcttaagaccgtttctgatgatatcgttaagaacagggttaagaggaccggagatggatcacaccacctttggggatggggagatgagaaccaatctaaggaggagatcgatgctgctatcaggatcaacccaaaggatgattga

SEQ ID NO: 46 - protein, 10 aa, Artificial
  Myc tag
EQKLISEEDL

SEQ ID NO: 47 - protein, 9 aa, Artificial
  Hemagglutin tag
YPYDVPDYA

SEQ ID NO: 48 - DNA, 1368 bp, Aspergillus nidulans
atgtctgctccaaccgctgacatcagggctagggctccagaggctaagaaggttcacatcgctgataccgctatcaacaggcacaattggtacaagcacgtgaactggctcaacgtcttcctcatcatcggaatcccactctacggatgcatccaagctttctgggttccacttcaactcaagaccgctatctgggctgtgatctactacttcttcaccggacttggaatcaccgctggataccacaggctttgggctcactgctcttactctgctactcttccacttaggatctggcttgctgctgttggaggaggagctgttgagggatctatcagatggtgggctagggatcacagggctcatcataggtacaccgataccgacaaggacccatactctgttaggaagggacttctctactctcaccttggatggatggtgatgaagcagaacccaaagaggatcggaaggaccgacatctctgatctcaacgaggacccagttgttgtttggcaacacaggaactacctcaaggttgtgttcaccatgggacttgctgttccaatgcttgttgctggacttggatggggagattggcttggaggattcgtgtacgctggaatccttaggatcttcttcgttcaacaagctaccttctgcgtgaactctcttgctcactggcttggagatcaaccattcgatgataggaactctcctagggatcacgtgatcaccgctcttgttacccttggagagggataccacaacttccaccacgagttcccatctgactacaggaacgctatcgagtggcaccagtacgatcctaccaagtggtctatctgggcttggaagcaacttggattggcttacgatctcaagaagttcagggctaacgagatcgagaagggaagggttcaacaacttcagaagaagcttgataggaagagggctactcttgattggggaaccccacttgatcaacttccagtgatggaatgggatgactacgttgagcaagctaagaacggaaggggacttgttgctatcgctggagttgttcacgatgttaccgacttcatcaaggatcacccaggaggaaaggctatgatctcttctggaatcggaaaggatgctaccgctatgttcaacggaggagtgtactaccactctaacgcagctcacaaccttcttagcaccatgagggtgggagtgatcaggggaggatgcgaggttgagatctggaagagggctcagaaggagaacgttgagtacgttagggatggatctggacaaagggtgatcagggctggagagcaaccaaccaagatcccagagccaatcccaaccgctgatgctgcttga

SEQ ID NO: 49 - DNA, 1368 bp, Artificial
  AnD9DS v3 silent mutant
atgtctgctccaaccgctgacatcagggctagggctccagaggctaagaaggttcacatcgctgataccgctatcaacaggcacaattggtacaagcacgtgaactggctcaacgtcttcctcatcatcggaatcccactctacggatgcatccaagctttctgggttccacttcaactcaagaccgctatctgggctgtgatctactacttcttcaccggacttggaatcaccgctggataccacaggctttgggctcactgctcatactctgctactcttccacttaggatctggcttgctgctgttggaggaggagctgttgagggatctatcagatggtgggctagggatcacagggctcatcataggtacaccgataccgacaaggacccatactctgttaggaagggacttctctactctcaccttggatggatggtgatgaagcagaacccaaagaggatcggaaggaccgacatctctgatctcaacgaggacccagttgttgtttggcaacacaggaactacctcaaggttgtgttcaccatgggacttgctgttccaatgcttgttgctggacttggatggggagattggcttggaggattcgtgtacgctggaatccttaggatcttcttcgttcaacaagctaccttctgcgtgaactctcttgctcactggcttggagatcaaccattcgatgataggaactctcctagggatcacgtgatcaccgctcttgttacccttggagagggataccacaacttccaccacgagttcccatctgactacaggaacgctatcgagtggcaccagtacgatcctaccaagtggtctatctgggcttggaagcaacttggattggcttacgatctcaagaagttcagggctaacgagatcgagaagggaagggttcaacaacttcagaagaagcttgataggaagagggctactcttgattggggaaccccacttgatcaacttccagtgatggaatgggatgactacgttgagcaagctaagaacggaaggggacttgttgctatcgctggagttgttcacgatgttaccgacttcatcaaggatcacccaggaggaaaggctatgatctcttctggaatcggaaaggatgctaccgctatgttcaacggaggagtgtactaccactctaacgcagctcacaaccttcttagcaccatgagggtgggagtgatcaggggaggatgcgaggttgagatctggaagagggctcagaaggagaacgttgagtacgttagggatggatctggacaaagggtgatcagggctggagagcaaccaaccaagatcccagagccaatcccaaccgctgatgctgcttga

SEQ ID NO: 50 - protein, 455 aa, Aspergillus nidulans
MSAPTADIRARAPEAKKVHIADTAINRHNWYKHVNWLNVFLIIGIPLYGCIQAFWVPLQLKTAIWAVIYYFFTGLGITAGYHRLWAHCSYSATLPLRIWLAAVGGGAVEGSIRWWARDHRAHHRYTDTDKDPYSVRKGLLYSHLGWMVMKQNPKRIGRTDISDLNEDPVVVWQHRNYLKVVFTMGLAVPMLVAGLGWGDWLGGFVYAGILRIFFVQQATFCVNSLAHWLGDQPFDDRNSPRDHVITALVTLGEGYHNFHHEFPSDYRNAIEWHQYDPTKWSIWAWKQLGLAYDLKKFRANEIEKGRVQQLQKKLDRKRATLDWGTPLDQLPVMEWDDYVEQAKNGRGLVAIAGVVHDVTDFIKDHPGGKAMISSGIGKDATAMFNGGVYYHSNAAHNLLSTMRVGVIRGGCEVEIWKRAQKENVEYVRDGSGQRVIRAGEQPTKIPEPIPTADAA

SEQ ID NO: 51 - protein, 455 aa, Aspergillus nidulans
MSAPTADIRARAPEAKKVHIADTAINRHNWYKHVNWLNVFLIIGIPLYGCIQAFWVPLQLKTAIWAVIYYFFTGLGITAGYHRLWAHCSYSATLPLRIWLAAVGGGAVEGSIRWWARDHRAHHRYTDTDKDPYSVRKGLLYSHLGWMVMKQNPKRIGRTDISDLNEDPVVVWQHRNYLKVVFTMGLAVPMLVAGLGWGDWLGGFVYAGILRIFFVQQATFCVNSLALWLGDQPFDDRNSPRDHVITALVTLGEGYHNFHHEFPSDYRNAIEWHQYDPTKWSIWAWKQLGLAYDLKKFRANEIEKGRVQQLQKKLDRKRATLDWGTPLDQLPVMEWDDYVEQAKNGRGLVAIAGVVHDVTDFIKDHPGGKAMISSGIGKDATAMFNGGVYYHSNAAHNLLSTMRVGVIRGGCEVEIWKRAQKENVEYVRDGSGQRVIRAGEQPTKIPEPIPTADAA

SEQ ID NO: 52 - protein, 510 aa, Saccharomyces cerevisiae
MPTSGTTIELIDDQFPKDDSASSGIVDEVDLTEANILATGLNKKAPRIVNGFGSLMGSKEMVSVEFDKKGNEKKSNLDRLLEKDNQEKEEAKTKIHISEQPWTLNNWHQHLNWLNMVLVCGMPMIGWYFALSGKVPLHLNVFLFSVFYYAVGGVSITAGYHRLWSHRSYSAHWPLRLFYAIFGCASVEGSAKWWGHSHRIHHRYTDTLRDPYDARRGLWYSHMGWMLLKPNPKYKARADITDMTDDWTIRFQHRHYILLMLLTAFVIPTLICGYFFNDYMGGLIYAGFIRVFVIQQATFCINSMAHYIGTQPFDDRRTPRDNWITAIVTFGEGYHNFHHEFPTDYRNAIKWYQYDPTKVIIYLTSLVGLAYDLKKFSQNAIEEALIQQEQKKINKKKAKINWGPVLTDLPMWDKQTFLAKSKENKGLVIISGIVHDVSGYISEHPGGETLIKTALGKDATKAFSGGVYRHSNAAQNVLADMRVAVIKESKNSAIRMASKRGEIYETGKFF

SEQ ID NO: 53 - DNA, 13227 bp, Artificial
  Plasmid pDAB7309
ggactagtccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtaggtaccagatctggatcccaaaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatctacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccaaatctgagtagttagcttaatcacctagagctcgatcggcggcaatagcttcttagcgccatcccgggttgatcctatctgtgttgaaatagttgcggtgggcaaggctctctttcagaaagacaggcggccaaaggaacccaaggtgaggtgggctatggctctcagttccttgtggaagcgcttggtctaaggtgcagaggtgttagcgggatgaagcaaaagtgtccgattgtaacaagatatgttgatcctacgtaaggatattaaagtatgtattcatcactaatataatcagtgtattccaatatgtactacgatttccaatgtctttattgtcgccgtatgtaatcggcgtcacaaaataatccccggtgactttcttttaatccaggatgaaataatatgttattataatttttgcgatttggtccgttataggaattgaagtgtgcttgaggtcggtcgccaccactcccatttcataattttacatgtatttgaaaaataaaaatttatggtattcaatttaaacacgtatacttgtaaagaatgatatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatggtgtaatacataggaattcgtttaaacgatctgcgtctaattttcggtccaacttgcacaggaaagacgtcgaccgcggtagctcttgcccagcagactgggcttccagtcctttcgctcgatcgggtccaatgttgtcctcagctgtgaaccggaagcggacgaccaacagtggaagaactgaaaggaacgagccgtctataccttgatgatcggcctctggtgaagggtatcatcgcagccaagcaagctcatgaaaggctgatgggggaggtgtataattatgaggcccacggcgggcttattctttagggaggatctatctcgttgctcaagtgcatggcgcaaagcagttattggagtgcggattttcgttggcatattattcgccacgagttagcagacgaagagaccttcatgaacgtggccaaggccagagttaagcagatgttacgccctgctgcaggcctttctattatccaatagttggttgatctttggaaagagcctcggctgaggcccatactgaaagagatcgatggatatcgatatgccatgttgtttgctagccagaaccagatcacatccgatatgctattgcagcttgacgcagatatggaggataagttgattcatgggatcgctcaggagtagctcatccatgcacgccgacaagaacagaaattccgtcgagttaacgcagccgcttacgacggattcgaaggtcatccattcggaatgtattagtttgcaccagctccgcgtcacacctgtcttcatttgaataagatgttagcaattgtttttagctttgtcttgttgtggcagggcggcaagtgcttcagacatcattctgttttcaaattttatgctggagaacagcttcttaattcctttggaaataatagactgcgtcttaaaattcagatgtctggatatagatatgattgtaaaataacctatttaagtgtcatttagaacataagttttatgaatgttcttccattttcgtcatcgaacgaataagagtaaatacaccttttttaacattacaaataagttcttatacgttgtttatacaccgggaatcatttccattattttcgcgcaaaagtcacggatattcgtgaaagcgacataaactgcgaaatttgcggggagtgtcttgagtttgcctcgaggctagcgcatgcacatagacacacacatcatctcattgatgcttggtaataattgtcattagattgtttttatgcatagatgcactcgaaatcagccaattttagacaagtatcaaacggatgtgacttcagtacattaaaaacgtccgcaatgtgttattaagttgtctaagcgtcaatttgatttacaattgaatatatcctgccccagccagccaacagctcgatttacaattgaatatatcctgccggccggcccacgcgtgtcgaggaattctgatctggcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttaaacatcatccgtcggatggcgttgcgagagaagcagtcgatccgtgagatcagccgacgcaccgggcaggcgcgcaacacgatcgcaaagtatttgaacgcaggtacaatcgagccgacgttcacgcggaacgaccaagcaagcttggctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaagggtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagcccccgagcgcttagtgggaatttgtaccccttatcgaaccgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtcttgcgatggcatttcgatccattcccattccgcgctcaagatggcttcccctcggcagttcatcagggctaaatcaatctagccgacttgtccggtgaaatgggctgcactccaacagaaacaatcaaacaaacatacacagcgacttattcacacgagctcaaattacaacggtatatatcctgccagtcagcatcatcacaccaaaagttaggcccgaatagtttgaaattagaaagctcgcaattgaggtctacaggccaaattcgctcttagccgtacaatattactcaccggatcctaaccggtgtgatcatgggccgcgattaaaaatctcaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaacctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaatgaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggccaccgcttaattaaggcgcgccatgcccgggcaagcggccgcacaagtttgtacaaaaaagctgaacgagaaacgtaaaatgatataaatatcaatatattaaattagattttgcataaaaaacagactacataatactgtaaaacacaacatatccagtcactatgaatcaactacttagatggtattagtgacctgtagtcgaccgacagccttccaaatgttcttcgggtgatgctgccaacttagtcgaccgacagccttccaaatgttcttctcaaacggaatcgtcgtatccagcctactcgctattgtcctcaatgccgtattaaatcataaaaagaaataagaaaaagaggtgcgagcctcttttttgtgtgacaaaataaaaacatctacctattcatatacgctagtgtcatagtcctgaaaatcatctgcatcaagaacaatttcacaactcttatacttttctcttacaagtcgttcggcttcatctggattttcagcctctatacttactaaacgtgataaagtttctgtaatttctactgtatcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagcccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcatacctctttttgacatacttcgggtatacatatcagtatatattcttataccgcaaaaatcagcgcgcaaatacgcatactgttatctggcttttagtaagccggatccacgcggcgtttacgccccccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttagtcgactacaggtcactaataccatctaagtagttgattcatagtgactggatatgttgtgttttacagtattatgtagtctgttttttatgcaaaatctaatttaatatattgatatttatatcattttacgtttctcgttcagctttcttgtacaaagtggttgcggccgcttaattaaatttaaattcaattaatgcaatcttgattttcaacaacgaaggtaatggcgtaaaagaaaaaatgtatgttattgtattgatctttcatgatgttgaagcgtgccataatatgatgatgtataattaaaatattaactgtcgcattttattgaaatggcactgttatttcaaccatatctttgattctgttacatgacacgactgcaagaagtaaataatagacgccgttgttaaagaattgctatcatatgtgcctaactagagggaatttgagcgtcagacctaatcaaatattacaaaatatctcactctgtcgccagcaatggtgtaatcagcgcagacaaatggcgtaaagatcgcggaaaaacctccccgagtggcatgatagctgcctctgtattgctgatttagtcagccttatttgacttaagggtgccctcgttagtgacaaattgctttcaaggagacagccatgccccacactttgttgaaaaacaaattgcctttggggagacggtaaagccagttgctcttcaataaggaatgtcgaggaggcaatgtaaccgcctctggtagtacacttctctaatccaaaaatcaatttgtattcaagataccgcaaaaaacttatggtttaaaccctgca

SEQ ID NO: 54 - DNA, 10247 bp, Artificial
  Plasmid pDAB7318
cgcgccgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgcggccgcactaggtttaaactctagaagctaggaattcaaacaaagaagcgatcgcgcggccgccattgtactcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataatgatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatgatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataagatttatggtggactaattttcatatatttcttattgcttttaccttttcttggtatgtaagtccgtaactggaattactgtgggttgccatgacactctgtggtcttttggttcatgcatggatgcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctaggatccaacaatgtctgctccaaccgctgacatcagggctagggctccagaggctaagaaggttcacatcgctgataccgctatcaacaggcacaattggtacaagcacgtgaactggctcaacgtcttcctcatcatcggaatcccactctacggatgcatccaagctttctgggttccacttcaactcaagaccgctatctgggctgtgatctactacttcttcaccggacttggaatcaccgctggataccacaggctttgggctcactgctcatactctgctactcttccacttaggatctggcttgctgctgttggaggaggagctgttgagggatctatcagatggtgggctagggatcacagggctcatcataggtacaccgataccgacaaggacccatactctgttaggaagggacttctctactctcaccttggatggatggtgatgaagcagaacccaaagaggatcggaaggaccgacatctctgatctcaacgaggacccagttgttgtttggcaacacaggaactacctcaaggttgtgttcaccatgggacttgctgttccaatgcttgttgctggacttggatggggagattggcttggaggattcgtgtacgctggaatccttaggatcttcttcgttcaacaagctaccttctgcgtgaactctcttgctcactggcttggagatcaaccattcgatgataggaactctcctagggatcacgtgatcaccgctcttgttacccttggagagggataccacaacttccaccacgagttcccatctgactacaggaacgctatcgagtggcaccagtacgatcctaccaagtggtctatctgggcttggaagcaacttggattggcttacgatctcaagaagttcagggctaacgagatcgagaagggaagggttcaacaacttcagaagaagcttgataggaagagggctactcttgattggggaaccccacttgatcaacttccagtgatggaatgggatgactacgttgagcaagctaagaacggaaggggacttgttgctatcgctggagttgttcacgatgttaccgacttcatcaaggatcacccaggaggaaaggctatgatctcttctggaatcggaaaggatgctaccgctatgttcaacggaggagtgtactaccactctaacgcagctcacaaccttcttagcaccatgagggtgggagtgatcaggggaggatgcgaggttgagatctggaagagggctcagaaggagaacgttgagtacgttagggatggatctggacaaagggtgatcagggctggagagcaaccaaccaagatcccagagccaatcccaaccgctgatgctgcttgagtagttagcttaatcacctaggtcaccagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaactttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaattttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacccttatacaaaaccccaaaaacaagtttcctagcaccctaccaacgaattcgcggccgctttcctgcatgacatcgtcctgcagagccaagcgcatgcttaattaaactagtctcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataatgatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatgatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataagatttatggtggactaattttcatatatttcttattgcttttaccttttcttggtatgtaagtccgtaactggaattactgtgggttgccatgacactctgtggtcttttggttcatgcatggatcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctaggtaccctgcagggatccaacaatggctgcacttgatagcatccctgaggacaaagcaactagctccaagtcaacccacatacagtaccaagaggtcacgtttaggaactggtacaagaaaatcaactggctcaacacgacccttgttgtcctcattcctgctcttgggttgtacttgacgagaaccacacctctcaccagacctaccctcatttggtctgttctctactatttctgtacagcgtttggcatcactggtggctaccacagactttggtcccataggtcttacagtgcgaggttgccattgagactcttcctggctttcactggagctggtgcgatccaaggttctgcaagatggtggtcagccaatcatagggcacatcaccgttggacggacaccatgaaggacccctactctgtgatgagaggactgctgttctcccacataggttggatggttctcaactctgatccaaaggtcaaaggcagaacagatgtttctgatcttgactctgatcccgtcgttgtgtggcaacacaaacactatggcaagtgtttgctctttgccgcttggatctttccgatgatagtggctgggctgggttggggagattggtggggtggacttgtctatgctggcatcatacgtgcctgctttgttcagcaagccactttctgtgtcaactcattggcacattggataggtgaacaaccgtttgatgacagacgtactccaagggatcatgttctgactgcgttggtcacaatgggagaaggataccacaacttccaccatgagtttccgagtgactacagaaatgccatcatttggtatcagtatgaccctacaaagtggctcatctatctcttcagcttgggtcccttcccattggcctactctctcaagaccttccgttccaatgagattgagaaaggaaggcttcagcaacagcaaaaggctcttgacaagaaaagaagtggtcttgattggggacttcctctcttccagcttccagtgatctcatgggatgactttcaagctcgttgcaaagaaagtggagagatgcttgttgctgttgctggagtgatccatgatgtctcccagttcattgaagatcatcctggtgggaggagcctcattagaagtgctgttgggaaagatgggactggcatgttcaatggtggagtgtatgaacattcaaacgccgcacacaacttgctgagcacaatgagagttggagtcttgagaggtggacaagaagtggaggtttggaagaaacagagggtggatgttcttgggaagtcagacattcttcgtcaagtgacaagggtggagcgtctggtggaaggagctgttgcagcgtgatgagtagttagcttaatcacctagagctcggtcacctcgagtatcaaaatctatttagaaatacacaatattttgttgcaggcttgctggagaatcgatctgctatcataaaaattacaaaaaaattttatttgcctcaattattttaggattggtattaaggacgcttaaattatttgtcgggtcactacgcatcattgtgattgagaagatcagcgatacgaaatattcgtagtactatcgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaaggttgg

SEQ ID NO: 55 - DNA, 6058 bp, Artificial
  Plasmid pDAB7320
aattcgcggccgctttcctgcatgacatcgtcctgcagagccaagcgcatgcttaattaaactagtctcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataatgatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatgatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataagatttatggtggactaattttcatatatttcttattgcttttaccttttcttggtatgtaagtccgtaactggaattactgtgggttgccatgacactctgtggtcttttggttcatgcatggatcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctaggtaccctgcagggatccaacaatggctgcacttgatagcatccctgaggacaaagcaactagctccaagtcaacccacatacagtaccaagaggtcacgtttaggaactggtacaagaaaatcaactggctcaacacgacccttgttgtcctcattcctgctcttgggttgtacttgacgagaaccacacctctcaccagacctaccctcatttggtctgttctctactatttctgtacagcgtttggcatcactggtggctaccacagactttggtcccataggtcttacagtgcgaggttgccattgagactcttcctggctttcactggagctggtgcgatccaaggttctgcaagatggtggtcagccaatcatagggcacatcaccgttggacggacaccatgaaggacccctactctgtgatgagaggactgctgttctcccacataggttggatggttctcaactctgatccaaaggtcaaaggcagaacagatgtttctgatcttgactctgatcccgtcgttgtgtggcaacacaaacactatggcaagtgtttgctctttgccgcttggatctttccgatgatagtggctgggctgggttggggagattggtggggtggacttgtctatgctggcatcatacgtgcctgctttgttcagcaagccactttctgtgtcaactcattggcacattggataggtgaacaaccgtttgatgacagacgtactccaagggatcatgttctgactgcgttggtcacaatgggagaaggataccacaacttccaccatgagtttccgagtgactacagaaatgccatcatttggtatcagtatgaccctacaaagtggctcatctatctcttcagcttgggtcccttcccattggcctactctctcaagaccttccgttccaatgagattgagaaaggaaggcttcagcaacagcaaaaggctcttgacaagaaaagaagtggtcttgattggggacttcctctcttccagcttccagtgatctcatgggatgactttcaagctcgttgcaaagaaagtggagagatgcttgttgctgttgctggagtgatccatgatgtctcccagttcattgaagatcatcctggtgggaggagcctcattagaagtgctgttgggaaagatgggactggcatgttcaatggtggagtgtatgaacattcaaacgccgcacacaacttgctgagcacaatgagagttggagtcttgagaggtggacaagaagtggaggtttggaagaaacagagggtggatgttcttgggaagtcagacattcttcgtcaagtgacaagggtggagcgtctggtggaaggagctgttgcagcgtgatgagtagttagcttaatcacctagagctcggtcacctcgagtatcaaaatctatttagaaatacacaatattttgttgcaggcttgctggagaatcgatctgctatcataaaaattacaaaaaaattttatttgcctcaattattttaggattggtattaaggacgcttaaattatttgtcgggtcactacgcatcattgtgattgagaagatcagcgatacgaaatattcgtagtactatcgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaaggttggcgcgccgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgcggccgcactaggtttaaactctagaagctagg

SEQ ID NO: 56 - DNA, 9956 bp, Artificial
  Plasmid pDAB7323
ctagtctcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataatgatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatgatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataagatttatggtggactaattttcatatatttcttattgcttttaccttttcttggtatgtaagtccgtaactggaattactgtgggttgccatgacactctgtggtcttttggttcatgcatggatcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctaggtaccctgcagggatccaacaatggctcccaacatttctgaggatgtcaatggtgttctttttgagtcagatgcggcaacccctgatttggctctttccacaccacctgtgcaaaaagctgacaacagacccaagcaacttgtgtggaggaacattttgcttttcgcttacttgcacctcgcagctctctacggaggctatttgtttctcttcagtgcaaaatggcagaccgacattttcgcttacattctttatgtcatctctggactggggataactgctggggcacatagactctgggctcacaagtcatacaaagccaagtggccactcagagttatactggtcatcttcaacacggttgcctttcaagacgctgctatggattgggctcgtgaccatagaatgcatcacaagtacagcgagaccgacgcggacccacacaatgcaacgagaggtttcttcttctctcacattggctggcttcttgttaggaaacatcctgatctgaaagaaaaagggaagggactcgacatgagtgatctccttgctgatccaatactccgttttcagaagaagtactatctgatcctcatgcctctggcctgttttgtgatgccaaccgttatcccggtttacttttggggagaaacttggacaaatgctttcttcgtggcagccatgttccgttatgctttcatcctgaatgttacctggttggtgaactctgccgcacacaagtggggagacaaaccctatgacaagtccatcaagccttccgaaaacctttcagttgcgatgtttgctttgggagaaggatttcacaattaccatcacacttttccgtgggactacaagacagcagagcttggaaacaacaagttgaacttcacaacaacgttcatcaatttctttgcgaaaatcggttgggcctatgatttgaagactgtgagtgatgacattgtcaagaacagggtcaagagaactggcgatggaagccatcatctctggggctggggtgatgagaatcagagcaaagaagagatagatgcagccattaggatcaaccctaaagacgattgagtagttagcttaatcacctagagctcggtcacctcgagtatcaaaatctatttagaaatacacaatattttgttgcaggcttgctggagaatcgatctgctatcataaaaattacaaaaaaattttatttgcctcaattattttaggattggtattaaggacgcttaaattatttgtcgggtcactacgcatcattgtgattgagaagatcagcgatacgaaatattcgtagtactatcgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaaggttggcgcgccgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgcggccgcactaggtttaaactctagaagctaggaattcaaacaaagaagcgatcgcgcggccgccattgtactcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataatgatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatgatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataagatttatggtggactaattttcatatatttcttattgcttttaccttttcttggtatgtaagtccgtaactggaattactgtgggttgccatgacactctgtggtcttttggttcatgcatggatgcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctaggatccaacaatgtctgctccaaccgctgacatcagggctagggctccagaggctaagaaggttcacatcgctgataccgctatcaacaggcacaattggtacaagcacgtgaactggctcaacgtcttcctcatcatcggaatcccactctacggatgcatccaagctttctgggttccacttcaactcaagaccgctatctgggctgtgatctactacttcttcaccggacttggaatcaccgctggataccacaggctttgggctcactgctcatactctgctactcttccacttaggatctggcttgctgctgttggaggaggagctgttgagggatctatcagatggtgggctagggatcacagggctcatcataggtacaccgataccgacaaggacccatactctgttaggaagggacttctctactctcaccttggatggatggtgatgaagcagaacccaaagaggatcggaaggaccgacatctctgatctcaacgaggacccagttgttgtttggcaacacaggaactacctcaaggttgtgttcaccatgggacttgctgttccaatgcttgttgctggacttggatggggagattggcttggaggattcgtgtacgctggaatccttaggatcttcttcgttcaacaagctaccttctgcgtgaactctcttgctcactggcttggagatcaaccattcgatgataggaactctcctagggatcacgtgatcaccgctcttgttacccttggagagggataccacaacttccaccacgagttcccatctgactacaggaacgctatcgagtggcaccagtacgatcctaccaagtggtctatctgggcttggaagcaacttggattggcttacgatctcaagaagttcagggctaacgagatcgagaagggaagggttcaacaacttcagaagaagcttgataggaagagggctactcttgattggggaaccccacttgatcaacttccagtgatggaatgggatgactacgttgagcaagctaagaacggaaggggacttgttgctatcgctggagttgttcacgatgttaccgacttcatcaaggatcacccaggaggaaaggctatgatctcttctggaatcggaaaggatgctaccgctatgttcaacggaggagtgtactaccactctaacgcagctcacaaccttcttagcaccatgagggtgggagtgatcaggggaggatgcgaggttgagatctggaagagggctcagaaggagaacgttgagtacgttagggatggatctggacaaagggtgatcagggctggagagcaaccaaccaagatcccagagccaatcccaaccgctgatgctgcttgagtagttagcttaatcacctaggtcaccagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaactttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaattttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacccttatacaaaaccccaaaaacaagtttcctagcaccctaccaacgaattcgcggccgctttcctgcatgacatcgtcctgcagagccaagcgcatgcttaattaaa

SEQ ID NO: 57 - DNA, 5767 bp, Artificial
  Plasmid pDAB7325
aattcgcggccgctttcctgcatgacatcgtcctgcagagccaagcgcatgcttaattaaactagtctcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataatgatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatgatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataagatttatggtggactaattttcatatatttcttattgcttttaccttttcttggtatgtaagtccgtaactggaattactgtgggttgccatgacactctgtggtcttttggttcatgcatggatcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctaggtaccctgcagggatccaacaatggctcccaacatttctgaggatgtcaatggtgttctttttgagtcagatgcggcaacccctgatttggctctttccacaccacctgtgcaaaaagctgacaacagacccaagcaacttgtgtggaggaacattttgcttttcgcttacttgcacctcgcagctctctacggaggctatttgtttctcttcagtgcaaaatggcagaccgacattttcgcttacattctttatgtcatctctggactggggataactgctggggcacatagactctgggctcacaagtcatacaaagccaagtggccactcagagttatactggtcatcttcaacacggttgcctttcaagacgctgctatggattgggctcgtgaccatagaatgcatcacaagtacagcgagaccgacgcggacccacacaatgcaacgagaggtttcttcttctctcacattggctggcttcttgttaggaaacatcctgatctgaaagaaaaagggaagggactcgacatgagtgatctccttgctgatccaatactccgttttcagaagaagtactatctgatcctcatgcctctggcctgttttgtgatgccaaccgttatcccggtttacttttggggagaaacttggacaaatgctttcttcgtggcagccatgttccgttatgctttcatcctgaatgttacctggttggtgaactctgccgcacacaagtggggagacaaaccctatgacaagtccatcaagccttccgaaaacctttcagttgcgatgtttgctttgggagaaggatttcacaattaccatcacacttttccgtgggactacaagacagcagagcttggaaacaacaagttgaacttcacaacaacgttcatcaatttctttgcgaaaatcggttgggcctatgatttgaagactgtgagtgatgacattgtcaagaacagggtcaagagaactggcgatggaagccatcatctctggggctggggtgatgagaatcagagcaaagaagagatagatgcagccattaggatcaaccctaaagacgattgagtagttagcttaatcacctagagctcggtcacctcgagtatcaaaatctatttagaaatacacaatattttgttgcaggcttgctggagaatcgatctgctatcataaaaattacaaaaaaattttatttgcctcaattattttaggattggtattaaggacgcttaaattatttgtcgggtcactacgcatcattgtgattgagaagatcagcgatacgaaatattcgtagtactatcgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaaggttggcgcgccgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgcggccgcactaggtttaaactctagaagctagg

SEQ ID NO: 58 - DNA, 6109 bp, Artificial
  Plasmid pDAB7327
cggtcacctcgagtatcaaaatctatttagaaatacacaatattttgttgcaggcttgctggagaatcgatctgctatcataaaaattacaaaaaaattttatttgcctcaattattttaggattggtattaaggacgcttaaattatttgtcgggtcactacgcatcattgtgattgagaagatcagcgatacgaaatattcgtagtactatcgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaaggttggcgcgccgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgcggccgcactaggtttaaactctagaagctaggaattcgcggccgctttcctgcatgacatcgtcctgcagagccaagcgcatgcttaattaaactagtctcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataatgatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatgatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataagatttatggtggactaattttcatatatttcttattgcttttaccttttcttggtatgtaagtccgtaactggaattactgtgggttgccatgacactctgtggtcttttggttcatgcatggatcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctaggtaccctgcagggatccaacaatgtctgctccaaccgctgacatcagggctagggctccagaggctaagaaggttcacatcgctgataccgctatcaacaggcacaattggtacaagcacgtgaactggctcaacgtcttcctcatcatcggaatcccactctacggatgcatccaagctttctgggttccacttcaactcaagaccgctatctgggctgtgatctactacttcttcaccggacttggaatcaccgctggataccacaggctttgggctcactgctcatactctgctactcttccacttaggatctggcttgctgctgttggaggaggagctgttgagggatctatcagatggtgggctagggatcacagggctcatcataggtacaccgataccgacaaggacccatactctgttaggaagggacttctctactctcaccttggatggatggtgatgaagcagaacccaaagaggatcggaaggaccgacatctctgatctcaacgaggacccagttgttgtttggcaacacaggaactacctcaaggttgtgttcaccatgggacttgctgttccaatgcttgttgctggacttggatggggagattggcttggaggattcgtgtacgctggaatccttaggatcttcttcgttcaacaagctaccttctgcgtgaactctcttgctcactggcttggagatcaaccattcgatgataggaactctcctagggatcacgtgatcaccgctcttgttacccttggagagggataccacaacttccaccacgagttcccatctgactacaggaacgctatcgagtggcaccagtacgatcctaccaagtggtctatctgggcttggaagcaacttggattggcttacgatctcaagaagttcagggctaacgagatcgagaagggaagggttcaacaacttcagaagaagcttgataggaagagggctactcttgattggggaaccccacttgatcaacttccagtgatggaatgggatgactacgttgagcaagctaagaacggaaggggacttgttgctatcgctggagttgttcacgatgttaccgacttcatcaaggatcacccaggaggaaaggctatgatctcttctggaatcggaaaggatgctaccgctatgttcaacggaggagtgtactaccactctaacgcagctcacaaccttcttagcaccatgagggtgggagtgatcaggggaggatgcgaggttgagatctggaagagggctcagaaggagaacgttgagtacgttagggatggatctggacaaagggtgatcagggctggagagcaaccaaccaagatcccagagccaatcccaaccgctgatgctgcttgagtagttagcttaatcacctaggtcaccagtatgaactaaaatgcatgtaggtgtaagagct

SEQ ID NO: 59 - DNA, 6136 bp, Artificial
  Plasmid pDAB7329
cggtcacctcgagtatcaaaatctatttagaaatacacaatattttgttgcaggcttgctggagaatcgatctgctatcataaaaattacaaaaaaattttatttgcctcaattattttaggattggtattaaggacgcttaaattatttgtcgggtcactacgcatcattgtgattgagaagatcagcgatacgaaatattcgtagtactatcgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaaggttggcgcgccgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgcggccgcactaggtttaaactctagaagctaggaattcgcggccgctttcctgcatgacatcgtcctgcagagccaagcgcatgcttaattaaactagtctcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataatgatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatgatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataagatttatggtggactaattttcatatatttcttattgcttttaccttttcttggtatgtaagtccgtaactggaattactgtgggttgccatgacactctgtggtcttttggttcatgcatggatcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctaggtaccctgcagggatccaacaatggccagcagttcttcaagtgtgccagaacttgccgcagctttccctgatgggacaacggacttcaaacccatgaggaacaccaaaggctatgatgtctccaaacctcacatctctgaaacaccgatgactttgaagaactggcacaaacatgtgaactggctcaacaccacattcattctctttgttccactggctgggttgatctcaacctattgggttcctcttcaatggaaaactgcagtgtgggcagttgtgtactacttcaacactggacttgggatcactgctggctaccatagattgtgggcacattcctcttacaaggccagcttgcctctcaaaatctaccttgccgcagttggtgctggagccgttgaaggttccataagatggtggagcaacggacacagagcacatcacagatacacagacacagagaaagatccttactcagtgaggaagggattgctctacagccacatgggttggatgctcttgaagcagaatccaaagaagcaagggaggacggacattactgatctgaatgaggacccagttgtggtctggcaacataggaactttctcaagtgtgtgatcttcatggctttggtctttcccacccttgttgctggcctgggatggggagactactggggaggtttcatctatggagggatcttgagagtgttctttgttcagcaagccaccttctgtgtcaactcacttgcacattggcttggtgatcaaccgtttgatgacagaaactctccacgtgaccatgtcataactgctcttgtcacgctgggtgaaggctatcacaactttcaccatgagtttccgtcagactatagaaatgcgattgagtggtatcagtatgaccccacgaagtggagcatttggatttggaagcaacttggacttgctcacaatctcaagcagttcagacagaatgagatagagaagggaagggttcaacagttgcagaagaaactggatcagaagagagcgaaacttgattggggaataccgttggaacaactccctgttgtgtcttgggatgactttgttgaacagtcaaagaatggcaaggcatggattgctgttgctggtgtcattcacgatgttggtgacttcatcaaggatcatcctggtggacgtgctctcatcaactctgcgattggcaaagatgccacagcgatcttcaatggaggtgtctacaatcattcaaatgccgcacacaaccttctctccaccatgagggttggtgtcctccgtggagggtgcgaagtggagatatggaaacgtgctcaaagtgagaacaaagatgtctctactgtggttgatagttctggcaaccgtattgtgagagctggtggacaagctaccaaagtggttcagccagtccctggtgctcaagcagcttgatgagtagttagcttaatcacctagagct

SEQ ID NO: 60 - DNA, 18713 bp, Artificial
  Plasmid pDAB7319
ttgtacaaagtggttgcggccgcttaattaaatttaaattcaattaatgcaatcttgattttcaacaacgaaggtaatggcgtaaaagaaaaaatgtatgttattgtattgatctttcatgatgttgaagcgtgccataatatgatgatgtataattaaaatattaactgtcgcattttattgaaatggcactgttatttcaaccatatctttgattctgttacatgacacgactgcaagaagtaaataatagacgccgttgttaaagaattgctatcatatgtgcctaactagagggaatttgagcgtcagacctaatcaaatattacaaaatatctcactctgtcgccagcaatggtgtaatcagcgcagacaaatggcgtaaagatcgcggaaaaacctccccgagtggcatgatagctgcctctgtattgctgatttagtcagccttatttgacttaagggtgccctcgttagtgacaaattgctttcaaggagacagccatgccccacactttgttgaaaaacaaattgcctttggggagacggtaaagccagttgctcttcaataaggaatgtcgaggaggcaatgtaaccgcctctggtagtacacttctctaatccaaaaatcaatttgtattcaagataccgcaaaaaacttatggtttaaaccctgcaggactagtccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtaggtaccagatctggatcccaaaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatctacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccaaatctgagtagttagcttaatcacctagagctcgatcggcggcaatagcttcttagcgccatcccgggttgatcctatctgtgttgaaatagttgcggtgggcaaggctctctttcagaaagacaggcggccaaaggaacccaaggtgaggtgggctatggctctcagttccttgtggaagcgcttggtctaaggtgcagaggtgttagcgggatgaagcaaaagtgtccgattgtaacaagatatgttgatcctacgtaaggatattaaagtatgtattcatcactaatataatcagtgtattccaatatgtactacgatttccaatgtctttattgtcgccgtatgtaatcggcgtcacaaaataatccccggtgactttcttttaatccaggatgaaataatatgttattataatttttgcgatttggtccgttataggaattgaagtgtgcttgaggtcggtcgccaccactcccatttcataattttacatgtatttgaaaaataaaaatttatggtattcaatttaaacacgtatacttgtaaagaatgatatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatggtgtaatacataggaattcgtttaaacgatctgcgtctaattttcggtccaacttgcacaggaaagacgtcgaccgcggtagctcttgcccagcagactgggcttccagtcctttcgctcgatcgggtccaatgttgtcctcagctgtgaaccggaagcggacgaccaacagtggaagaactgaaaggaacgagccgtctataccttgatgatcggcctctggtgaagggtatcatcgcagccaagcaagctcatgaaaggctgatgggggaggtgtataattatgaggcccacggcgggcttattctttagggaggatctatctcgttgctcaagtgcatggcgcaaagcagttattggagtgcggattttcgttggcatattattcgccacgagttagcagacgaagagaccttcatgaacgtggccaaggccagagttaagcagatgttacgccctgctgcaggcctttctattatccaatagttggttgatctttggaaagagcctcggctgaggcccatactgaaagagatcgatggatatcgatatgccatgttgtttgctagccagaaccagatcacatccgatatgctattgcagcttgacgcagatatggaggataagttgattcatgggatcgctcaggagtagctcatccatgcacgccgacaagaacagaaattccgtcgagttaacgcagccgcttacgacggattcgaaggtcatccattcggaatgtattagtttgcaccagctccgcgtcacacctgtcttcatttgaataagatgttagcaattgtttttagctttgtcttgttgtggcagggcggcaagtgcttcagacatcattctgttttcaaattttatgctggagaacagcttcttaattcctttggaaataatagactgcgtcttaaaattcagatgtctggatatagatatgattgtaaaataacctatttaagtgtcatttagaacataagttttatgaatgttcttccattttcgtcatcgaacgaataagagtaaatacaccttttttaacattacaaataagttcttatacgttgtttatacaccgggaatcatttccattattttcgcgcaaaagtcacggatattcgtgaaagcgacataaactgcgaaatttgcggggagtgtcttgagtttgcctcgaggctagcgcatgcacatagacacacacatcatctcattgatgcttggtaataattgtcattagattgtttttatgcatagatgcactcgaaatcagccaattttagacaagtatcaaacggatgtgacttcagtacattaaaaacgtccgcaatgtgttattaagttgtctaagcgtcaatttgatttacaattgaatatatcctgccccagccagccaacagctcgatttacaattgaatatatcctgccggccggcccacgcgtgtcgaggaattctgatctggcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttaaacatcatccgtcggatggcgttgcgagagaagcagtcgatccgtgagatcagccgacgcaccgggcaggcgcgcaacacgatcgcaaagtatttgaacgcaggtacaatcgagccgacgttcacgcggaacgaccaagcaagcttggctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaagggtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagcccccgagcgcttagtgggaatttgtaccccttatcgaaccgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtcttgcgatggcatttcgatccattcccattccgcgctcaagatggcttcccctcggcagttcatcagggctaaatcaatctagccgacttgtccggtgaaatgggctgcactccaacagaaacaatcaaacaaacatacacagcgacttattcacacgagctcaaattacaacggtatatatcctgccagtcagcatcatcacaccaaaagttaggcccgaatagtttgaaattagaaagctcgcaattgaggtctacaggccaaattcgctcttagccgtacaatattactcaccggatcctaaccggtgtgatcatgggccgcgattaaaaatctcaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaacctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaatgaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggccaccgcttaattaaggcgcgccatgcccgggcaagcggccgcacaagtttgtacaaaaaagcaggctccgcggccgcactaggtttaaactctagaagctaggaattcaaacaaagaagcgatcgcgcggccgccattgtactcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataatgatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatgatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataagatttatggtggactaattttcatatatttcttattgcttttaccttttcttggtatgtaagtccgtaactggaattactgtgggttgccatgacactctgtggtcttttggttcatgcatggatgcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctaggatccaacaatgtctgctccaaccgctgacatcagggctagggctccagaggctaagaaggttcacatcgctgataccgctatcaacaggcacaattggtacaagcacgtgaactggctcaacgtcttcctcatcatcggaatcccactctacggatgcatccaagctttctgggttccacttcaactcaagaccgctatctgggctgtgatctactacttcttcaccggacttggaatcaccgctggataccacaggctttgggctcactgctcatactctgctactcttccacttaggatctggcttgctgctgttggaggaggagctgttgagggatctatcagatggtgggctagggatcacagggctcatcataggtacaccgataccgacaaggacccatactctgttaggaagggacttctctactctcaccttggatggatggtgatgaagcagaacccaaagaggatcggaaggaccgacatctctgatctcaacgaggacccagttgttgtttggcaacacaggaactacctcaaggttgtgttcaccatgggacttgctgttccaatgcttgttgctggacttggatggggagattggcttggaggattcgtgtacgctggaatccttaggatcttcttcgttcaacaagctaccttctgcgtgaactctcttgctcactggcttggagatcaaccattcgatgataggaactctcctagggatcacgtgatcaccgctcttgttacccttggagagggataccacaacttccaccacgagttcccatctgactacaggaacgctatcgagtggcaccagtacgatcctaccaagtggtctatctgggcttggaagcaacttggattggcttacgatctcaagaagttcagggctaacgagatcgagaagggaagggttcaacaacttcagaagaagcttgataggaagagggctactcttgattggggaaccccacttgatcaacttccagtgatggaatgggatgactacgttgagcaagctaagaacggaaggggacttgttgctatcgctggagttgttcacgatgttaccgacttcatcaaggatcacccaggaggaaaggctatgatctcttctggaatcggaaaggatgctaccgctatgttcaacggaggagtgtactaccactctaacgcagctcacaaccttcttagcaccatgagggtgggagtgatcaggggaggatgcgaggttgagatctggaagagggctcagaaggagaacgttgagtacgttagggatggatctggacaaagggtgatcagggctggagagcaaccaaccaagatcccagagccaatcccaaccgctgatgctgcttgagtagttagcttaatcacctaggtcaccagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaactttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaattttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacccttatacaaaaccccaaaaacaagtttcctagcaccctaccaacgaattcgcggccgctttcctgcatgacatcgtcctgcagagccaagcgcatgcttaattaaactagtctcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataatgatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatgatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataagatttatggtggactaattttcatatatttcttattgcttttaccttttcttggtatgtaagtccgtaactggaattactgtgggttgccatgacactctgtggtcttttggttcatgcatggatcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctaggtaccctgcagggatccaacaatggctgcacttgatagcatccctgaggacaaagcaactagctccaagtcaacccacatacagtaccaagaggtcacgtttaggaactggtacaagaaaatcaactggctcaacacgacccttgttgtcctcattcctgctcttgggttgtacttgacgagaaccacacctctcaccagacctaccctcatttggtctgttctctactatttctgtacagcgtttggcatcactggtggctaccacagactttggtcccataggtcttacagtgcgaggttgccattgagactcttcctggctttcactggagctggtgcgatccaaggttctgcaagatggtggtcagccaatcatagggcacatcaccgttggacggacaccatgaaggacccctactctgtgatgagaggactgctgttctcccacataggttggatggttctcaactctgatccaaaggtcaaaggcagaacagatgtttctgatcttgactctgatcccgtcgttgtgtggcaacacaaacactatggcaagtgtttgctctttgccgcttggatctttccgatgatagtggctgggctgggttggggagattggtggggtggacttgtctatgctggcatcatacgtgcctgctttgttcagcaagccactttctgtgtcaactcattggcacattggataggtgaacaaccgtttgatgacagacgtactccaagggatcatgttctgactgcgttggtcacaatgggagaaggataccacaacttccaccatgagtttccgagtgactacagaaatgccatcatttggtatcagtatgaccctacaaagtggctcatctatctcttcagcttgggtcccttcccattggcctactctctcaagaccttccgttccaatgagattgagaaaggaaggcttcagcaacagcaaaaggctcttgacaagaaaagaagtggtcttgattggggacttcctctcttccagcttccagtgatctcatgggatgactttcaagctcgttgcaaagaaagtggagagatgcttgttgctgttgctggagtgatccatgatgtctcccagttcattgaagatcatcctggtgggaggagcctcattagaagtgctgttgggaaagatgggactggcatgttcaatggtggagtgtatgaacattcaaacgccgcacacaacttgctgagcacaatgagagttggagtcttgagaggtggacaagaagtggaggtttggaagaaacagagggtggatgttcttgggaagtcagacattcttcgtcaagtgacaagggtggagcgtctggtggaaggagctgttgcagcgtgatgagtagttagcttaatcacctagagctcggtcacctcgagtatcaaaatctatttagaaatacacaatattttgttgcaggcttgctggagaatcgatctgctatcataaaaattacaaaaaaattttatttgcctcaattattttaggattggtattaaggacgcttaaattatttgtcgggtcactacgcatcattgtgattgagaagatcagcgatacgaaatattcgtagtactatcgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaaggttggcgcgccgacccagctttc

SEQ ID NO: 61 - DNA, 14524 bp, Artificial
  Plasmid pDAB7321
ttgtacaaagtggttgcggccgcttaattaaatttaaattcaattaatgcaatcttgattttcaacaacgaaggtaatggcgtaaaagaaaaaatgtatgttattgtattgatctttcatgatgttgaagcgtgccataatatgatgatgtataattaaaatattaactgtcgcattttattgaaatggcactgttatttcaaccatatctttgattctgttacatgacacgactgcaagaagtaaataatagacgccgttgttaaagaattgctatcatatgtgcctaactagagggaatttgagcgtcagacctaatcaaatattacaaaatatctcactctgtcgccagcaatggtgtaatcagcgcagacaaatggcgtaaagatcgcggaaaaacctccccgagtggcatgatagctgcctctgtattgctgatttagtcagccttatttgacttaagggtgccctcgttagtgacaaattgctttcaaggagacagccatgccccacactttgttgaaaaacaaattgcctttggggagacggtaaagccagttgctcttcaataaggaatgtcgaggaggcaatgtaaccgcctctggtagtacacttctctaatccaaaaatcaatttgtattcaagataccgcaaaaaacttatggtttaaaccctgcaggactagtccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtaggtaccagatctggatcccaaaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatctacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccaaatctgagtagttagcttaatcacctagagctcgatcggcggcaatagcttcttagcgccatcccgggttgatcctatctgtgttgaaatagttgcggtgggcaaggctctctttcagaaagacaggcggccaaaggaacccaaggtgaggtgggctatggctctcagttccttgtggaagcgcttggtctaaggtgcagaggtgttagcgggatgaagcaaaagtgtccgattgtaacaagatatgttgatcctacgtaaggatattaaagtatgtattcatcactaatataatcagtgtattccaatatgtactacgatttccaatgtctttattgtcgccgtatgtaatcggcgtcacaaaataatccccggtgactttcttttaatccaggatgaaataatatgttattataatttttgcgatttggtccgttataggaattgaagtgtgcttgaggtcggtcgccaccactcccatttcataattttacatgtatttgaaaaataaaaatttatggtattcaatttaaacacgtatacttgtaaagaatgatatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatggtgtaatacataggaattcgtttaaacgatctgcgtctaattttcggtccaacttgcacaggaaagacgtcgaccgcggtagctcttgcccagcagactgggcttccagtcctttcgctcgatcgggtccaatgttgtcctcagctgtgaaccggaagcggacgaccaacagtggaagaactgaaaggaacgagccgtctataccttgatgatcggcctctggtgaagggtatcatcgcagccaagcaagctcatgaaaggctgatgggggaggtgtataattatgaggcccacggcgggcttattctttagggaggatctatctcgttgctcaagtgcatggcgcaaagcagttattggagtgcggattttcgttggcatattattcgccacgagttagcagacgaagagaccttcatgaacgtggccaaggccagagttaagcagatgttacgccctgctgcaggcctttctattatccaatagttggttgatctttggaaagagcctcggctgaggcccatactgaaagagatcgatggatatcgatatgccatgttgtttgctagccagaaccagatcacatccgatatgctattgcagcttgacgcagatatggaggataagttgattcatgggatcgctcaggagtagctcatccatgcacgccgacaagaacagaaattccgtcgagttaacgcagccgcttacgacggattcgaaggtcatccattcggaatgtattagtttgcaccagctccgcgtcacacctgtcttcatttgaataagatgttagcaattgtttttagctttgtcttgttgtggcagggcggcaagtgcttcagacatcattctgttttcaaattttatgctggagaacagcttcttaattcctttggaaataatagactgcgtcttaaaattcagatgtctggatatagatatgattgtaaaataacctatttaagtgtcatttagaacataagttttatgaatgttcttccattttcgtcatcgaacgaataagagtaaatacaccttttttaacattacaaataagttcttatacgttgtttatacaccgggaatcatttccattattttcgcgcaaaagtcacggatattcgtgaaagcgacataaactgcgaaatttgcggggagtgtcttgagtttgcctcgaggctagcgcatgcacatagacacacacatcatctcattgatgcttggtaataattgtcattagattgtttttatgcatagatgcactcgaaatcagccaattttagacaagtatcaaacggatgtgacttcagtacattaaaaacgtccgcaatgtgttattaagttgtctaagcgtcaatttgatttacaattgaatatatcctgccccagccagccaacagctcgatttacaattgaatatatcctgccggccggcccacgcgtgtcgaggaattctgatctggcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttaaacatcatccgtcggatggcgttgcgagagaagcagtcgatccgtgagatcagccgacgcaccgggcaggcgcgcaacacgatcgcaaagtatttgaacgcaggtacaatcgagccgacgttcacgcggaacgaccaagcaagcttggctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaagggtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagcccccgagcgcttagtgggaatttgtaccccttatcgaaccgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtcttgcgatggcatttcgatccattcccattccgcgctcaagatggcttcccctcggcagttcatcagggctaaatcaatctagccgacttgtccggtgaaatgggctgcactccaacagaaacaatcaaacaaacatacacagcgacttattcacacgagctcaaattacaacggtatatatcctgccagtcagcatcatcacaccaaaagttaggcccgaatagtttgaaattagaaagctcgcaattgaggtctacaggccaaattcgctcttagccgtacaatattactcaccggatcctaaccggtgtgatcatgggccgcgattaaaaatctcaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaacctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaatgaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggccaccgcttaattaaggcgcgccatgcccgggcaagcggccgcacaagtttgtacaaaaaagcaggctccgcggccgcactaggtttaaactctagaagctaggaattcgcggccgctttcctgcatgacatcgtcctgcagagccaagcgcatgcttaattaaactagtctcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataatgatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatgatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataagatttatggtggactaattttcatatatttcttattgcttttaccttttcttggtatgtaagtccgtaactggaattactgtgggttgccatgacactctgtggtcttttggttcatgcatggatcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctaggtaccctgcagggatccaacaatggctgcacttgatagcatccctgaggacaaagcaactagctccaagtcaacccacatacagtaccaagaggtcacgtttaggaactggtacaagaaaatcaactggctcaacacgacccttgttgtcctcattcctgctcttgggttgtacttgacgagaaccacacctctcaccagacctaccctcatttggtctgttctctactatttctgtacagcgtttggcatcactggtggctaccacagactttggtcccataggtcttacagtgcgaggttgccattgagactcttcctggctttcactggagctggtgcgatccaaggttctgcaagatggtggtcagccaatcatagggcacatcaccgttggacggacaccatgaaggacccctactctgtgatgagaggactgctgttctcccacataggttggatggttctcaactctgatccaaaggtcaaaggcagaacagatgtttctgatcttgactctgatcccgtcgttgtgtggcaacacaaacactatggcaagtgtttgctctttgccgcttggatctttccgatgatagtggctgggctgggttggggagattggtggggtggacttgtctatgctggcatcatacgtgcctgctttgttcagcaagccactttctgtgtcaactcattggcacattggataggtgaacaaccgtttgatgacagacgtactccaagggatcatgttctgactgcgttggtcacaatgggagaaggataccacaacttccaccatgagtttccgagtgactacagaaatgccatcatttggtatcagtatgaccctacaaagtggctcatctatctcttcagcttgggtcccttcccattggcctactctctcaagaccttccgttccaatgagattgagaaaggaaggcttcagcaacagcaaaaggctcttgacaagaaaagaagtggtcttgattggggacttcctctcttccagcttccagtgatctcatgggatgactttcaagctcgttgcaaagaaagtggagagatgcttgttgctgttgctggagtgatccatgatgtctcccagttcattgaagatcatcctggtgggaggagcctcattagaagtgctgttgggaaagatgggactggcatgttcaatggtggagtgtatgaacattcaaacgccgcacacaacttgctgagcacaatgagagttggagtcttgagaggtggacaagaagtggaggtttggaagaaacagagggtggatgttcttgggaagtcagacattcttcgtcaagtgacaagggtggagcgtctggtggaaggagctgttgcagcgtgatgagtagttagcttaatcacctagagctcggtcacctcgagtatcaaaatctatttagaaatacacaatattttgttgcaggcttgctggagaatcgatctgctatcataaaaattacaaaaaaattttatttgcctcaattattttaggattggtattaaggacgcttaaattatttgtcgggtcactacgcatcattgtgattgagaagatcagcgatacgaaatattcgtagtactatcgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaaggttggcgcgccgacccagctttc

SEQ ID NO: 62 - DNA, 18422 bp, Artificial
  Plasmid pDAB7324
ttgtacaaagtggttgcggccgcttaattaaatttaaattcaattaatgcaatcttgattttcaacaacgaaggtaatggcgtaaaagaaaaaatgtatgttattgtattgatctttcatgatgttgaagcgtgccataatatgatgatgtataattaaaatattaactgtcgcattttattgaaatggcactgttatttcaaccatatctttgattctgttacatgacacgactgcaagaagtaaataatagacgccgttgttaaagaattgctatcatatgtgcctaactagagggaatttgagcgtcagacctaatcaaatattacaaaatatctcactctgtcgccagcaatggtgtaatcagcgcagacaaatggcgtaaagatcgcggaaaaacctccccgagtggcatgatagctgcctctgtattgctgatttagtcagccttatttgacttaagggtgccctcgttagtgacaaattgctttcaaggagacagccatgccccacactttgttgaaaaacaaattgcctttggggagacggtaaagccagttgctcttcaataaggaatgtcgaggaggcaatgtaaccgcctctggtagtacacttctctaatccaaaaatcaatttgtattcaagataccgcaaaaaacttatggtttaaaccctgcaggactagtccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtaggtaccagatctggatcccaaaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatctacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccaaatctgagtagttagcttaatcacctagagctcgatcggcggcaatagcttcttagcgccatcccgggttgatcctatctgtgttgaaatagttgcggtgggcaaggctctctttcagaaagacaggcggccaaaggaacccaaggtgaggtgggctatggctctcagttccttgtggaagcgcttggtctaaggtgcagaggtgttagcgggatgaagcaaaagtgtccgattgtaacaagatatgttgatcctacgtaaggatattaaagtatgtattcatcactaatataatcagtgtattccaatatgtactacgatttccaatgtctttattgtcgccgtatgtaatcggcgtcacaaaataatccccggtgactttcttttaatccaggatgaaataatatgttattataatttttgcgatttggtccgttataggaattgaagtgtgcttgaggtcggtcgccaccactcccatttcataattttacatgtatttgaaaaataaaaatttatggtattcaatttaaacacgtatacttgtaaagaatgatatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatggtgtaatacataggaattcgtttaaacgatctgcgtctaattttcggtccaacttgcacaggaaagacgtcgaccgcggtagctcttgcccagcagactgggcttccagtcctttcgctcgatcgggtccaatgttgtcctcagctgtgaaccggaagcggacgaccaacagtggaagaactgaaaggaacgagccgtctataccttgatgatcggcctctggtgaagggtatcatcgcagccaagcaagctcatgaaaggctgatgggggaggtgtataattatgaggcccacggcgggcttattctttagggaggatctatctcgttgctcaagtgcatggcgcaaagcagttattggagtgcggattttcgttggcatattattcgccacgagttagcagacgaagagaccttcatgaacgtggccaaggccagagttaagcagatgttacgccctgctgcaggcctttctattatccaatagttggttgatctttggaaagagcctcggctgaggcccatactgaaagagatcgatggatatcgatatgccatgttgtttgctagccagaaccagatcacatccgatatgctattgcagcttgacgcagatatggaggataagttgattcatgggatcgctcaggagtagctcatccatgcacgccgacaagaacagaaattccgtcgagttaacgcagccgcttacgacggattcgaaggtcatccattcggaatgtattagtttgcaccagctccgcgtcacacctgtcttcatttgaataagatgttagcaattgtttttagctttgtcttgttgtggcagggcggcaagtgcttcagacatcattctgttttcaaattttatgctggagaacagcttcttaattcctttggaaataatagactgcgtcttaaaattcagatgtctggatatagatatgattgtaaaataacctatttaagtgtcatttagaacataagttttatgaatgttcttccattttcgtcatcgaacgaataagagtaaatacaccttttttaacattacaaataagttcttatacgttgtttatacaccgggaatcatttccattattttcgcgcaaaagtcacggatattcgtgaaagcgacataaactgcgaaatttgcggggagtgtcttgagtttgcctcgaggctagcgcatgcacatagacacacacatcatctcattgatgcttggtaataattgtcattagattgtttttatgcatagatgcactcgaaatcagccaattttagacaagtatcaaacggatgtgacttcagtacattaaaaacgtccgcaatgtgttattaagttgtctaagcgtcaatttgatttacaattgaatatatcctgccccagccagccaacagctcgatttacaattgaatatatcctgccggccggcccacgcgtgtcgaggaattctgatctggcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttaaacatcatccgtcggatggcgttgcgagagaagcagtcgatccgtgagatcagccgacgcaccgggcaggcgcgcaacacgatcgcaaagtatttgaacgcaggtacaatcgagccgacgttcacgcggaacgaccaagcaagcttggctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaagggtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagcccccgagcgcttagtgggaatttgtaccccttatcgaaccgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtcttgcgatggcatttcgatccattcccattccgcgctcaagatggcttcccctcggcagttcatcagggctaaatcaatctagccgacttgtccggtgaaatgggctgcactccaacagaaacaatcaaacaaacatacacagcgacttattcacacgagctcaaattacaacggtatatatcctgccagtcagcatcatcacaccaaaagttaggcccgaatagtttgaaattagaaagctcgcaattgaggtctacaggccaaattcgctcttagccgtacaatattactcaccggatcctaaccggtgtgatcatgggccgcgattaaaaatctcaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaacctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaatgaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggccaccgcttaattaaggcgcgccatgcccgggcaagcggccgcacaagtttgtacaaaaaagcaggctccgcggccgcactaggtttaaactctagaagctaggaattcaaacaaagaagcgatcgcgcggccgccattgtactcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataatgatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatgatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataagatttatggtggactaattttcatatatttcttattgcttttaccttttcttggtatgtaagtccgtaactggaattactgtgggttgccatgacactctgtggtcttttggttcatgcatggatgcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctaggatccaacaatgtctgctccaaccgctgacatcagggctagggctccagaggctaagaaggttcacatcgctgataccgctatcaacaggcacaattggtacaagcacgtgaactggctcaacgtcttcctcatcatcggaatcccactctacggatgcatccaagctttctgggttccacttcaactcaagaccgctatctgggctgtgatctactacttcttcaccggacttggaatcaccgctggataccacaggctttgggctcactgctcatactctgctactcttccacttaggatctggcttgctgctgttggaggaggagctgttgagggatctatcagatggtgggctagggatcacagggctcatcataggtacaccgataccgacaaggacccatactctgttaggaagggacttctctactctcaccttggatggatggtgatgaagcagaacccaaagaggatcggaaggaccgacatctctgatctcaacgaggacccagttgttgtttggcaacacaggaactacctcaaggttgtgttcaccatgggacttgctgttccaatgcttgttgctggacttggatggggagattggcttggaggattcgtgtacgctggaatccttaggatcttcttcgttcaacaagctaccttctgcgtgaactctcttgctcactggcttggagatcaaccattcgatgataggaactctcctagggatcacgtgatcaccgctcttgttacccttggagagggataccacaacttccaccacgagttcccatctgactacaggaacgctatcgagtggcaccagtacgatcctaccaagtggtctatctgggcttggaagcaacttggattggcttacgatctcaagaagttcagggctaacgagatcgagaagggaagggttcaacaacttcagaagaagcttgataggaagagggctactcttgattggggaaccccacttgatcaacttccagtgatggaatgggatgactacgttgagcaagctaagaacggaaggggacttgttgctatcgctggagttgttcacgatgttaccgacttcatcaaggatcacccaggaggaaaggctatgatctcttctggaatcggaaaggatgctaccgctatgttcaacggaggagtgtactaccactctaacgcagctcacaaccttcttagcaccatgagggtgggagtgatcaggggaggatgcgaggttgagatctggaagagggctcagaaggagaacgttgagtacgttagggatggatctggacaaagggtgatcagggctggagagcaaccaaccaagatcccagagccaatcccaaccgctgatgctgcttgagtagttagcttaatcacctaggtcaccagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaactttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaattttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacccttatacaaaaccccaaaaacaagtttcctagcaccctaccaacgaattcgcggccgctttcctgcatgacatcgtcctgcagagccaagcgcatgcttaattaaactagtctcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataatgatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatgatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataagatttatggtggactaattttcatatatttcttattgcttttaccttttcttggtatgtaagtccgtaactggaattactgtgggttgccatgacactctgtggtcttttggttcatgcatggatcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctaggtaccctgcagggatccaacaatggctcccaacatttctgaggatgtcaatggtgttctttttgagtcagatgcggcaacccctgatttggctctttccacaccacctgtgcaaaaagctgacaacagacccaagcaacttgtgtggaggaacattttgcttttcgcttacttgcacctcgcagctctctacggaggctatttgtttctcttcagtgcaaaatggcagaccgacattttcgcttacattctttatgtcatctctggactggggataactgctggggcacatagactctgggctcacaagtcatacaaagccaagtggccactcagagttatactggtcatcttcaacacggttgcctttcaagacgctgctatggattgggctcgtgaccatagaatgcatcacaagtacagcgagaccgacgcggacccacacaatgcaacgagaggtttcttcttctctcacattggctggcttcttgttaggaaacatcctgatctgaaagaaaaagggaagggactcgacatgagtgatctccttgctgatccaatactccgttttcagaagaagtactatctgatcctcatgcctctggcctgttttgtgatgccaaccgttatcccggtttacttttggggagaaacttggacaaatgctttcttcgtggcagccatgttccgttatgctttcatcctgaatgttacctggttggtgaactctgccgcacacaagtggggagacaaaccctatgacaagtccatcaagccttccgaaaacctttcagttgcgatgtttgctttgggagaaggatttcacaattaccatcacacttttccgtgggactacaagacagcagagcttggaaacaacaagttgaacttcacaacaacgttcatcaatttctttgcgaaaatcggttgggcctatgatttgaagactgtgagtgatgacattgtcaagaacagggtcaagagaactggcgatggaagccatcatctctggggctggggtgatgagaatcagagcaaagaagagatagatgcagccattaggatcaaccctaaagacgattgagtagttagcttaatcacctagagctcggtcacctcgagtatcaaaatctatttagaaatacacaatattttgttgcaggcttgctggagaatcgatctgctatcataaaaattacaaaaaaattttatttgcctcaattattttaggattggtattaaggacgcttaaattatttgtcgggtcactacgcatcattgtgattgagaagatcagcgatacgaaatattcgtagtactatcgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaaggttggcgcgccgacccagctttc

SEQ ID NO: 63 - DNA, 14233 bp, Artificial
  Plasmid pDAB7326
ttgtacaaagtggttgcggccgcttaattaaatttaaattcaattaatgcaatcttgattttcaacaacgaaggtaatggcgtaaaagaaaaaatgtatgttattgtattgatctttcatgatgttgaagcgtgccataatatgatgatgtataattaaaatattaactgtcgcattttattgaaatggcactgttatttcaaccatatctttgattctgttacatgacacgactgcaagaagtaaataatagacgccgttgttaaagaattgctatcatatgtgcctaactagagggaatttgagcgtcagacctaatcaaatattacaaaatatctcactctgtcgccagcaatggtgtaatcagcgcagacaaatggcgtaaagatcgcggaaaaacctccccgagtggcatgatagctgcctctgtattgctgatttagtcagccttatttgacttaagggtgccctcgttagtgacaaattgctttcaaggagacagccatgccccacactttgttgaaaaacaaattgcctttggggagacggtaaagccagttgctcttcaataaggaatgtcgaggaggcaatgtaaccgcctctggtagtacacttctctaatccaaaaatcaatttgtattcaagataccgcaaaaaacttatggtttaaaccctgcaggactagtccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtaggtaccagatctggatcccaaaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatctacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccaaatctgagtagttagcttaatcacctagagctcgatcggcggcaatagcttcttagcgccatcccgggttgatcctatctgtgttgaaatagttgcggtgggcaaggctctctttcagaaagacaggcggccaaaggaacccaaggtgaggtgggctatggctctcagttccttgtggaagcgcttggtctaaggtgcagaggtgttagcgggatgaagcaaaagtgtccgattgtaacaagatatgttgatcctacgtaaggatattaaagtatgtattcatcactaatataatcagtgtattccaatatgtactacgatttccaatgtctttattgtcgccgtatgtaatcggcgtcacaaaataatccccggtgactttcttttaatccaggatgaaataatatgttattataatttttgcgatttggtccgttataggaattgaagtgtgcttgaggtcggtcgccaccactcccatttcataattttacatgtatttgaaaaataaaaatttatggtattcaatttaaacacgtatacttgtaaagaatgatatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatggtgtaatacataggaattcgtttaaacgatctgcgtctaattttcggtccaacttgcacaggaaagacgtcgaccgcggtagctcttgcccagcagactgggcttccagtcctttcgctcgatcgggtccaatgttgtcctcagctgtgaaccggaagcggacgaccaacagtggaagaactgaaaggaacgagccgtctataccttgatgatcggcctctggtgaagggtatcatcgcagccaagcaagctcatgaaaggctgatgggggaggtgtataattatgaggcccacggcgggcttattctttagggaggatctatctcgttgctcaagtgcatggcgcaaagcagttattggagtgcggattttcgttggcatattattcgccacgagttagcagacgaagagaccttcatgaacgtggccaaggccagagttaagcagatgttacgccctgctgcaggcctttctattatccaatagttggttgatctttggaaagagcctcggctgaggcccatactgaaagagatcgatggatatcgatatgccatgttgtttgctagccagaaccagatcacatccgatatgctattgcagcttgacgcagatatggaggataagttgattcatgggatcgctcaggagtagctcatccatgcacgccgacaagaacagaaattccgtcgagttaacgcagccgcttacgacggattcgaaggtcatccattcggaatgtattagtttgcaccagctccgcgtcacacctgtcttcatttgaataagatgttagcaattgtttttagctttgtcttgttgtggcagggcggcaagtgcttcagacatcattctgttttcaaattttatgctggagaacagcttcttaattcctttggaaataatagactgcgtcttaaaattcagatgtctggatatagatatgattgtaaaataacctatttaagtgtcatttagaacataagttttatgaatgttcttccattttcgtcatcgaacgaataagagtaaatacaccttttttaacattacaaataagttcttatacgttgtttatacaccgggaatcatttccattattttcgcgcaaaagtcacggatattcgtgaaagcgacataaactgcgaaatttgcggggagtgtcttgagtttgcctcgaggctagcgcatgcacatagacacacacatcatctcattgatgcttggtaataattgtcattagattgtttttatgcatagatgcactcgaaatcagccaattttagacaagtatcaaacggatgtgacttcagtacattaaaaacgtccgcaatgtgttattaagttgtctaagcgtcaatttgatttacaattgaatatatcctgccccagccagccaacagctcgatttacaattgaatatatcctgccggccggcccacgcgtgtcgaggaattctgatctggcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttaaacatcatccgtcggatggcgttgcgagagaagcagtcgatccgtgagatcagccgacgcaccgggcaggcgcgcaacacgatcgcaaagtatttgaacgcaggtacaatcgagccgacgttcacgcggaacgaccaagcaagcttggctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaagggtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagcccccgagcgcttagtgggaatttgtaccccttatcgaaccgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtcttgcgatggcatttcgatccattcccattccgcgctcaagatggcttcccctcggcagttcatcagggctaaatcaatctagccgacttgtccggtgaaatgggctgcactccaacagaaacaatcaaacaaacatacacagcgacttattcacacgagctcaaattacaacggtatatatcctgccagtcagcatcatcacaccaaaagttaggcccgaatagtttgaaattagaaagctcgcaattgaggtctacaggccaaattcgctcttagccgtacaatattactcaccggatcctaaccggtgtgatcatgggccgcgattaaaaatctcaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaacctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaatgaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggccaccgcttaattaaggcgcgccatgcccgggcaagcggccgcacaagtttgtacaaaaaagcaggctccgcggccgcactaggtttaaactctagaagctaggaattcgcggccgctttcctgcatgacatcgtcctgcagagccaagcgcatgcttaattaaactagtctcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataatgatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatgatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataagatttatggtggactaattttcatatatttcttattgcttttaccttttcttggtatgtaagtccgtaactggaattactgtgggttgccatgacactctgtggtcttttggttcatgcatggatcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctaggtaccctgcagggatccaacaatggctcccaacatttctgaggatgtcaatggtgttctttttgagtcagatgcggcaacccctgatttggctctttccacaccacctgtgcaaaaagctgacaacagacccaagcaacttgtgtggaggaacattttgcttttcgcttacttgcacctcgcagctctctacggaggctatttgtttctcttcagtgcaaaatggcagaccgacattttcgcttacattctttatgtcatctctggactggggataactgctggggcacatagactctgggctcacaagtcatacaaagccaagtggccactcagagttatactggtcatcttcaacacggttgcctttcaagacgctgctatggattgggctcgtgaccatagaatgcatcacaagtacagcgagaccgacgcggacccacacaatgcaacgagaggtttcttcttctctcacattggctggcttcttgttaggaaacatcctgatctgaaagaaaaagggaagggactcgacatgagtgatctccttgctgatccaatactccgttttcagaagaagtactatctgatcctcatgcctctggcctgttttgtgatgccaaccgttatcccggtttacttttggggagaaacttggacaaatgctttcttcgtggcagccatgttccgttatgctttcatcctgaatgttacctggttggtgaactctgccgcacacaagtggggagacaaaccctatgacaagtccatcaagccttccgaaaacctttcagttgcgatgtttgctttgggagaaggatttcacaattaccatcacacttttccgtgggactacaagacagcagagcttggaaacaacaagttgaacttcacaacaacgttcatcaatttctttgcgaaaatcggttgggcctatgatttgaagactgtgagtgatgacattgtcaagaacagggtcaagagaactggcgatggaagccatcatctctggggctggggtgatgagaatcagagcaaagaagagatagatgcagccattaggatcaaccctaaagacgattgagtagttagcttaatcacctagagctcggtcacctcgagtatcaaaatctatttagaaatacacaatattttgttgcaggcttgctggagaatcgatctgctatcataaaaattacaaaaaaattttatttgcctcaattattttaggattggtattaaggacgcttaaattatttgtcgggtcactacgcatcattgtgattgagaagatcagcgatacgaaatattcgtagtactatcgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaaggttggcgcgccgacccagctttc

SEQ ID NO: 64 - DNA, 14575 bp, Artificial
  Plasmid pDAB7328
ttgtacaaagtggttgcggccgcttaattaaatttaaattcaattaatgcaatcttgattttcaacaacgaaggtaatggcgtaaaagaaaaaatgtatgttattgtattgatctttcatgatgttgaagcgtgccataatatgatgatgtataattaaaatattaactgtcgcattttattgaaatggcactgttatttcaaccatatctttgattctgttacatgacacgactgcaagaagtaaataatagacgccgttgttaaagaattgctatcatatgtgcctaactagagggaatttgagcgtcagacctaatcaaatattacaaaatatctcactctgtcgccagcaatggtgtaatcagcgcagacaaatggcgtaaagatcgcggaaaaacctccccgagtggcatgatagctgcctctgtattgctgatttagtcagccttatttgacttaagggtgccctcgttagtgacaaattgctttcaaggagacagccatgccccacactttgttgaaaaacaaattgcctttggggagacggtaaagccagttgctcttcaataaggaatgtcgaggaggcaatgtaaccgcctctggtagtacacttctctaatccaaaaatcaatttgtattcaagataccgcaaaaaacttatggtttaaaccctgcaggactagtccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtaggtaccagatctggatcccaaaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatctacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccaaatctgagtagttagcttaatcacctagagctcgatcggcggcaatagcttcttagcgccatcccgggttgatcctatctgtgttgaaatagttgcggtgggcaaggctctctttcagaaagacaggcggccaaaggaacccaaggtgaggtgggctatggctctcagttccttgtggaagcgcttggtctaaggtgcagaggtgttagcgggatgaagcaaaagtgtccgattgtaacaagatatgttgatcctacgtaaggatattaaagtatgtattcatcactaatataatcagtgtattccaatatgtactacgatttccaatgtctttattgtcgccgtatgtaatcggcgtcacaaaataatccccggtgactttcttttaatccaggatgaaataatatgttattataatttttgcgatttggtccgttataggaattgaagtgtgcttgaggtcggtcgccaccactcccatttcataattttacatgtatttgaaaaataaaaatttatggtattcaatttaaacacgtatacttgtaaagaatgatatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatggtgtaatacataggaattcgtttaaacgatctgcgtctaattttcggtccaacttgcacaggaaagacgtcgaccgcggtagctcttgcccagcagactgggcttccagtcctttcgctcgatcgggtccaatgttgtcctcagctgtgaaccggaagcggacgaccaacagtggaagaactgaaaggaacgagccgtctataccttgatgatcggcctctggtgaagggtatcatcgcagccaagcaagctcatgaaaggctgatgggggaggtgtataattatgaggcccacggcgggcttattctttagggaggatctatctcgttgctcaagtgcatggcgcaaagcagttattggagtgcggattttcgttggcatattattcgccacgagttagcagacgaagagaccttcatgaacgtggccaaggccagagttaagcagatgttacgccctgctgcaggcctttctattatccaatagttggttgatctttggaaagagcctcggctgaggcccatactgaaagagatcgatggatatcgatatgccatgttgtttgctagccagaaccagatcacatccgatatgctattgcagcttgacgcagatatggaggataagttgattcatgggatcgctcaggagtagctcatccatgcacgccgacaagaacagaaattccgtcgagttaacgcagccgcttacgacggattcgaaggtcatccattcggaatgtattagtttgcaccagctccgcgtcacacctgtcttcatttgaataagatgttagcaattgtttttagctttgtcttgttgtggcagggcggcaagtgcttcagacatcattctgttttcaaattttatgctggagaacagcttcttaattcctttggaaataatagactgcgtcttaaaattcagatgtctggatatagatatgattgtaaaataacctatttaagtgtcatttagaacataagttttatgaatgttcttccattttcgtcatcgaacgaataagagtaaatacaccttttttaacattacaaataagttcttatacgttgtttatacaccgggaatcatttccattattttcgcgcaaaagtcacggatattcgtgaaagcgacataaactgcgaaatttgcggggagtgtcttgagtttgcctcgaggctagcgcatgcacatagacacacacatcatctcattgatgcttggtaataattgtcattagattgtttttatgcatagatgcactcgaaatcagccaattttagacaagtatcaaacggatgtgacttcagtacattaaaaacgtccgcaatgtgttattaagttgtctaagcgtcaatttgatttacaattgaatatatcctgccccagccagccaacagctcgatttacaattgaatatatcctgccggccggcccacgcgtgtcgaggaattctgatctggcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttaaacatcatccgtcggatggcgttgcgagagaagcagtcgatccgtgagatcagccgacgcaccgggcaggcgcgcaacacgatcgcaaagtatttgaacgcaggtacaatcgagccgacgttcacgcggaacgaccaagcaagcttggctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaagggtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagcccccgagcgcttagtgggaatttgtaccccttatcgaaccgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtcttgcgatggcatttcgatccattcccattccgcgctcaagatggcttcccctcggcagttcatcagggctaaatcaatctagccgacttgtccggtgaaatgggctgcactccaacagaaacaatcaaacaaacatacacagcgacttattcacacgagctcaaattacaacggtatatatcctgccagtcagcatcatcacaccaaaagttaggcccgaatagtttgaaattagaaagctcgcaattgaggtctacaggccaaattcgctcttagccgtacaatattactcaccggatcctaaccggtgtgatcatgggccgcgattaaaaatctcaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaacctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaatgaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggccaccgcttaattaaggcgcgccatgcccgggcaagcggccgcacaagtttgtacaaaaaagcaggctccgcggccgcactaggtttaaactctagaagctaggaattcgcggccgctttcctgcatgacatcgtcctgcagagccaagcgcatgcttaattaaactagtctcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataatgatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatgatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataagatttatggtggactaattttcatatatttcttattgcttttaccttttcttggtatgtaagtccgtaactggaattactgtgggttgccatgacactctgtggtcttttggttcatgcatggatcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctaggtaccctgcagggatccaacaatgtctgctccaaccgctgacatcagggctagggctccagaggctaagaaggttcacatcgctgataccgctatcaacaggcacaattggtacaagcacgtgaactggctcaacgtcttcctcatcatcggaatcccactctacggatgcatccaagctttctgggttccacttcaactcaagaccgctatctgggctgtgatctactacttcttcaccggacttggaatcaccgctggataccacaggctttgggctcactgctcatactctgctactcttccacttaggatctggcttgctgctgttggaggaggagctgttgagggatctatcagatggtgggctagggatcacagggctcatcataggtacaccgataccgacaaggacccatactctgttaggaagggacttctctactctcaccttggatggatggtgatgaagcagaacccaaagaggatcggaaggaccgacatctctgatctcaacgaggacccagttgttgtttggcaacacaggaactacctcaaggttgtgttcaccatgggacttgctgttccaatgcttgttgctggacttggatggggagattggcttggaggattcgtgtacgctggaatccttaggatcttcttcgttcaacaagctaccttctgcgtgaactctcttgctcactggcttggagatcaaccattcgatgataggaactctcctagggatcacgtgatcaccgctcttgttacccttggagagggataccacaacttccaccacgagttcccatctgactacaggaacgctatcgagtggcaccagtacgatcctaccaagtggtctatctgggcttggaagcaacttggattggcttacgatctcaagaagttcagggctaacgagatcgagaagggaagggttcaacaacttcagaagaagcttgataggaagagggctactcttgattggggaaccccacttgatcaacttccagtgatggaatgggatgactacgttgagcaagctaagaacggaaggggacttgttgctatcgctggagttgttcacgatgttaccgacttcatcaaggatcacccaggaggaaaggctatgatctcttctggaatcggaaaggatgctaccgctatgttcaacggaggagtgtactaccactctaacgcagctcacaaccttcttagcaccatgagggtgggagtgatcaggggaggatgcgaggttgagatctggaagagggctcagaaggagaacgttgagtacgttagggatggatctggacaaagggtgatcagggctggagagcaaccaaccaagatcccagagccaatcccaaccgctgatgctgcttgagtagttagcttaatcacctaggtcaccagtatgaactaaaatgcatgtaggtgtaagagctcggtcacctcgagtatcaaaatctatttagaaatacacaatattttgttgcaggcttgctggagaatcgatctgctatcataaaaattacaaaaaaattttatttgcctcaattattttaggattggtattaaggacgcttaaattatttgtcgggtcactacgcatcattgtgattgagaagatcagcgatacgaaatattcgtagtactatcgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaaggttggcgcgccgacccagctttc

SEQ ID NO: 65 - DNA, 14602 bp, Artificial
  Plasmid pDAB7330
ttgtacaaagtggttgcggccgcttaattaaatttaaattcaattaatgcaatcttgattttcaacaacgaaggtaatggcgtaaaagaaaaaatgtatgttattgtattgatctttcatgatgttgaagcgtgccataatatgatgatgtataattaaaatattaactgtcgcattttattgaaatggcactgttatttcaaccatatctttgattctgttacatgacacgactgcaagaagtaaataatagacgccgttgttaaagaattgctatcatatgtgcctaactagagggaatttgagcgtcagacctaatcaaatattacaaaatatctcactctgtcgccagcaatggtgtaatcagcgcagacaaatggcgtaaagatcgcggaaaaacctccccgagtggcatgatagctgcctctgtattgctgatttagtcagccttatttgacttaagggtgccctcgttagtgacaaattgctttcaaggagacagccatgccccacactttgttgaaaaacaaattgcctttggggagacggtaaagccagttgctcttcaataaggaatgtcgaggaggcaatgtaaccgcctctggtagtacacttctctaatccaaaaatcaatttgtattcaagataccgcaaaaaacttatggtttaaaccctgcaggactagtccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtaggtaccagatctggatcccaaaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatctacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccaaatctgagtagttagcttaatcacctagagctcgatcggcggcaatagcttcttagcgccatcccgggttgatcctatctgtgttgaaatagttgcggtgggcaaggctctctttcagaaagacaggcggccaaaggaacccaaggtgaggtgggctatggctctcagttccttgtggaagcgcttggtctaaggtgcagaggtgttagcgggatgaagcaaaagtgtccgattgtaacaagatatgttgatcctacgtaaggatattaaagtatgtattcatcactaatataatcagtgtattccaatatgtactacgatttccaatgtctttattgtcgccgtatgtaatcggcgtcacaaaataatccccggtgactttcttttaatccaggatgaaataatatgttattataatttttgcgatttggtccgttataggaattgaagtgtgcttgaggtcggtcgccaccactcccatttcataattttacatgtatttgaaaaataaaaatttatggtattcaatttaaacacgtatacttgtaaagaatgatatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatggtgtaatacataggaattcgtttaaacgatctgcgtctaattttcggtccaacttgcacaggaaagacgtcgaccgcggtagctcttgcccagcagactgggcttccagtcctttcgctcgatcgggtccaatgttgtcctcagctgtgaaccggaagcggacgaccaacagtggaagaactgaaaggaacgagccgtctataccttgatgatcggcctctggtgaagggtatcatcgcagccaagcaagctcatgaaaggctgatgggggaggtgtataattatgaggcccacggcgggcttattctttagggaggatctatctcgttgctcaagtgcatggcgcaaagcagttattggagtgcggattttcgttggcatattattcgccacgagttagcagacgaagagaccttcatgaacgtggccaaggccagagttaagcagatgttacgccctgctgcaggcctttctattatccaatagttggttgatctttggaaagagcctcggctgaggcccatactgaaagagatcgatggatatcgatatgccatgttgtttgctagccagaaccagatcacatccgatatgctattgcagcttgacgcagatatggaggataagttgattcatgggatcgctcaggagtagctcatccatgcacgccgacaagaacagaaattccgtcgagttaacgcagccgcttacgacggattcgaaggtcatccattcggaatgtattagtttgcaccagctccgcgtcacacctgtcttcatttgaataagatgttagcaattgtttttagctttgtcttgttgtggcagggcggcaagtgcttcagacatcattctgttttcaaattttatgctggagaacagcttcttaattcctttggaaataatagactgcgtcttaaaattcagatgtctggatatagatatgattgtaaaataacctatttaagtgtcatttagaacataagttttatgaatgttcttccattttcgtcatcgaacgaataagagtaaatacaccttttttaacattacaaataagttcttatacgttgtttatacaccgggaatcatttccattattttcgcgcaaaagtcacggatattcgtgaaagcgacataaactgcgaaatttgcggggagtgtcttgagtttgcctcgaggctagcgcatgcacatagacacacacatcatctcattgatgcttggtaataattgtcattagattgtttttatgcatagatgcactcgaaatcagccaattttagacaagtatcaaacggatgtgacttcagtacattaaaaacgtccgcaatgtgttattaagttgtctaagcgtcaatttgatttacaattgaatatatcctgccccagccagccaacagctcgatttacaattgaatatatcctgccggccggcccacgcgtgtcgaggaattctgatctggcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttaaacatcatccgtcggatggcgttgcgagagaagcagtcgatccgtgagatcagccgacgcaccgggcaggcgcgcaacacgatcgcaaagtatttgaacgcaggtacaatcgagccgacgttcacgcggaacgaccaagcaagcttggctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaagggtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagcccccgagcgcttagtgggaatttgtaccccttatcgaaccgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtcttgcgatggcatttcgatccattcccattccgcgctcaagatggcttcccctcggcagttcatcagggctaaatcaatctagccgacttgtccggtgaaatgggctgcactccaacagaaacaatcaaacaaacatacacagcgacttattcacacgagctcaaattacaacggtatatatcctgccagtcagcatcatcacaccaaaagttaggcccgaatagtttgaaattagaaagctcgcaattgaggtctacaggccaaattcgctcttagccgtacaatattactcaccggatcctaaccggtgtgatcatgggccgcgattaaaaatctcaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaacctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaatgaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggccaccgcttaattaaggcgcgccatgcccgggcaagcggccgcacaagtttgtacaaaaaagcaggctccgcggccgcactaggtttaaactctagaagctaggaattcgcggccgctttcctgcatgacatcgtcctgcagagccaagcgcatgcttaattaaactagtctcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataatgatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatgatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataagatttatggtggactaattttcatatatttcttattgcttttaccttttcttggtatgtaagtccgtaactggaattactgtgggttgccatgacactctgtggtcttttggttcatgcatggatcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctaggtaccctgcagggatccaacaatggccagcagttcttcaagtgtgccagaacttgccgcagctttccctgatgggacaacggacttcaaacccatgaggaacaccaaaggctatgatgtctccaaacctcacatctctgaaacaccgatgactttgaagaactggcacaaacatgtgaactggctcaacaccacattcattctctttgttccactggctgggttgatctcaacctattgggttcctcttcaatggaaaactgcagtgtgggcagttgtgtactacttcaacactggacttgggatcactgctggctaccatagattgtgggcacattcctcttacaaggccagcttgcctctcaaaatctaccttgccgcagttggtgctggagccgttgaaggttccataagatggtggagcaacggacacagagcacatcacagatacacagacacagagaaagatccttactcagtgaggaagggattgctctacagccacatgggttggatgctcttgaagcagaatccaaagaagcaagggaggacggacattactgatctgaatgaggacccagttgtggtctggcaacataggaactttctcaagtgtgtgatcttcatggctttggtctttcccacccttgttgctggcctgggatggggagactactggggaggtttcatctatggagggatcttgagagtgttctttgttcagcaagccaccttctgtgtcaactcacttgcacattggcttggtgatcaaccgtttgatgacagaaactctccacgtgaccatgtcataactgctcttgtcacgctgggtgaaggctatcacaactttcaccatgagtttccgtcagactatagaaatgcgattgagtggtatcagtatgaccccacgaagtggagcatttggatttggaagcaacttggacttgctcacaatctcaagcagttcagacagaatgagatagagaagggaagggttcaacagttgcagaagaaactggatcagaagagagcgaaacttgattggggaataccgttggaacaactccctgttgtgtcttgggatgactttgttgaacagtcaaagaatggcaaggcatggattgctgttgctggtgtcattcacgatgttggtgacttcatcaaggatcatcctggtggacgtgctctcatcaactctgcgattggcaaagatgccacagcgatcttcaatggaggtgtctacaatcattcaaatgccgcacacaaccttctctccaccatgagggttggtgtcctccgtggagggtgcgaagtggagatatggaaacgtgctcaaagtgagaacaaagatgtctctactgtggttgatagttctggcaaccgtattgtgagagctggtggacaagctaccaaagtggttcagccagtccctggtgctcaagcagcttgatgagtagttagcttaatcacctagagctcggtcacctcgagtatcaaaatctatttagaaatacacaatattttgttgcaggcttgctggagaatcgatctgctatcataaaaattacaaaaaaattttatttgcctcaattattttaggattggtattaaggacgcttaaattatttgtcgggtcactacgcatcattgtgattgagaagatcagcgatacgaaatattcgtagtactatcgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaaggttggcgcgccgacccagctttc

SEQ ID NO: 66 - DNA, 10915 bp, Artificial
  Plasmid pDAB7331
taaatttaaattcaattaatgcaatcttgattttcaacaacgaaggtaatggcgtaaaagaaaaaatgtatgttattgtattgatctttcatgatgttgaagcgtgccataatatgatgatgtataattaaaatattaactgtcgcattttattgaaatggcactgttatttcaaccatatctttgattctgttacatgacacgactgcaagaagtaaataatagacgccgttgttaaagaattgctatcatatgtgcctaactagagggaatttgagcgtcagacctaatcaaatattacaaaatatctcactctgtcgccagcaatggtgtaatcagcgcagacaaatggcgtaaagatcgcggaaaaacctccccgagtggcatgatagctgcctctgtattgctgatttagtcagccttatttgacttaagggtgccctcgttagtgacaaattgctttcaaggagacagccatgccccacactttgttgaaaaacaaattgcctttggggagacggtaaagccagttgctcttcaataaggaatgtcgaggaggcaatgtaaccgcctctggtagtacacttctctaatccaaaaatcaatttgtattcaagataccgcaaaaaacttatggtttaaaccctgcaggactagtccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtaggtaccagatctggatcccaaaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatctacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccaaatctgagtagttagcttaatcacctagagctcgatcggcggcaatagcttcttagcgccatcccgggttgatcctatctgtgttgaaatagttgcggtgggcaaggctctctttcagaaagacaggcggccaaaggaacccaaggtgaggtgggctatggctctcagttccttgtggaagcgcttggtctaaggtgcagaggtgttagcgggatgaagcaaaagtgtccgattgtaacaagatatgttgatcctacgtaaggatattaaagtatgtattcatcactaatataatcagtgtattccaatatgtactacgatttccaatgtctttattgtcgccgtatgtaatcggcgtcacaaaataatccccggtgactttcttttaatccaggatgaaataatatgttattataatttttgcgatttggtccgttataggaattgaagtgtgcttgaggtcggtcgccaccactcccatttcataattttacatgtatttgaaaaataaaaatttatggtattcaatttaaacacgtatacttgtaaagaatgatatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatggtgtaatacataggaattcgtttaaacgatctgcgtctaattttcggtccaacttgcacaggaaagacgtcgaccgcggtagctcttgcccagcagactgggcttccagtcctttcgctcgatcgggtccaatgttgtcctcagctgtgaaccggaagcggacgaccaacagtggaagaactgaaaggaacgagccgtctataccttgatgatcggcctctggtgaagggtatcatcgcagccaagcaagctcatgaaaggctgatgggggaggtgtataattatgaggcccacggcgggcttattctttagggaggatctatctcgttgctcaagtgcatggcgcaaagcagttattggagtgcggattttcgttggcatattattcgccacgagttagcagacgaagagaccttcatgaacgtggccaaggccagagttaagcagatgttacgccctgctgcaggcctttctattatccaatagttggttgatctttggaaagagcctcggctgaggcccatactgaaagagatcgatggatatcgatatgccatgttgtttgctagccagaaccagatcacatccgatatgctattgcagcttgacgcagatatggaggataagttgattcatgggatcgctcaggagtagctcatccatgcacgccgacaagaacagaaattccgtcgagttaacgcagccgcttacgacggattcgaaggtcatccattcggaatgtattagtttgcaccagctccgcgtcacacctgtcttcatttgaataagatgttagcaattgtttttagctttgtcttgttgtggcagggcggcaagtgcttcagacatcattctgttttcaaattttatgctggagaacagcttcttaattcctttggaaataatagactgcgtcttaaaattcagatgtctggatatagatatgattgtaaaataacctatttaagtgtcatttagaacataagttttatgaatgttcttccattttcgtcatcgaacgaataagagtaaatacaccttttttaacattacaaataagttcttatacgttgtttatacaccgggaatcatttccattattttcgcgcaaaagtcacggatattcgtgaaagcgacataaactgcgaaatttgcggggagtgtcttgagtttgcctcgaggctagcgcatgcacatagacacacacatcatctcattgatgcttggtaataattgtcattagattgtttttatgcatagatgcactcgaaatcagccaattttagacaagtatcaaacggatgtgacttcagtacattaaaaacgtccgcaatgtgttattaagttgtctaagcgtcaatttgatttacaattgaatatatcctgccccagccagccaacagctcgatttacaattgaatatatcctgccggccggcccacgcgtgtcgaggaattctgatctggcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttaaacatcatccgtcggatggcgttgcgagagaagcagtcgatccgtgagatcagccgacgcaccgggcaggcgcgcaacacgatcgcaaagtatttgaacgcaggtacaatcgagccgacgttcacgcggaacgaccaagcaagcttggctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaagggtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagcccccgagcgcttagtgggaatttgtaccccttatcgaaccgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtcttgcgatggcatttcgatccattcccattccgcgctcaagatggcttcccctcggcagttcatcagggctaaatcaatctagccgacttgtccggtgaaatgggctgcactccaacagaaacaatcaaacaaacatacacagcgacttattcacacgagctcaaattacaacggtatatatcctgccagtcagcatcatcacaccaaaagttaggcccgaatagtttgaaattagaaagctcgcaattgaggtctacaggccaaattcgctcttagccgtacaatattactcaccggatcctaaccggtgtgatcatgggccgcgattaaaaatctcaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaacctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaatgaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggccaccgcttaat

SEQ ID NO: 67 - DNA, 1448 bp, Artificial
  PvPhas v2 promoter
ctcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataatgatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatgatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataagatttatggtggactaattttcatatatttcttattgcttttaccttttcttggtatgtaagtccgtaactggaattactgtgggttgccatgacactctgtggtcttttggttcatgcatggatcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcact

SEQ ID NO: 68 - DNA, 85 bp, Artificial
  PvPhas 5′ UTR
cacttctttcatcatccatccatccagagtactactactctactactataataccccaacccaactcatattcaatactactcta

SEQ ID NO: 69 - DNA, 129 bp, Artificial
  PvPhas 3′ UTR v1
agtatgaactaaaatgcatgtaggtgtaagagctcatggagagcatggaatattgtatccgaccatgtaacagtataataactgagctccatctcacttcttctatgaataaacaaaggatgttatgat

SEQ ID NO: 70 - DNA, 1088 bp, Artificial
  PvPhas 3′ MAR v2
atattaacactctatctatgcaccttattgttctatgataaatttcctcttattattataaatcatctgaatcgtgacggcttatggaatgcttcaaatagtacaaaaacaaatgtgtactataagactttctaaacaattctaactttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaattttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacccttatacaaaaccccaaaaacaagtttcctagcaccctaccaacgaattc

SEQ ID NO: 71 - DNA, 457 bp, Artificial
  AtuORF 3′ UTR v1
tatcaaaatctatttagaaatacacaatattttgttgcaggcttgctggagaatcgatctgctatcataaaaattacaaaaaaattttatttgcctcaattattttaggattggtattaaggacgcttaaattatttgtcgggtcactacgcatcattgtgattgagaagatcagcgatacgaaatattcgtagtactatcgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaaggtt

SEQ ID NO: 72 - protein, 68 aa, Artificial
  N-terminal residues 1-68 of AnD9DS
MSAPTADIRARAPEAKKVHIADTAINRHNWYKHVNWLNVFLIIGIPLYGCIQAFWVPLQLKTAIWAVI

SEQ ID NO: 73 - protein, 175 aa, Artificial
  C-terminal residues 281-455 of AnD9DS
SIWAWKQLGLAYDLKKFRANEIEKGRVQQLQKKLDRKRATLDWGTPLDQLPVMEWDDYVEQAKNGRGLVAIAGVVHDVTDFIKDHPGGKAMISSGIGKDATAMFNGGVYYHSNAAHNLLSTMRVGVIRGGCEVEIWKRAQKENVEYVRDGSGQRVIRAGEQPTKIPEPIPTADAA

SEQ ID NO: 74 - DNA, 14103 bp, Artificial
  Plasmid pDAB110110
gtacaaaaaagcaggcttctagacctaggtggagtcatcacgcagactatctcagcatgtgcgtagcacgcggccgcctcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataatgatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatgatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataagatttatggtggactaattttcatatatttcttattgcttttaccttttcttggtatgtaagtccgtaactggaattactgtgggttgccatgacactctgtggtcttttggttcatgcatggatcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctaggatccaacaatggctgcacttgatagcatccctgaggacaaagcaactagctccaagtcaacccacatacagtaccaagaggtcacgtttaggaactggtacaagaaaatcaactggctcaacacgacccttgttgtcctcattcctgctcttgggttgtacttgacgagaaccacacctctcaccagacctaccctcatttggtctgttctctactatttctgtacagcgtttggcatcactggtggctaccacagactttggtcccataggtcttacagtgcgaggttgccattgagactcttcctggctttcactggagctggtgcgatccaaggttctgcaagatggtggtcagccaatcatagggcacatcaccgttggacggacaccatgaaggacccctactctgtgatgagaggactgctgttctcccacataggttggatggttctcaactctgatccaaaggtcaaaggcagaacagatgtttctgatcttgactctgatcccgtcgttgtgtggcaacacaaacactatggcaagtgtttgctctttgccgcttggatctttccgatgatagtggctgggctgggttggggagattggtggggtggacttgtctatgctggcatcatacgtgcctgctttgttcagcaagccactttctgtgtcaactcattggcacattggataggtgaacaaccgtttgatgacagacgtactccaagggatcatgttctgactgcgttggtcacaatgggagaaggataccacaacttccaccatgagtttccgagtgactacagaaatgccatcatttggtatcagtatgaccctacaaagtggctcatctatctcttcagcttgggtcccttcccattggcctactctctcaagaccttccgttccaatgagattgagaaaggaaggcttcagcaacagcaaaaggctcttgacaagaaaagaagtggtcttgattggggacttcctctcttccagcttccagtgatctcatgggatgactttcaagctcgttgcaaagaaagtggagagatgcttgttgctgttgctggagtgatccatgatgtctcccagttcattgaagatcatcctggtgggaggagcctcattagaagtgctgttgggaaagatgggactggcatgttcaatggtggagtgtatgaacattcaaacgccgcacacaacttgctgagcacaatgagagttggagtcttgagaggtggacaagaagtggaggtttggaagaaacagagggtggatgttcttgggaagtcagacattcttcgtcaagtgacaagggtggagcgtctggtggaaggagctgttgcagcgtgatgagtagttagcttaatcacctagagctcggtcaccagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaactttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaattttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacccttatacaaaaccccaaaaacaagtttcctagcaccctaccaacgaattcgcggccgcttaattaagatgagtgatactcaggactcaggactcactctgctgatcactagtgctagcctcgaggtcgaccagctttcttgtacaaagtggttgcggccgcttaattaaatttaaattcaattaatgcaatcttgattttcaacaacgaaggtaatggcgtaaaagaaaaaatgtatgttattgtattgatctttcatgatgttgaagcgtgccataatatgatgatgtataattaaaatattaactgtcgcattttattgaaatggcactgttatttcaaccatatctttgattctgttacatgacacgactgcaagaagtaaataatagacgccgttgttaaagaattgctatcatatgtgcctaactagagggaatttgagcgtcagacctaatcaaatattacaaaatatctcactctgtcgccagcaatggtgtaatcagcgcagacaaatggcgtaaagatcgcggaaaaacctccccgagtggcatgatagctgcctctgtattgctgatttagtcagccttatttgacttaagggtgccctcgttagtgacaaattgctttcaaggagacagccatgccccacactttgttgaaaaacaaattgcctttggggagacggtaaagccagttgctcttcaataaggaatgtcgaggaggcaatgtaaccgcctctggtagtacacttctctaatccaaaaatcaatttgtattcaagataccgcaaaaaacttatggtttaaaccctgcaggactagtccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtaggtaccagatctggatcccaaaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatctacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccaaatctgagtagttagcttaatcacctagagctcgatcggcggcaatagcttcttagcgccatcccgggttgatcctatctgtgttgaaatagttgcggtgggcaaggctctctttcagaaagacaggcggccaaaggaacccaaggtgaggtgggctatggctctcagttccttgtggaagcgcttggtctaaggtgcagaggtgttagcgggatgaagcaaaagtgtccgattgtaacaagatatgttgatcctacgtaaggatattaaagtatgtattcatcactaatataatcagtgtattccaatatgtactacgatttccaatgtctttattgtcgccgtatgtaatcggcgtcacaaaataatccccggtgactttcttttaatccaggatgaaataatatgttattataatttttgcgatttggtccgttataggaattgaagtgtgcttgaggtcggtcgccaccactcccatttcataattttacatgtatttgaaaaataaaaatttatggtattcaatttaaacacgtatacttgtaaagaatgatatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatggtgtaatacataggaattcgtttaaacgatctgcgtctaattttcggtccaacttgcacaggaaagacgtcgaccgcggtagctcttgcccagcagactgggcttccagtcctttcgctcgatcgggtccaatgttgtcctcagctgtgaaccggaagcggacgaccaacagtggaagaactgaaaggaacgagccgtctataccttgatgatcggcctctggtgaagggtatcatcgcagccaagcaagctcatgaaaggctgatgggggaggtgtataattatgaggcccacggcgggcttattctttagggaggatctatctcgttgctcaagtgcatggcgcaaagcagttattggagtgcggattttcgttggcatattattcgccacgagttagcagacgaagagaccttcatgaacgtggccaaggccagagttaagcagatgttacgccctgctgcaggcctttctattatccaatagttggttgatctttggaaagagcctcggctgaggcccatactgaaagagatcgatggatatcgatatgccatgttgtttgctagccagaaccagatcacatccgatatgctattgcagcttgacgcagatatggaggataagttgattcatgggatcgctcaggagtagctcatccatgcacgccgacaagaacagaaattccgtcgagttaacgcagccgcttacgacggattcgaaggtcatccattcggaatgtattagtttgcaccagctccgcgtcacacctgtcttcatttgaataagatgttagcaattgtttttagctttgtcttgttgtggcagggcggcaagtgcttcagacatcattctgttttcaaattttatgctggagaacagcttcttaattcctttggaaataatagactgcgtcttaaaattcagatgtctggatatagatatgattgtaaaataacctatttaagtgtcatttagaacataagttttatgaatgttcttccattttcgtcatcgaacgaataagagtaaatacaccttttttaacattacaaataagttcttatacgttgtttatacaccgggaatcatttccattattttcgcgcaaaagtcacggatattcgtgaaagcgacataaactgcgaaatttgcggggagtgtcttgagtttgcctcgaggctagcgcatgcacatagacacacacatcatctcattgatgcttggtaataattgtcattagattgtttttatgcatagatgcactcgaaatcagccaattttagacaagtatcaaacggatgtgacttcagtacattaaaaacgtccgcaatgtgttattaagttgtctaagcgtcaatttgatttacaattgaatatatcctgccccagccagccaacagctcgatttacaattgaatatatcctgccggccggcccacgcgtgtcgaggaattctgatctggcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttaaacatcatccgtcggatggcgttgcgagagaagcagtcgatccgtgagatcagccgacgcaccgggcaggcgcgcaacacgatcgcaaagtatttgaacgcaggtacaatcgagccgacgttcacgcggaacgaccaagcaagcttggctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaagggtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagcccccgagcgcttagtgggaatttgtaccccttatcgaaccgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtcttgcgatggcatttcgatccattcccattccgcgctcaagatggcttcccctcggcagttcatcagggctaaatcaatctagccgacttgtccggtgaaatgggctgcactccaacagaaacaatcaaacaaacatacacagcgacttattcacacgagctcaaattacaacggtatatatcctgccagtcagcatcatcacaccaaaagttaggcccgaatagtttgaaattagaaagctcgcaattgaggtctacaggccaaattcgctcttagccgtacaatattactcaccggatcctaaccggtttaattaaggcgcgccatgcccgggcaagcggccgcacaagttt

SEQ ID NO: 75 - DNA, 13812 bp, Artificial
  Plasmid pDAB110112
cttgtacaaagtggttgcggccgcttaattaaatttaaattcaattaatgcaatcttgattttcaacaacgaaggtaatggcgtaaaagaaaaaatgtatgttattgtattgatctttcatgatgttgaagcgtgccataatatgatgatgtataattaaaatattaactgtcgcattttattgaaatggcactgttatttcaaccatatctttgattctgttacatgacacgactgcaagaagtaaataatagacgccgttgttaaagaattgctatcatatgtgcctaactagagggaatttgagcgtcagacctaatcaaatattacaaaatatctcactctgtcgccagcaatggtgtaatcagcgcagacaaatggcgtaaagatcgcggaaaaacctccccgagtggcatgatagctgcctctgtattgctgatttagtcagccttatttgacttaagggtgccctcgttagtgacaaattgctttcaaggagacagccatgccccacactttgttgaaaaacaaattgcctttggggagacggtaaagccagttgctcttcaataaggaatgtcgaggaggcaatgtaaccgcctctggtagtacacttctctaatccaaaaatcaatttgtattcaagataccgcaaaaaacttatggtttaaaccctgcaggactagtccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtaggtaccagatctggatcccaaaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatctacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccaaatctgagtagttagcttaatcacctagagctcgatcggcggcaatagcttcttagcgccatcccgggttgatcctatctgtgttgaaatagttgcggtgggcaaggctctctttcagaaagacaggcggccaaaggaacccaaggtgaggtgggctatggctctcagttccttgtggaagcgcttggtctaaggtgcagaggtgttagcgggatgaagcaaaagtgtccgattgtaacaagatatgttgatcctacgtaaggatattaaagtatgtattcatcactaatataatcagtgtattccaatatgtactacgatttccaatgtctttattgtcgccgtatgtaatcggcgtcacaaaataatccccggtgactttcttttaatccaggatgaaataatatgttattataatttttgcgatttggtccgttataggaattgaagtgtgcttgaggtcggtcgccaccactcccatttcataattttacatgtatttgaaaaataaaaatttatggtattcaatttaaacacgtatacttgtaaagaatgatatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatggtgtaatacataggaattcgtttaaacgatctgcgtctaattttcggtccaacttgcacaggaaagacgtcgaccgcggtagctcttgcccagcagactgggcttccagtcctttcgctcgatcgggtccaatgttgtcctcagctgtgaaccggaagcggacgaccaacagtggaagaactgaaaggaacgagccgtctataccttgatgatcggcctctggtgaagggtatcatcgcagccaagcaagctcatgaaaggctgatgggggaggtgtataattatgaggcccacggcgggcttattctttagggaggatctatctcgttgctcaagtgcatggcgcaaagcagttattggagtgcggattttcgttggcatattattcgccacgagttagcagacgaagagaccttcatgaacgtggccaaggccagagttaagcagatgttacgccctgctgcaggcctttctattatccaatagttggttgatctttggaaagagcctcggctgaggcccatactgaaagagatcgatggatatcgatatgccatgttgtttgctagccagaaccagatcacatccgatatgctattgcagcttgacgcagatatggaggataagttgattcatgggatcgctcaggagtagctcatccatgcacgccgacaagaacagaaattccgtcgagttaacgcagccgcttacgacggattcgaaggtcatccattcggaatgtattagtttgcaccagctccgcgtcacacctgtcttcatttgaataagatgttagcaattgtttttagctttgtcttgttgtggcagggcggcaagtgcttcagacatcattctgttttcaaattttatgctggagaacagcttcttaattcctttggaaataatagactgcgtcttaaaattcagatgtctggatatagatatgattgtaaaataacctatttaagtgtcatttagaacataagttttatgaatgttcttccattttcgtcatcgaacgaataagagtaaatacaccttttttaacattacaaataagttcttatacgttgtttatacaccgggaatcatttccattattttcgcgcaaaagtcacggatattcgtgaaagcgacataaactgcgaaatttgcggggagtgtcttgagtttgcctcgaggctagcgcatgcacatagacacacacatcatctcattgatgcttggtaataattgtcattagattgtttttatgcatagatgcactcgaaatcagccaattttagacaagtatcaaacggatgtgacttcagtacattaaaaacgtccgcaatgtgttattaagttgtctaagcgtcaatttgatttacaattgaatatatcctgccccagccagccaacagctcgatttacaattgaatatatcctgccggccggcccacgcgtgtcgaggaattctgatctggcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttaaacatcatccgtcggatggcgttgcgagagaagcagtcgatccgtgagatcagccgacgcaccgggcaggcgcgcaacacgatcgcaaagtatttgaacgcaggtacaatcgagccgacgttcacgcggaacgaccaagcaagcttggctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaagggtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagcccccgagcgcttagtgggaatttgtaccccttatcgaaccgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtcttgcgatggcatttcgatccattcccattccgcgctcaagatggcttcccctcggcagttcatcagggctaaatcaatctagccgacttgtccggtgaaatgggctgcactccaacagaaacaatcaaacaaacatacacagcgacttattcacacgagctcaaattacaacggtatatatcctgccagtcagcatcatcacaccaaaagttaggcccgaatagtttgaaattagaaagctcgcaattgaggtctacaggccaaattcgctcttagccgtacaatattactcaccggatcctaaccggtttaattaaggcgcgccatgcccgggcaagcggccgcacaagtttgtacaaaaaagcaggcttctagacctaggtggagtcatcacgcagactatctcagcatgtgcgtagcacgcggccgcctcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataatgatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatgatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataagatttatggtggactaattttcatatatttcttattgcttttaccttttcttggtatgtaagtccgtaactggaattactgtgggttgccatgacactctgtggtcttttggttcatgcatggatcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctaggatccaacaatggctcccaacatttctgaggatgtcaatggtgttctttttgagtcagatgcggcaacccctgatttggctctttccacaccacctgtgcaaaaagctgacaacagacccaagcaacttgtgtggaggaacattttgcttttcgcttacttgcacctcgcagctctctacggaggctatttgtttctcttcagtgcaaaatggcagaccgacattttcgcttacattctttatgtcatctctggactggggataactgctggggcacatagactctgggctcacaagtcatacaaagccaagtggccactcagagttatactggtcatcttcaacacggttgcctttcaagacgctgctatggattgggctcgtgaccatagaatgcatcacaagtacagcgagaccgacgcggacccacacaatgcaacgagaggtttcttcttctctcacattggctggcttcttgttaggaaacatcctgatctgaaagaaaaagggaagggactcgacatgagtgatctccttgctgatccaatactccgttttcagaagaagtactatctgatcctcatgcctctggcctgttttgtgatgccaaccgttatcccggtttacttttggggagaaacttggacaaatgctttcttcgtggcagccatgttccgttatgctttcatcctgaatgttacctggttggtgaactctgccgcacacaagtggggagacaaaccctatgacaagtccatcaagccttccgaaaacctttcagttgcgatgtttgctttgggagaaggatttcacaattaccatcacacttttccgtgggactacaagacagcagagcttggaaacaacaagttgaacttcacaacaacgttcatcaatttctttgcgaaaatcggttgggcctatgatttgaagactgtgagtgatgacattgtcaagaacagggtcaagagaactggcgatggaagccatcatctctggggctggggtgatgagaatcagagcaaagaagagatagatgcagccattaggatcaaccctaaagacgattgagtagttagcttaatcacctagagctcggtcaccagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaactttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaattttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacccttatacaaaaccccaaaaacaagtttcctagcaccctaccaacgaattcgcggccgcttaattaagatgagtgatactcaggactcaggactcactctgctgatcactagtgctagcctcgaggtcgaccagcttt

SEQ ID NO: 76 - DNA, 1434 bp, Magnaporthe grisea
gaattcatggcttcgtcatcttcctccgtgccggagttggctgccgccttccctgatggcactaccgacttcaagcccatgaggaacaccaagggctacgacgtcagcaagccgcacatttccgagacacctatgacactcaagaactggcataagcacgtcaactggctcaacaccaccttcatcttgtttgtgcccctggctggtctcatatccacttactgggtccctctgcagtggaagacggctgtatgggctgtcgtctactacttcaacaccggcctgggaattactgccggttaccaccgactttgggctcacagctcgtacaaggcctcgcttccgctcaaaatctaccttgccgccgttggcgctggtgccgtcgagggctccatcagatggtggtccaacggtcaccgcgcacaccaccgatacaccgataccgagaaggacccctactcagtccgcaagggtctcctgtactcacacatgggatggatgcttctgaagcagaaccccaagaagcagggccgcaccgacatcaccgacctgaacgaggaccccgttgtcgtttggcagcaccgcaacttcctcaagtgtgttatcttcatggccctcgtcttccccacacttgtggctggccttggctggggtgactactggggaggtttcatctacggaggtattctgcgtgtcttcttcgtccagcaggccaccttctgcgtcaactcgcttgcccactggctcggtgaccagcctttcgacgatcgcaactcgccgcgtgatcacgtcatcacagccctggtcacccttggagagggataccacaacttccaccacgagttcccttcggactaccgcaacgctattgagtggtaccagtatgaccccaccaagtggtcaatctggatctggaagcagcttggtcttgcccacaacctgaagcagttccgccaaaacgagattgagaagggacgcgtccagcagctgcagaagaagctcgaccagaagcgcgccaagcttgattggggtattcccttggagcagcttcccgttgttagctgggatgactttgttgagcagtccaagaacggaaaggcttggattgcagttgccggtgtcatccacgatgttggtgacttcatcaaggaccaccctggtggcagagctctcatcaactcggccattggcaaggacgcaaccgcaatcttcaacggcggtgtttacaaccactccaacgccgctcacaacctgctctcgactatgcgtgtgggtgttttgcgtggcggctgcgaggttgagatctggaagcgcgcccagtccgaaaacaaggacgtctcaaccgtcgttgattcttcgggtaaccgcatcgtccgcgcgggtgggcaagcgaccaaggtcgtccagcctgttccgggtgctcaggccgcgtga

SEQ ID NO: 77 - protein, 212 aa, Leptosphaeria nodorum
YYFCTAFGITGGYHRLWSHRSYSARLPLRLFLAFTGAGAIQGSARWWSANHRAHHRWTDTMKDPYSVMRGLLFSHIGWMVLNSDPKVKGRTDVSDLDSDPVVVWQHKHYGKCLLFAAWIFPMIVAGLGWGDWWGGLVYAGIIRACFVQQATFCVNSLAHWIGEQPFDDRRTPRDHVLTALVTMGEGYHNFHHEFPSDYRNAIIWYQYDPTKW

SEQ ID NO: 78 - protein, 216 aa, Helicoverpa zea
LYVISGLGITAGAHRLWAHKSYKAKWPLRVILVIFNTVAFQDAAMDWARDHRMHHKYSETDADPHNATRGFFFSHIGWLLVRKHPDLKEKGKGLDMSDLLADPILRFQKKYYLILMPLACFVMPTVIPVYFWGETWTNAFFVAAMFRYAFILNVTWLVNSAAHKWGDKPYDKSIKPSENLSVAMFALGEGFHNYHHTFPWDYKTAELGNNKLNFTT